(19) United States
(12) Patent Application Publication
(10) Pub. No.: US 2024/0226211 A9
(43) Pub. Date: Jul. 11, 2024

(54) SEAWEED EXTRACTS, ISOLATED COMPOUNDS, AND METHODS OF TREATMENT

(71) Applicant: University of Florida Research Foundation, Incorporated, Gainesville, FL (US)

(72) Inventors: Hendrik Luesch, Gainesville, FL (US); Michelle S. Bousquet, Gainesville, FL (US)

(73) Assignee: University of Florida Research Foundation, Incorporated, Gainesville, FL (US)

(21) Appl. No.: 18/494,658

(22) Filed: Oct. 25, 2023

Related U.S. Application Data
(63) Continuation of application No. 16/770,558, filed on Jun. 5, 2020, now abandoned.
(60) Provisional application No. 62/595,148, filed on Dec. 6, 2017.

(51) Int. Cl. A61K 36/05; A23L 33/105; A61K 8/34; A61K 8/49; A61K 31/055; A61K 31/085; A61K 31/352; A61P 29/00

(52) U.S. Cl. CPC A61K 36/05; A23L 33/105; A61K 8/347; A61K 8/498; A61K 31/055; A61K 31/085; A61K 31/352; A61P 29/00; A61K 2236/33; A61K 2236/53

(57) ABSTRACT

The instant invention relates to seaweed extract compositions, processes for isolation, isolated active agents, and methods of treating disease, disorders and conditions in a subject, including, reactive oxygen species (ROS)-mediated diseases and diseases mediated through the activation of the Nrf2-ARE (antioxidant response element) pathway, including proliferative diseases and disorders, Alzheimer's disease, stroke, and certain diseases and disorders of aging and associated with aging and exposure, by use of the extracts, compounds, and compositions thereof.

### CROSS-REFERENCE TO RELATED APPLICATIONS

This application is a Continuation of U.S. application Ser. No. 16/770,558, filed Jun. 5, 2020, which is a National Phase Application under 35 U.S.C. 371 of PCT International Application No. PCT/US2018/064345, filed Dec. 6, 2018, which claims priority to U.S. Provisional Application No. 62/595,148, filed Dec. 6, 2017, which are incorporated herein by reference in their entirety.

### STATEMENT OF RIGHTS TO INVENTIONS MADE UNDER FEDERALLY SPONSORED RESEARCH

This invention was made with government support under Grant No. CA133681 awarded by the National Institutes of Health. The government has certain rights in the invention.

### BACKGROUND

This invention relates to seaweed extract compositions, enriched active fractions, isolated active agents, and methods of use for the treatment and/or prevention of reactive oxygen species (ROS)-mediated diseases and diseases alleviated or prevented through the activation of the Nrf2-ARE (antioxidant response element) pathway, such as inflammation, cancer, Alzheimer's disease and other neurodegenerative disorders, stroke, chronic kidney disease, type II diabetes, and aging itself.

In aerobes, reactive oxygen species (ROS) is produced during cellular respiration and energy metabolism [Halliwell, B. Biochemistry of oxidative stress. Biochem Soc Trans. 35:1147-50; 2007]. In a healthy cell, the level of ROS is tightly regulated by the antioxidant defense system. However, upon environmental stress or cellular damage, the cell cannot readily detoxify the ROS generated and may thereby suffer from oxidative stress, which is implicated in the pathogenesis of many diseases, such as inflammation, cancer, Alzheimer's disease and other neurodegenerative disorders, stroke, chronic kidney disease, type II diabetes, and aging [Liu, Y.; Kern, J. T.; Walker, J. R.; Johnson, J. A.; Schultz, P. G.; Luesch, H. A genomic screen for activators of the antioxidant response element. Proc Natl Acad Sci USA. 104:5205-10; 2007; Dinkova-Kostova A T, Massiah M A, Bozak R E, Hicks R J, Talalay P. Potency of Michael reaction acceptors as inducers of enzymes that protect against carcinogenesis depends on their reactivity with sulfhydryl groups. Proc Natl Acad Sci USA 2001; 98:3404-3409; Ramos-Gomez M, Kwak M-K, Dolan P M, Itoh K, Yamamoto M, Talalay P et. al. Sensitivity to carcinogenesis is increased and chemoprotective efficacy of enzyme inducers is lost in nrf2 transcription factor-deficient mice. Proc Natl Acad Sci USA 2001; 98:3410-3415; van Muiswinkel F L, Kuiperij H B. The Nrf2-ARE signaling pathway: promising drug target to combat oxidative stress in neurodegenerative disorders. Curr Drug Targets CNS Neurol Disord 2005; 4:267-281; Dinkova-Kostova, AT, Liby K T, Stephenson K K, Holtzclaw W D, Gao X, Suh N et. al. Extremely potent triterpenoid inducers of the phase 2 response: Correlations of protection against oxidant and inflammatory stress. Proc Natl Acad Sci USA 2005; 102:4584-4589; Chen X-L, Kunsch C. Induction of cytoprotective genes through Nrf2/antioxidant response element pathway: a new therapeutic approach for the treatment of inflammatory diseases. Curr Pharm Des 2004; 10:879-891; Pergola P E, Raskin P, Toto R D, Meyer C J, Huff J W, Grossman E B et. al. BEAM Study Investigators. Bardoxolone methyl and kidney function in CKD with type 2 diabetes. N Engl J Med 2011; 365:327-336].

The foundation of cancer relies on the disruption of cellular homeostasis and a deviation in the mechanisms that control cell fate. Chemoprevention is typically approached as an effort to minimize a cellular status associated with cancer initiation, such as oxidative stress or chronic inflammation [Lee Y M, Han S I, Song B C, & Yeum K J (2015) Bioactives in Commonly Consumed Cereal Grains: Implications for Oxidative Stress and Inflammation. J Med Food 18(11):1179-1186]. Proliferation, differentiation, quiescence, and apoptosis contain many levels of complexity which are often disrupted in cancer, leading to uncontrolled cellular growth. While there are many different mechanisms by which a cell undergoes malignant transformation, there is a set of conserved traits which are present. These so-called ‘hallmarks’ of cancer include abilities of uninhibited replication, promotion of angiogenesis, and evasion of growth suppression signaling [Dias M H, Kitano E S, Zelanis A, & Iwai L K (2016) Proteomics and drug discovery in cancer. Drug Discov Today 21(2):264-277; Matsumoto A, et al. (2016) Biological markers of invasive breast cancer. Jpn J Clin Oncol 46(2):99-105; Workman P (2001) New drug targets for genomic cancer therapy: successes, limitations, opportunities and future challenges. Curr Cancer Drug Targets 1(1):33-47]. For chemotherapeutics, the differences in these pathways can be exploited to target signaling pathways associated with growth, apoptosis, and the malignant transformation of a tumor. In terms of chemoprevention, cellular insults that disrupt homeostasis must be removed. Some of these are exogenous toxins and reactive species, which must be metabolized and excreted before damage ensues. Otherwise, endogenous cellular components can become compromised which disrupt signaling pathways, leading to various physiological problems and often resulting in neoplasia.

Scientific discoveries on the pathogenesis of chronic inflammatory diseases such as rheumatoid arthritis, Crohn's disease, psoriasis, psoriatic arthritis, and ankylosing spondylitis have revealed common mechanistic properties. Research has revealed that tumor necrosis factor (TNF) and interleukin-1 (IL1) are major players in the inflammatory response. Elevated levels of TNF at sights of inflammation have been associated with the pathogenesis of these chronic inflammatory diseases [Keifer J, et al. (1991) Transgenic mice expressing human tumour necrosis factor: a predictive genetic model of arthritis. EMBO J 10(13):4025-4031]. Cellular exposure to reactive oxygenated species and chemical toxicants can induce oxidative damage to DNA, proteins, and lipids. Oxidative impairment of cellular components has also been implicated in the development and progression of a wide array of diseases, including neurodegenerative diseases and cancer. To protect against insult and maintain cellular redox homeostasis, eukaryotes are equipped with an endogenous defence system comprised of a series of signaling cascades. One oxidative stress response is activation of the Nrf2-driven antioxidant response element (ARE), which leads to the induction of numerous cytoprotective phase II enzymes. Phase II enzymes are conjugating enzymes that function to make potentially harmful endogenous and exogenous compounds more water soluble, and thus more easily excreted. Some endogenous ligands include glutathione, glucuronic acid, and sulfate for which conjugation can be catalysed by glutathione S-transferases (GST), UDP-glucuronosyl transferases (UGTs), and sulfotransferases, respectively. Collectively, detoxification enzymes function by metabolizing and excreting harmful agents and by-products of oxidative stress. Interestingly, there is a substantial amount of cross talk between the ARE/Nrf2 and TNF/NFκB pathways. One of the key players in the crosstalk between these pathways is heme oxygenase 1 (HMOX1), which has been reported to inhibit the pro-inflammatory signals of NFκB, making NRF2 an attractive target for chemopreventive agents to combat both oxidative and inflammatory stresses.

While antioxidant activity is commonly associated with direct radical scavenging activity, an alternative way to increase the antioxidant status of a cell or body is to concertedly enhance the endogenous defense system consisting of antioxidant enzymes and detoxification enzymes, which presumably causes a more sustained, longer-lasting effect. Phase II and other antioxidant enzymes are commonly regulated by the antioxidant response element (ARE) on the transcriptional level [Kensler T W, Wakabayashi N, Biswal S. Cell survival responses to environmental stresses via the Keap1-Nrf2-ARE pathway. Annu. Rev. Pharmacol. Toxicol. 47:89-116; 2007]. Increased expression of these enzymes correlates with a decrease in cellular damage by radical oxygen species (ROS), which are implicated in inflammation and the pathogenesis of many disorders, including cancer, neurodegeneration, and aging [Chen X-L, Kunsch C. Induction of cytoprotective genes through Nrf2/antioxidant response element pathway: a new therapeutic approach for the treatment of inflammatory diseases. Curr. Pharm. Des. 10:879-891; 2004; Surh Y J. Cancer chemoprevention with dietary phytochemicals. Nat. Rev. Cancer 3:768-780; 2003; van Muiswinkel F L, Kuiperij H B. The Nrf2-ARE signaling pathway: promising drug target to combat oxidative stress in neurodegenerative disorders. Curr. Drug Targets CNS Neurol. Disord. 4:267-281; 2005]. In humans, the antioxidant response element (ARE) regulates the expression of cytoprotective antioxidant enzymes [e.g., heme oxygenase-1 (HMOX 1), glutathione-S-transferases (GSTs), NAD(P)H:quinone oxidoreductase 1 (NQO1)], which contribute to the endogenous defense against oxidative stress [Kensler T W, Wakabayashi N, Biswal S. Cell survival responses to environmental stresses via the Keap1-Nrf2-ARE pathway. Annu. Rev. Pharmacol. Toxicol. 47:89-116; 2007]. The major transcription factor involved in the induction of phase II enzymes is nuclear factor E2-related factor 2 (Nrf2), a Cap ‘n’ Collar (CNC) type basic region-leucine zipper (bZip) transcription factor that, upon activation by ARE inducers, translocates to the nucleus, binds to the ARE sequence as a heterodimer with one of the small bZip proteins, Mafs, and activates ARE-dependent genes. Nrf2 is negatively regulated by the cysteine-rich protein Keap1. Keap1 serves to sequester Nrf2 in the cytoplasm and interacts with Cul3-based E3 ubiquitin ligase to target Nrf2 for proteasomal degradation [Dinkova-Kostova A T, Holtzclaw W D, Kensler T W. The role of Keap1 in cellular protective responses. Chem. Res. Toxicol. 18:1779-1791; 2005; Kobayashi M, Yamamoto M. Nrf2-Keap1 regulation of cellular defense mechanisms against electrophiles and reactive oxygen species. Adv. Enzyme Regul. 46:113-140; 2006; Zhang D D. Mechanistic studies of the Nrf2-Keap1 signaling pathway. Drug. Metab. Rev. 38:769-789; 2006].

Nrf2 knockout mice show diminished detoxification capabilities, decreased responsiveness to chemoprotective agents, and enhanced susceptibility to oxidative stress induced cell death [Chan K, Han X-D, Kan Y W. An important function of Nrf2 in combating oxidative stress: detoxification of acetaminophen. Proc. Natl. Acad. Sci. USA 98:4611-4616; 2001; Ramos-Gomez M, Kwak M-K, Dolan P M, Itoh K, Yamamoto M, Talalay P, et al. Sensitivity to carcinogenesis is increased and chemoprotective efficacy of enzyme inducers is lost in nrf2 transcription factor-deficient mice. Proc. Natl. Acad. Sci. USA 98:3410-3415; 2001; Calkins M J, Jakel R J, Johnson D A, Chan K, Kan Y W, Johnson J A. Protection from mitochondrial complex II inhibition in vitro and in vivo by Nrf2-mediated transcription. Proc. Natl. Acad. Sci. USA 102:244-249; 2005]. Conversely, Nrf2 overexpression protects from oxidative stress [Chan K, Kan Y W, Johnson J A. Protection from mitochondrial complex II inhibition in vitro and in vivo by Nrf2-mediated transcription. Proc. Natl. Acad. Sci. USA 102:244-249; 2005]. NQ01-deficient individuals are at a considerably higher risk of developing leukemia following occupational exposure to benzene [Nebert D W, Roe A L, Vandale S E, Bingham E, Oakley G G. NAD(P)H:quinone oxidoreductase (NQO1) polymorphism, exposure to benzene, and predisposition to disease: a HuGE review. Genet. Med. 4:62-70; 2002]. The activation of the Nrf2-ARE pathway is a valid cancer preventive strategy, and sulforaphane, a constituent of broccoli, is an example of a cancer preventive natural product that acts through this mechanism [Surh Y J. Cancer chemoprevention with dietary phytochemicals. Nat. Rev. Cancer 3:768-780]. We hypothesized and preliminarily demonstrated that some seaweeds and algae are able to activate this signaling pathway and that some of the beneficial, particularly antioxidant, properties may be mediated through ARE activation as opposed to only direct scavenging properties [Wang R, Paul V J, Luesch H. Seaweed extracts and unsaturated fatty acid constituents from the green alga Ulva lactuca as activators of the cytoprotective Nrf2-ARE pathway. Free Rad. Biol. Med. doi10.1016/j.freeradbiomed.2012.12.019 (Epub Jan. 4, 2013); 2013].

ARE activation may also be particularly relevant to prostate cancer [Sikka S C. Role of oxidative stress response elements and antioxidants in prostate cancer pathobiology and chemoprevention—a mechanistic approach. Curr. Med. Chem. 10:2679-2692; 2003]. The most common hallmark in prostate cancer is the silencing of glutathione-S-transferase (GST)-π (GSTP1) due to DNA methylation, which is nearly universal [Lee W H, Morton R A, Epstein J I, Brooks J D, Campbell P A, Bova G S, et al. Cytidine methylation of regulatory sequences near the π-class glutathione S-transferase gene accompanies human prostatic carcinogenesis. Proc. Natl. Acad. Sci. USA 91:11733-11737; 1994; Lee W-H, Isaacs W B, Bova G S, Nelson W G. CG island methylation changes near the GSTP1 gene in prostatic carcinoma cells detected using the polymerase chain reaction: a new prostatic biomarker. Cancer Epidemiol. Biomark. Prev. 6:443-450; 1997; Lin X, Tascilar M, Lee W H, Vles W J, Lee B H, Veeraswamy R, et al. GSTP1 cpG island hypermethylation is responsible for the absence of GSTP1 expression in human prostate cancer cells. Am. J. Pathol. 159:1815-1826; 2001]. Because of the lack of GSTP1 expression in prostate cancer (regardless of grade or stage), induction of GSTs and other phase II enzymes through ARE activation is a promising prostate cancer-preventive strategy [Lee W H, Morton R A, Epstein J I, Brooks J D, Campbell P A, Bova G S, et al. Cytidine methylation of regulatory sequences near the π-class glutathione S-transferase gene accompanies human prostatic carcinogenesis. Proc. Natl. Acad. Sci. USA 91:11733-11737; 1994; Lee W-H, Isaacs W B, Bova G S, Nelson W G. CG island methylation changes near the GSTP1 gene in prostatic carcinoma cells detected using the polymerase chain reaction: a new prostatic biomarker. Cancer Epidemiol. Biomark Prev. 6:443-450; 1997; Lin X, Tascilar M, Lee W H, Vles W J, Lee B H, Veeraswamy R, et al. GSTP1 cpG island hypermethylation is responsible for the absence of GSTP1 expression in human prostate cancer cells. Am. J. Pathol. 159:1815-1826; 2001; Brooks J D, Paton V G, Vidanes G. Potent induction of phase 2 enzymes in human prostate cells by sulforaphane. Cancer Epidemiol. Biomark. Prev. 10:949-954; 2001; Brooks J D, Goldberg M F, Nelson L A, Wu D, Nelson W G. Identification of potential prostate cancer preventive agents through induction of quinone reductase in vitro. Cancer Epidemiol. Biomark. Prev. 11:868-875; 2002]. While prostate cancer is the second leading cause of cancer death in American men, prostate cancer is rarely diagnosed and contributes little to cancer mortality in Asia [Greenlee R T, Hill-Harmon M B, Murray T, Thun M. Cancer statistics, 2001. CA Cancer J. Clin. 51:15-36; 2001; Carter B S, Carter H B, Isaacs J T. Epidemiologic evidence regarding predisposing factors to prostate cancer. Prostate 16:187-197; 1990; Yu H, Harris R E, Gao Y T, Gao R, Wynder E L. Comparative epidemiology of cancers of the colon, rectum, prostate, and breast in Shanghai, China versus the United States. Int. J. Epidemiol. 20:76-81; 1991]. However, men migrating from Asia to the USA increase their risk, which remains elevated in their male descendants [Shimizu H, Ross R K, Bernstein L, Yatani R, Henderson B E, Mack T M. Cancers of the prostate and breast among Japanese and white immigrants in Los Angeles County. Br. J. Cancer 63:963-966; 1991; Whittemore A S, Kolonel L N, Wu A H, John E M, Gallagher R P, Howe G R, et al. Prostate cancer in relation to diet, physical activity, and body size in blacks, whites, and Asians in the United States and Canada. J. Natl. Cancer Inst. 87:652-661; 1995; Haenzel W, Kurihara M, Mortality from cancer and other diseases among Japanese men in the United States. J. Natl. Cancer Inst. 40:43-68; 1968; Danley K L, Richardson J L, Bernstein L, Langholz B, Ross R K. Prostate cancer: trends in mortality and stage-specific incidence rates by racial/ethnic group in Los Angeles County, California (United States). Cancer Cause Control 6:492-498; 1995]. While environmental factors may play a role, this observation may be attributable to lifestyle changes. Notably, diet in Asia largely includes seaweed, suggesting a possible connection between algae consumption and decreased prostate cancer risk. Many other diseases, including those with an inflammation component such as colon cancer, rectal cancer, stomach cancer, Crohn's Disease, irritable bowel syndrome (IBS), inflammatory bowel disease (IBD), rheumatoid arthritis, psoriasis, psoriatic arthritis, and ankylosing spondylitis, are caused by aberrant oxidative stress and may be prevented or interfered with via enhancing the cellular antioxidant status.

Marine algae (seaweeds) have been used as a food source and medicine for centuries [Chapman V J, Chapman D J. In Seaweeds and Their Uses. (Chapman and Hall, New York) pp 62-67; 1980]. This includes green algae (Chlorophyta), red algae (Rhodophyta) and brown algae (Ochrophyta). In addition, various edible seaweeds are high in nutritional value, providing minerals, proteins, vitamins, polysaccharides and antioxidants [Arasaki S & Arasaki T (1983) Low calorie, high nutrition vegetables from the sea. To help you look and feel better. (Japan Publications, Inc., Tokyo)]. Consumption of seaweed, which predominantly occurs in Japan, was found to be inversely related to various cancers, including colon, rectal and stomach cancer [Hoshiyama Y, Sekine T, Sasaba T. A case-control study of colorectal-cancer and its relation to diet, cigarettes, and alcohol-consumption in Saitama Prefecture, Japan. Tohoku J. Exp. Med. 171:153-165; 1993; Hoshiyama Y, Sasaba T. A case-control study of single and multiple stomach cancers in Saitama Prefecture, Japan. Jpn. J. Cancer Res. 83:937-943; 1992]. Seaweed is a major part of the Okinawan food culture, and Okinawans have the longest life expectancy in the world and low disability rates [Sho H. History and characteristics of Okinawan longevity food. Asia Pac. J. Clin. Nutr. 10:159-164; 2001]. Numerous beneficial properties of algal extracts and constituents have been reported, however, usually only in a descriptive manner, without pinpointing specific bioactive components or invoking specific molecular pathways. Other anecdotal evidence of health-benefits include greater life expectancy and low disability rates [Sho H (2001) History and characteristics of Okinawan longevity food. Asia Pac J Clin Nutr 10(2):159-164]. Seaweeds and their constituents have been linked to beneficial activities, including recent reports that demonstrated anti-oxidant and life expansion activities of algal extracts [Ratnayake R, Liu Y, Paul V J, & Luesch H (2013) Cultivated sea lettuce is a multiorgan protector from oxidative and inflammatory stress by enhancing the endogenous antioxidant defense system. Cancer Prev Res (Phila) 6(9):989-999; Snare D J, Fields A M, Snell T W, & Kubanek J (2013) Lifespan extension of rotifers by treatment with red algal extracts. Exp Gerontol 48(12):1420-1427; Wang R, et al. (2013) In vitro and in vivo characterization of a tunable dual-reactivity probe of the Nrf2-ARE pathway. ACS Chem Biol 8(8):1764-1774; Wang R, Paul V J, & Luesch H (2013) Seaweed extracts and unsaturated fatty acid constituents from the green alga Ulva lactuca as activators of the cytoprotective Nrf2-ARE pathway. Free Radic Biol Med 57:141-153].

Cymopolia barbata is a green marine alga commonly found in the shallow coastal waters near the Florida Keys. Molecules collectively referred to as cymopols were among the first halogenated natural products derived from green algae [Hogberg H-E and Thomson R J (1976) The cymopols, a group of prenylated bromohydroquinones from the green calcareous alga Cymopolia J Chem Soc Perkin 1 (16) 1696-1701]. These molecules, containing a bromohydroquinone motif, have since been associated with various bioactivities: antimutagenic [Wall M E, Wani M C, Manikumar G, Taylor H, Hughes T J, Gaetano K. Plant antimutagenic agents (1989) 7(1) structure and antimutagenic properties of cymbarbatol and 4-isocymobarbatol, new cymopols from green alga (Cymopolia barba). J Nat Products 52 (5):1092-1099], phospholipase A2 inhibition [Mayer A M S, Paul V J, Fenical W, Norris J N, de Carvalho M S, Jacobs R S (1993) Phospholipase A2 inhibitors from marine algae. Hydrobiologia 260/261:521-29], inhibition of LFA-1/ICAM-1 mediated cell adhesion [Takamatsu S, Hodges T W, Rajbhandari I, Gerwick W H, Hamann M T, Nagle D G. Marine natural products as novel antioxidant prototypes. J. Nat. Prod. 2002; 66:605-608], antifungal and antimicrobial [Martínez-Nadal N G, Rodríguez LV, Casillas S (1964) Isolation and characterization sarganin complex, a new broad spectrum antibiotic isolated from marine algae. Antimicrob Agents Chemother 10:13], as well as anti-oxidative properties by way of free-radical sequestration, as determined by the 2,2-diphenyl-1-picrylhydrazyl, or DPPH assay [Takamatsu S, Hodges T W, Rajbhandari I, Gerwick W H, Hamann M T, Nagle D G. Marine natural products as novel antioxidant prototypes. J. Nat. Prod. 2002; 66:605-608].

Many naturally occurring small molecule inducers of the Nrf2-ARE pathway have been identified and explored as chemopreventive or therapeutic agents. For example, curcumin [Balogun, E.; Hogue, M.; Gong, P.; Killeen, E.; Green, C. J.; Foresti, R.; Alam, J.; Motterlini, R. Curcumin activates the haem oxygenase-1 gene via regulation of Nrf2 and the antioxidant-responsive element. Biochem J. 371:887-95; 2003], the active ingredient in traditional herbal remedy and dietary spice turmeric (Curcuma longa) is currently in clinical trials for multiple conditions, including several cancers and Alzheimer's disease [Hatcher, H.; Planalp, R.; Cho, J.; Torti, F. M.; Torti, S. V. Curcumin: from ancient medicine to current clinical trials. Cell Mol Life Sci. 65:1631-52; 2008]. The skin of red grapes (Vitis vinifera) is rich in resveratrol [Langcake, P.; Pryce, R. J. Production of Resveratrol by Vitis-Vinifera and Other Members of Vitaceae as a Response to Infection or Injury. Physiological Plant Pathology. 9:77-86; 1976; Rubiolo, J. A.; Mithieux, G.; Vega, F. V. Resveratrol protects primary rat hepatocytes against oxidative stress damage: activation of the Nrf2 transcription factor and augmented activities of antioxidant enzymes. Eur J Pharmacol. 591:66-72; 2008], which was found to be responsible for an inverse relationship between grape consumption and breast cancer occurrence in an epidemiologic study [Levi, F.; Pasche, C.; Lucchini, F.; Ghidoni, R.; Ferraroni, M.; La Vecchia, C. Resveratrol and breast cancer risk. Eur J Cancer Prev. 14:139-42; 2005]. In a clinical setting, resveratrol was observed to induce the re-expression of tumor suppressor genes in a group of women who are at increased risk of breast cancer [Zhu, W.; Qin, W.; Zhang, K.; Rottinghaus, G. E.; Chen, Y. C.; Kliethermes, B.; Sauter, E. R. Trans-resveratrol alters mammary promoter hypermethylation in women at increased risk for breast cancer. Nutr Cancer. 64:393-400; 2012]. The detoxification enzyme inducer, sulforaphane [Kensler, T. W.; Egner, P. A.; Agyeman, A. S.; Visvanathan, K.; Groopman, J. D.; Chen, J. G.; Chen, T. Y.; Fahey, J. W.; Talalay, P. Keap1-Nrf2 Signaling: A Target for Cancer Prevention by Sulforaphane. Top Curr Chem. 2012], was found in many cruciferous vegetables. It has been shown that a daily regimen of hot water infused with 3-day-old broccoli sprouts has promising results in cancer chemoprevention in healthy individuals [Kensler, T. W.; Chen, J. G.; Egner, P. A.; Fahey, J. W.; Jacobson, L. P.; Stephenson, K. K.; Ye, L.; Coady, J. L.; Wang, J. B.; Wu, Y.; Sun, Y.; Zhang, Q. N.; Zhang, B. C.; Zhu, Y. R.; Qian, G. S.; Carmella, S. G.; Hecht, S. S.; Benning, L.; Gange, S. J.; Groopman, J. D.; Talalay, P. Effects of glucosinolate-rich broccoli sprouts on urinary levels of aflatoxin-DNA adducts and phenanthrene tetraols in a randomized clinical trial in He Zuo township, Qidong, People's Republic of China. Cancer Epidemiol Biomarkers Prev. 14:2605-13; 2005]. Broccoli sprouts (Brassica oleracea italica) contain high levels of its precursor, glucoraphanin [Farnham, M. W.; Stephenson, K. K.; Fahey, J. W. Glucoraphanin level in broccoli seed is largely determined by genotype. Hortscience. 40:50-53; 2005], which can be enzymatically converted to sulforaphane in the gastrointestinal tract after ingestion [Zhang, Y.; Talalay, P.; Cho, C. G.; Posner, G. H. A major inducer of anticarcinogenic protective enzymes from broccoli: isolation and elucidation of structure. Proc Natl Acad Sci USA. 89:2399-403; 1992].

The marine environment has also proven to be a rich source of potent compounds with diverse therapeutic properties [Newman, D. J.; Cragg, G. M. Marine natural products and related compounds in clinical and advanced preclinical trials. J Nat Prod. 67:1216-38; 2004; Montaser, R.; Luesch, H. Marine natural products: a new wave of drugs? Future Med Chem. 3:1475-89; 2011]. For example, several molecules with anti-cancer activities based on leads from marine cyanobacteria have been described [Taori, K.; Paul, V. J.; Luesch, H. Structure and activity of largazole, a potent antiproliferative agent from the Floridian marine cyanobacterium Symploca sp. J Am Chem Soc. 130:1806-7; 2008-20; Hong, J.; Luesch, H. Largazole: from discovery to broad-spectrum therapy. Nat Prod Rep. 29:449-56; 2012; Chen, Q. Y.; Liu, Y.; Luesch, H. Systematic Chemical Mutagenesis Identifies a Potent Novel Apratoxin A/E Hybrid with Improved in Vivo Antitumor Activity. ACS Med Chem Lett. 2:861-865; 2011]. Additionally, the free radical scavenger fucoxanthin, a carotenoid from a common edible seaweed, Hijikia fusiformis [Yon, X.; Chuda, Y.; Suzuki, M.; Nagata, T. Fucoxanthin as the major antioxidant in Hijikia fusiformis, a common edible seaweed. Biosci Biotechnol Biochem. 63:605-7; 1999], was found to activate the antioxidant defense system (Nrf2/ARE) in mouse liver cells.

The microbiota is an integral part of the host and uniquely contributes to various biological activities [Perez-Chanona E, Muhlbauer M, & Jobin C (2014) The microbiota protects against ischemia/reperfusion-induced intestinal injury through nucleotide-binding oligomerization domain-containing protein 2 (NOD2) signaling. Am J Pathol 184(10:2965-2975]. Importantly, microbial composition and activity are influenced by various conditions such as inflammation, infection, antibiotic treatment and diet [David L A, et al. (2014) Diet rapidly and reproducibly alters the human gut microbiome. Nature 505(7484):559-563]. While certain colon intestinal bacteria including E. coli carrying the genotoxin pks (e.g., NC101) are known to cause colorectal cancer (CRC) (23), other bacteria have beneficial interactions with the host, including some that modulate host Nrf2 signaling. Thus, it is prudent to take the microbiome into account when investigating the effect of seaweed or algae as a diet or oral preparation, particularly since it is known that certain seaweeds have antimicrobial activity, including Cymopolia barbata, and we investigated the change in microbiome composition in response to short-term consumption of this seaweed by mice.

However, despite these developments, there exists an unmet need for additional antioxidants and for additional treatments for ROS-mediated diseases. This study follows on previous research that showed that extracts of marine algae can activate the Nrf2-ARE pathway, and that extracts of Ulva spp. were particularly active among a variety of seaweeds tested [Wang R, Paul V J, Luesch H. Seaweed extracts and unsaturated fatty acid constituents from the green alga Ulva lactuca as activators of the cytoprotective Nrf2-ARE pathway. Free Rad. Biol. Med; doi10.1016/j.freeradbiomed.2012.12.019 (Epub Jan. 4, 2013); 2013]. As a result of ongoing investigations to identify new drug leads from marine sources, we report seaweed extract compositions isolated from cultivated green alga Cymopolia barbata, processes for isolation, enriched active fractions, and isolated active agents. The extracts, enriched active extracts, and compounds herein are found to be activators of the cytoprotective Nrf2-ARE pathway. These findings provide new alternatives for the treatment and/or prevention of reactive oxygen species (ROS)-mediated diseases and diseases alleviated or prevented through the activation of the Nrf2-ARE (antioxidant response element) pathway, such as inflammation (e.g., Crohn's Disease, irritable bowel syndrome (IBS), inflammatory bowel disorder (IBD), rheumatoid arthritis, psoriasis, psoriatic arthritis, ankylosing spondylitis), cancer (e.g., colon cancer, stomach cancer, rectal cancer, prostate cancer), Alzheimer's disease and other neurodegenerative disorders, stroke, chronic kidney disease, type II diabetes, and aging itself.

### BRIEF SUMMARY OF THE INVENTION

This invention is directed towards seaweed extract compositions, enriched active extracts, processes for isolation, isolated active agents, and methods of treating and/or preventing disease, disorders and conditions in a subject, including, reactive oxygen species (ROS)-mediated diseases and diseases alleviated or prevented through the activation of the Nrf2-ARE (antioxidant response element) pathway, including proliferative diseases and disorders (e.g., colon cancer, stomach cancer, rectal cancer, prostate cancer), inflammation (e.g., Crohn's Disease, irritable bowel syndrome (IBS), inflammatory bowel disorder (IBD), rheumatoid arthritis, psoriasis, psoriatic arthritis, ankylosing spondylitis), Alzheimer's disease and other neurodegenerative disorders, stroke, chronic kidney disease, type II diabetes, and certain diseases and disorders of aging and associated with aging and exposure, by use of the extracts, enriched active extracts, compounds, and compositions thereof.

This invention is directed towards seaweed extract compositions, enriched active extracts, processes for isolation, isolated active agents, methods for activating the Nrf2-ARE pathway, and methods of treating and/or preventing reactive oxygen species (ROS)-mediated diseases and diseases alleviated or prevented through the activation of the Nrf2-ARE (antioxidant response element) pathway, including proliferative diseases and disorders (e.g., colon cancer, stomach cancer, rectal cancer, prostate cancer), inflammation (e.g., Crohn's Disease, irritable bowel syndrome (IBS), inflammatory bowel disorder (IBD), rheumatoid arthritis, psoriasis, psoriatic arthritis, ankylosing spondylitis), cancer, Alzheimer's disease and other neurodegenerative disorders, stroke, chronic kidney disease, type II diabetes, and aging itself.

Another aspect of this invention is a composition comprising a seaweed extract herein (e.g., extract of Cymopolia sp.) for systemic, topical and/or ocular administration. Another aspect is a composition comprising an enriched active extract from a seaweed extract herein for systemic, topical and/or ocular administration. Another aspect is a composition comprising an isolated compound and/or isolated compound mixture from a seaweed extract herein for systemic, topical and/or ocular administration.

In one embodiment, the compound (or combinations of compounds) delineated herein is obtained from a procedure comprising extraction from seaweed. In certain embodiments, the procedure for use in obtaining the compound (or combinations of compounds) further includes any of isolation, enrichment, evaporation, and partitioning steps of the seaweed extracts.

Another aspect of this invention is a pharmaceutical composition comprising a seaweed extract herein or a compound that occurs in a seaweed extract herein.

In one embodiment, the invention provides an extract from seaweed isolated by:
a). Exposing said algae to a solvent or solvent combination;
b). Filtering the material/mixture from step a); and
c). Removing the solvent or solvent combination from step b).

In another embodiment, the invention provides an extract from seaweed isolated by:
a) Exposing said algae to a solvent or solvent combination;
b) Filtering the material/mixture from step a);
c) Removing the solvent or solvent combination from step b);
d) Purifying the material/mixture from step c); and
e) Removing the chromatography mobile phase to provide enriched fractions.

Another aspect is where the concentrated fractions are screened in an ARE reporter assay. Another aspect is where the solvent or solvent combination in extraction step a) is selected from the group consisting of ethyl acetate, methanol, hexanes, ethanol, isopropanol, acetonitrile, water, and dichloromethane. Another aspect is where the solvent or solvent combination in extraction step a) is ethyl acetate. Another aspect is where the solvent or solvent combination in extraction step a) includes ethyl acetate. Another aspect is where steps a)-c) are repeated with the same or different solvent or solvent combination as used in the previous iteration(s). Another aspect is where the seaweed is the green alga Cymopolia sp. Another aspect is where the green alga Cymopolia sp. is cultivated.

Another aspect is a compound or extract obtained by one or more steps of the processes or procedures delineated herein, including specifically as delineated in the Examples herein.

Another aspect is wherein the algae comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

Another aspect is wherein the seaweed extract comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

Another aspect is where the seaweed extract comprises (E)-2-bromo-5-(3,7-dimethylocta-2,6-dien-1-yl)benzene-1,4-diol (Cymopol, 1).

Another aspect is where the seaweed extract is enriched in one or more compounds selected from the group consisting of:

a) E)-2-bromo-5-(3,7-dimethylocta-2,6-dien-1-yl)benzene-1,4-diol (Cymopol, 1);

b) (E)-2-bromo-5-(7-hydroxy-3,7-dimethyloct-2-en-1-yl)benzene-1,4-diol (7-hydroxycymopol, 2);

c) 2,6-dibromo-1,1,4a-trimethyl-2,3,4,4a,9,9a-hexahydro-1H-xanthen-7-ol (Cymobarbatol, 3); and

d) 5-bromo-24(3-bromo-2,2-dimethyl-6-methylenecyclohexyl)methyl)-4-methoxyphenol (Cyclocymopol monomethyl ether, 4).

Another aspect is where the seaweed extract is enriched in (E)-2-bromo-5-(3,7-dimethylocta-2,6-dien-1-yl)benzene-1,4-diol (Cymopol, 1).

In another aspect, the invention provides a pharmaceutical composition comprising a seaweed extract and/or compound(s) isolated therefrom and a pharmaceutically acceptable carrier. In another aspect, the extract comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

In another aspect, the invention provides a pharmaceutical composition comprising an enriched seaweed extract (e.g., enriched through evaporation, enriched through fractionation, enriched through partial purification) and a pharmaceutically acceptable carrier.

In another aspect, the invention provides a pharmaceutical composition comprising an isolated compound or isolated compound mixture obtained from a seaweed/algae using any of the processes delineated herein. In another aspect, the pharmaceutical composition comprises two or more isolated compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4);

and a pharmaceutically acceptable carrier.

In another aspect, the invention provides a composition for topical administration comprising any algal extract delineated herein, and a lotion, cream, or ointment carrier. In another aspect, the extract comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

In another aspect, the invention provides a composition for topical administration comprising any algal enriched extract delineated herein, and a lotion, cream, or ointment carrier. In another aspect, the extract is enriched in one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

In another aspect, the invention provides a composition for topical administration comprising one or more isolated compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

In another aspect, the invention provides a composition for ocular administration comprising any algal extract delineated herein, and one or more carriers or diluents suitable for ocular administration. In another aspect, the extract comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

In another aspect, the invention provides a composition for ocular administration comprising any algal enriched extract delineated herein, and one or more carriers or diluents suitable for ocular administration. In another aspect, the extract is enriched in one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

In another aspect, the invention provides a composition for ocular administration comprising one or more isolated compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4);

and one or more carriers or diluents suitable for ocular administration.

In other aspects, the invention provides a method of treating and/or preventing a disease, disorder, or symptom thereof in a subject, comprising administering to the subject any compound, seaweed extract, or enriched seaweed extract herein. In another aspect, the compound, seaweed extract, or enriched seaweed extract is administered in an amount and under conditions sufficient to ameliorate the disease, disorder, or symptom thereof in a subject. In another aspect, the disease, disorder, or symptom includes proliferative diseases and disorders, inflammation (e.g., Crohn's Disease, irritable bowel syndrome (IBS), inflammatory bowel disease (IBD), rheumatoid arthritis, psoriasis, psoriatic arthritis, or ankylosing spondylitis), cancer (e.g., colon cancer, rectal cancer, stomach cancer, or prostate cancer), Alzheimer's disease and other neurodegenerative disorders, stroke, chronic kidney disease, type II diabetes, cancer, tumor growth, cancer of the colon, breast, bone, brain and others (e.g., osteosarcoma, neuroblastoma, colon adenocarcinoma), cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, cutaneous T-cell lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma). Other diseases and disorders that can be treated include the treatment of inflammatory disorders, neurodegenerative diseases, protozoal and latent viral infections, and (fibro)proliferative disorders, and aging itself.

In other aspects, the invention provides a method of modulating Nrf2-ARE activity in a subject, comprising contacting the subject with any compound, seaweed extract, or enriched seaweed extract herein, in an amount and under conditions sufficient to modulate Nrf2-ARE activity. In another aspect, the modulation is activation.

In other aspects, the invention provides a method of modulating the proliferation activity in a subject, comprising contacting the subject with any compound, seaweed extract, or enriched seaweed extract herein, in an amount and under conditions sufficient to modulate proliferation activity.

In one aspect, the invention provides a method of treating a subject suffering from or susceptible to a proliferation related disorder or disease, comprising administering to the subject an effective amount of a compound, seaweed extract, or enriched seaweed extract or pharmaceutical composition of any compound, seaweed extract, or enriched seaweed extract herein. In another aspect, the proliferation disease or disorder is cancer. In another aspect, the cancer is colon cancer, rectal cancer, stomach cancer, or prostate cancer.

In one aspect, the invention provides a method of treating a subject suffering from or susceptible to an inflammatory disorder or disease, comprising administering to the subject an effective amount of a compound, seaweed extract, or enriched seaweed extract or pharmaceutical composition of any compound, seaweed extract, or enriched seaweed extract herein. In another aspect, the inflammatory disease or disorder is Crohn's Disease, irritable bowel syndrome (IBS), inflammatory bowel disease (IBD), rheumatoid arthritis, psoriasis, psoriatic arthritis, or ankylosing spondylitis.

In another aspect, the invention provides a method of treating a subject suffering from or susceptible to a ROS-mediated disorder or disease, comprising administering to the subject an effective amount of a compound, seaweed extract, or enriched seaweed extract or pharmaceutical composition of any compound, seaweed extract, or enriched seaweed extract herein.

In another aspect, the invention provides a method of treating a subject suffering from or susceptible to a disorder or disease alleviated or prevented through the Nrf2-ARE pathway, comprising administering to the subject an effective amount of a compound, seaweed extract, or enriched seaweed extract or pharmaceutical composition of any compound, seaweed extract, or enriched seaweed extract herein. Another aspect is where the disorder or disease alleviated or prevented through the Nrf2-ARE pathway includes proliferative diseases and disorders, inflammation (e.g., Crohn's Disease, irritable bowel syndrome (IBS), inflammatory bowel disease (IBD), rheumatoid arthritis, psoriasis, psoriatic arthritis, or ankylosing spondylitis), cancer (e.g., colon cancer, rectal cancer, stomach cancer, or prostate cancer), Alzheimer's disease and other neurodegenerative disorders, stroke, chronic kidney disease, type II diabetes, cancer, tumor growth, cancer of the colon, breast, bone, brain and others (e.g., osteosarcoma, neuroblastoma, colon adenocarcinoma), cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, cutaneous T-cell lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma). Other diseases and disorders that can be treated include the treatment of inflammatory disorders, neurodegenerative diseases, protozoal and latent viral infections, (fibro)proliferative disorders, and aging itself.

In another aspect, the invention provides a method of treating a subject suffering from or susceptible to a proliferation related activity related disorder or disease, wherein the subject has been identified as in need of treatment for a proliferation related disorder or disease, comprising administering to said subject in need thereof, an effective amount of a compound, seaweed extract, or enriched seaweed extract or pharmaceutical composition of any compound, seaweed extract, or enriched seaweed extract herein, such that said subject is treated for said disorder.

In another aspect, the invention provides a method of treating a subject suffering from or susceptible to a ROS activity related disorder or disease, wherein the subject has been identified as in need of treatment for a ROS-related disorder or disease, comprising administering to said subject in need thereof, an effective amount of a compound, seaweed extract, or enriched seaweed extract or pharmaceutical composition of any compound, seaweed extract, or enriched seaweed extract herein, such that said subject is treated for said disorder.

In another aspect, the invention provides a method of treating a subject suffering from or susceptible to a Nrf2-ARE activity related disorder or disease, wherein the subject has been identified as in need of treatment for a Nrf2-ARE related disorder or disease, comprising administering to said subject in need thereof, an effective amount of a compound, seaweed extract, or enriched seaweed extract or pharmaceutical composition of any compound, seaweed extract, or enriched seaweed extract herein, such that said subject is treated for said disorder. Another aspect is where the said disorder includes proliferative diseases and disorders, inflammation (e.g., Crohn's Disease, irritable bowel syndrome (IBS), inflammatory bowel disease (IBD), rheumatoid arthritis, psoriasis, psoriatic arthritis, or ankylosing spondylitis), cancer (e.g., colon cancer, rectal cancer, stomach cancer, or prostate cancer), Alzheimer's disease and other neurodegenerative disorders, stroke, chronic kidney disease, type II diabetes, and aging itself.

In another aspect, the invention provides a method of treating a subject suffering from or susceptible to a cell proliferation related disorder or disease, wherein the subject has been identified as in need of treatment for a cell proliferation related disorder or disease, comprising administering to said subject in need thereof, an effective amount of a compound, seaweed extract, or enriched seaweed extract or pharmaceutical composition of any compound, seaweed extract, or enriched seaweed extract herein, such that cell proliferation in said subject is modulated (e.g., down regulated). In another aspect, the compound, seaweed extract, or enriched seaweed extract delineated herein preferentially targets cancer cells over nontransformed cells.

In a specific aspect, the invention provides a method of treating and/or preventing cancer (e.g., colon cancer, rectal cancer, stomach cancer, or prostate cancer), tumor growth, cancer of the colon, breast, bone, brain and others (e.g., osteosarcoma, neuroblastoma, colon adenocarcinoma), comprising administering to said subject in need thereof, an effective amount of any compound, seaweed extract, or enriched seaweed extract delineated herein, and pharmaceutically acceptable salts thereof. Other cancers that may be treated and/or prevented by the compositions and methods of the invention include cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, cutaneous T-cell lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma). Other diseases and disorders that can be treated and/or prevented include the treatment of inflammatory disorders (e.g., Crohn's Disease, irritable bowel syndrome (IBS), inflammatory bowel disease (IBD), rheumatoid arthritis, psoriasis, psoriatic arthritis, or ankylosing spondylitis), neurodegenerative diseases, protozoal and latent viral infections, and (fibro)proliferative disorders.

In a specific aspect, the invention provides a method of treating and/or preventing inflammation, Alzheimer's disease and other neurodegenerative disorders, stroke, chronic kidney disease, type II diabetes, aging itself, and other diseases mediated through ROS, comprising administering to said subject in need thereof, an effective amount of any compound, seaweed extract, or enriched seaweed extract delineated herein, and pharmaceutically acceptable salts thereof. In another aspect, the inflammatory disease or disorder is Crohn's Disease, irritable bowel syndrome (IBS), inflammatory bowel disease (IBD), rheumatoid arthritis, psoriasis, psoriatic arthritis, or ankylosing spondylitis.

In another aspect, the invention provides a method of treating and/or preventing diseases, disorders, or symptoms thereof mediated by activation of the Nrf2-ARE pathway in a subject in need thereof comprising administering to said subject, an effective amount of any compound, seaweed extract, or enriched seaweed extract delineated herein, and pharmaceutically acceptable salts thereof.

In another aspect, the invention provides a method of altering the microbiome in the gastrointestinal tract of a subject, comprising administering to the subject an effective amount of a compound, seaweed extract, or enriched seaweed extract or pharmaceutical composition of any compound, seaweed extract, or enriched seaweed extract herein.

In another aspect, the invention provides a method of improving the overall health of a subject, comprising administering to the subject an effective amount of a compound, seaweed extract, or enriched seaweed extract or pharmaceutical composition of any compound, seaweed extract, or enriched seaweed extract herein.

In another aspect, the invention provides a method of supplementing diet in a subject, comprising administering to the subject an effective amount of a compound, seaweed extract, or enriched seaweed extract or pharmaceutical composition of any compound, seaweed extract, or enriched seaweed extract herein.

In another aspect, the invention provides a method of manufacturing a dietary supplement comprising combining a compound, seaweed extract, or enriched seaweed extract or pharmaceutical composition of any compound, seaweed extract, or enriched seaweed extract herein with a carrier. In another aspect, the carrier is suitable for oral administration.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

### BRIEF DESCRIPTION OF THE DRAWINGS

The present invention is further described below with reference to the following non-limiting examples and with reference to the following figures, in which:

FIGS. 1A-1B. depict the tabulated ¹H NMR, ¹³C, COSY, and HMBC data for Cymopol (1), 7-hydroxy cymopol (2), Cymobarbatol (3), and Cyclocymopol monomethyl ether (4).

FIG. 2. depicts the ¹H NMR spectrum of (E)-2-bromo-5-(3,7-dimethylocta-2,6-dien-1-yl)benzene-1,4-diol (1) in CDCl₃.

FIG. 3. depicts the ¹³C NMR spectrum of (E)-2-bromo-5-(3,7-dimethylocta-2,6-dien-1-yl)benzene-1,4-diol (1) in CDCl₃.

FIG. 4. depicts the ¹H NMR spectrum of (E)-2-bromo-5-(7-hydroxy-3,7-dimethyloct-2-en-1-yl)benzene-1,4-diol (2) in CDCl₃.

FIG. 5. depicts the ¹³C NMR spectrum of (E)-2-bromo-5-(7-hydroxy-3,7-dimethyloct-2-en-1-yl)benzene-1,4-diol (2) in CDCl₃.

FIG. 6. depicts the ¹H NMR spectrum of 2,6-dibromo-1,1,4a-trimethyl-2,3,4,4a,9,9a-hexahydro-1H-xanthen-7-ol (3) in CDCl₃.

FIG. 7. depicts the ¹³C NMR spectrum of 2,6-dibromo-1,1,4a-trimethyl-2,3,4,4a,9,9a-hexahydro-1H-xanthen-7-ol (3) in CDCl₃.

FIG. 8. depicts the ¹H NMR spectrum of 5-bromo-24(3-bromo-2,2-dimethyl-6-methylenecyclohexyl)methyl)-4-methoxyphenol (4) in CDCl₃.

FIG. 9. depicts the ¹³C NMR spectrum of 5-bromo-24(3-bromo-2,2-dimethyl-6-methylenecyclohexyl)methyl)-4-methoxyphenol (4) in CDCl₃.

FIGS. 10A-10D. depict the FIG. 10A) chemical structures of cymopol (1), 7-hydroxy cymopol (2), cymobarbatol (3), cyclocymopol monomethyl ether (4); and increases in ARE-luciferase reporter gene transcription in FIG. 10B) LNCaP cell line; FIG. 10C) IMR-32 cell line; and FIG. 10D) Pgst::GFP reporter gene in C. elegans.

FIGS. 11A-11E. depict the ARE-mediated inductive effects of cymopols 1-4 and Cymopolia sp. non-polar extract (NP extract) in FIG. 11A) and FIG. 11C) IMR-32 cell line; FIG. 11B) LNCaP cell line; and an increase in FIG. 11D) NQO1 and FIG. 11E) GSH levels.

FIGS. 12A-12B. depict FIG. 12A) the profiling of Compound 1 and the non-polar extract (NP extract) in wild type and Nrf2 −/− cell lines; and FIG. 12B) the effect of Compound 1 and the non-polar extract (NP extract) in Nrf2 translocation in IMR-32 cells.

FIGS. 13A-13D. depict the reduction of FIG. 13A) PGE2; FIG. 13B) iNOS; and FIG. 13C) Cox2 with increasing FIG. 13D) Nqo1 levels by Compound 1 and Cymopolia sp. non-polar extract (NP extract) in an IFN-gamma macrophage model.

FIGS. 14A-14B. depict the effects of Compound 1 and Cymopolia sp. non-polar extract (NP extract) on Nqo1 gene transcript levels in various mouse organs.

FIGS. 15A-15B. depict the effects of Compound 1 and Cymopolia sp. non-polar extract (NP extract) on Hmox1 gene transcript levels in various mouse organs.

FIGS. 16A-16B. depict the activation profiles of various large intestinal pathways by FIG. 16A) Compound 1; and FIG. 16B) curcumin.

FIG. 17. depicts the colorectal cancer metastasis signaling pathway.

FIG. 18. depicts the top relevant cancer pathways (green fill=downregulated; red fill=upregulated; outline of black=cancer functionality, outline of blue=abdominal cancer functionality, outline of red=both general cancer and abdominal cancer functionalities).

FIG. 19. depicts the profiling of Compound 1 and the non-polar extract (NP extract) in MEFs containing Nrf2 −/− or Keap1 −/− mutations.

FIG. 20. depicts a silver catalyzed oxidation reaction of compound 1 to form the corresponding cymopol quinone (5).

FIG. 21. depicts the assessment of mRNA levels of the Nrf2 target gene NQ01 in IMR-32 cells after a 12 h treatment with cymopol 1 (hydroquinone) and its corresponding quinone 5, alongside structurally related positive control tBHQ and its active quinone metabolite, tBQ.

FIG. 22. depicts the knockdown efficiency of the siNRF2 in IMR-32 cells after 48 h.

FIG. 23. depicts the measurement of iNOS transcript levels, NO production, Cox2 transcript levels, PGE2 production, and levels of Nqo1 mRNA in RAW264.7 macrophage cells pretreated with various doses of compound 1 or NP extract prior to activation with inflammatory activating IFN-γ.

FIG. 24. depicts the treatment of wildtype MEFs, Nrf2−/−, and Keap1−/− MEF cells with Compound 1 and the NP extract. Compound 1 and NP extract were able to restore basal level of NO in wildtype MEFs, while Nrf2−/− and Keap1−/− MEF cells were resistant towards the anti-inflammatory properties of the cymopols.

FIG. 25. depicts the decrease of neutrophils to the cut site of the Tg(mpx::GFP)ill4 zebrafish line after treatment with Compound 1 and the NP extract.

FIG. 26. depicts an increase in ARE-driven gene transcription and a decrease in select NFκB driven genes after treating wildtype zebrafish embryos of the same age with Compound 1 and the NP extract.

FIG. 27. depicts a heatmap summary of mice given cymopol 1 or NP extract via oral gavage, resulting in elevated transcript levels of Nqo1 in various organs, particularly in the digestive tract.

FIG. 28. depicts a heatmap summary of mice given cymopol 1 or NP extract via oral gavage, resulting in elevated transcript levels of Hmox1 in various organs, particularly in the digestive tract.

FIG. 29. depicts the induction of Nqo1 in cecum and large intestines (* indicates p-value <0.05; ** indicates p-value <0.005; *** indicates p-values <0.0005).

FIG. 30. depicts the induction of Hmox1 in cecum and large intestines (* indicates p-value <0.05; ** indicates p-value <0.005; *** indicates p-values <0.0005).

FIG. 31. depicts the reduction of the DSS-induced inflammatory marker Lipocalin 2 (Lcn-2) by the NP extract.

FIG. 32. depicts the RNA-seq based analysis of the microbiome shift in the large intestines between control and low groups, PCoA1 FDR P<0.0001.

FIG. 33. depicts the RNA-seq based analysis of the microbiome shift in the large intestines between control and high groups, PCoA1 FDR P<0.05.

FIG. 34. depicts the RNA-seq based analysis of the microbiome shift in the large intestines between high and low groups, PCoA1 FDR P<0.0001.

FIG. 35. depicts the heatmap showing the mean log₁₀ normalized relative abundances of genera that were significantly different (FDR P<0.05) between control and low groups.

FIG. 36. depicts the heatmap showing the mean log₁₀ normalized relative abundances of genera that were significantly different (FDR P<0.05) between control and high groups.

FIG. 37. depicts the heatmap showing the mean log₁₀ normalized relative abundances of genera that were significantly different (FDR P<0.05) between high and low groups.

FIGS. 38A-38B. depict the reactivity of Compound 1 and NP extract towards Keap1 Cys-151 in cells transfected with mock cDNA-RFP, Keap1-CBD, or Keap1-C151S-CBD and HA-Cul3. Compound 1 and NP extract increase binding of Keap1-Cul3 interaction in a dose response manner. This effect enhanced in the absence of Cys-151, indicating the role of this residue in Keap1-Cul3 interaction. In the whole cell lysate, it is seen that compound 1 and NP extract increase dimerization, and that the effect is decreased in Keap1-C151S, suggesting that cymopols promote dimerization of Keap1 via Cys-151.

FIGS. 39A-39B. depict the reactivity of Compound 1 and NP extract towards Keap1 Cys-151 in cells transfected with mock cDNA-RFP, Keap1-CBD, or Keap1-C151S-CBD and HA-Nrf2. Compound 1 and NP increase dimerization of Keap1 in a dose-response manner. The whole cell lysates indicate that this is dependent on Cys-151, as the mutants do not have an increased dimerization.

FIGS. 40A-40B. depict the reactivity of Compound 1 and NP extract towards Keap1 Cys-151 in cells transfected with mock cDNA-RFP, Keap1-CBD, or Keap1-C151S-CBD and Gal4-Neh2 and HA-Ub.

FIG. 41. depicts Nqo1 transcript levels in various mouse tissues in response to treatment with Compound 1 and NP extract. Mice were gavaged daily with cymopol 1 or NP extract (two different doses each) for 3 days and tissues harvested 12 h after the last treatment. mRNA levels were analyzed by RT-qPCR by TaqMan (endogenous control beta actin) (* indicates p-value <0.05; ** indicates p-value <0.005; *** indicates p-values <0.0005).

FIG. 42. depicts Hmox1 transcript levels in various mouse tissues in response to treatment with Compound 1 and NP extract. Mice were gavaged daily with cymopol 1 or NP extract (two different doses each) for 3 days and tissues harvested 12 h after the last treatment. mRNA levels were analyzed by RT-qPCR by TaqMan (endogenous control beta actin) (* indicates p-value <0.05; ** indicates p-value <0.005; *** indicates p-values <0.0005).

FIG. 43. depicts a dose optimization of NP extract concentration for DSS model. Age-matched WT C57Bl/6 mice (6-8 weeks old) received daily doses of 1.0, 1.5 or 2.0 mg/kg NP extract for 3 days, and cecum and large intestines were harvested 12 h after the last dose. Nqo1 and Hmox1 mRNA levels were analyzed by RT-qPCR by TaqMan (endogenous control beta actin).

FIG. 44. depicts a heatmap representing the mean log₁₀ normalized relative abundances of bacterial families that were significantly different (FDR P<0.05) between control and low groups.

FIG. 45. depicts a heatmap representing the mean log₁₀ normalized relative abundances of bacterial families that were significantly different (FDR P<0.05) between control and high groups.

FIG. 46. depicts a heatmap representing the mean log₁₀ normalized relative abundances of bacterial families that were significantly different (FDR P<0.05) between high and low groups.

FIG. 47. depicts a shift in mouse gut microbiota based on PCoA on QIIME close-reference OTUs generated from the reverse reads between control and low groups, PCoA1 FDR P<0.0001.

FIG. 48. depicts a shift in mouse gut microbiota based on PCoA on QIIME close-reference OTUs generated from the reverse reads between control and high groups, PCoA1 FDR P<0.05.

FIG. 49. depicts a shift in mouse gut microbiota based on PCoA on QIIME close-reference OTUs generated from the reverse reads between high and low groups, PCoA1 FDR P<0.0001.

FIG. 50. depicts a shift in mouse gut microbiota based on PCoA on QIIME close-reference OTUs generated from the centrifuge classified reads between control and low groups PCoA1 FDR P<0.0001.

FIG. 51. depicts a shift in mouse gut microbiota based on PCoA on QIIME close-reference OTUs generated from the centrifuge classified reads between control and high groups, PCoA1 FDR P<0.05.

FIG. 52. depicts a shift in mouse gut microbiota based on PCoA on QIIME close-reference OTUs generated from the centrifuge classified reads between high and low groups, PCoA1 FDR P<0.0001.

FIG. 53. depicts a greater increase in Nqo1 activity in wild-type MEF cells to than in Nrf2−/− and Keap1−/− MEFs by Compound 1 and the NP extract. Asterisks indicate p<0.05 which correlate to the indicated color. Significant values indicated with the black asterisk indicate concentrations which are increased over DMSO control in the same cell line. The green and blue asterisks indicate significance of induction relative to the corresponding knockouts at the same concentration. MEF cells were chemically induced by pre-treating with IFN-γ and TNF-α.

FIG. 54. depicts representative chymotryptic peptide spectrum containing CymQ(+242) alkylated Cys196. The total ion current (TIC) peak area (>95% confidence level) for each CymQ adduct (242, 244, 320 and 322) from all experiments were summed for individual cysteine residues where modification occurred. The most abundant modification across multiple cysteine residues was shown to be with CymQ(+242) adduction.

FIG. 55. depicts summed CymQ modifications for different cysteines of Keap1 protein from three separate experiments using five different sample preparation conditions. The total ion current (TIC) peak area (>95% confidence level) for each CymQ adduct (242, 244, 320 and 322) from all experiments were summed for individual cysteine residues where modification occurred. The most abundant modification across multiple cysteine residues was shown to be with CymQ(+242) adduction.

FIG. 56. depicts the reduction of NO levels by Compound 1 and Cymopolia sp. non-polar extract (NP extract) in an IFN-gamma macrophage model.

FIG. 57. depicts induction of Nqo1 in cecum and large intestines (* indicates p-value <0.05; ** indicates p-value <0.005; *** indicates p-values <0.0005).

FIG. 58. depicts induction of Hmox1 in cecum and large intestines (* indicates p-value <0.05; ** indicates p-value <0.005; *** indicates p-values <0.0005).

FIG. 59. depicts PCA analysis of microbial gene expression showing significant changes in microbial gene expression between control and low groups PC1 FDR P<0.05.

FIG. 60. depicts PCA analysis of microbial gene expression showing significant changes in microbial gene expression between control and high groups, PC1 FDR P<0.01.

FIG. 61. depicts PCA analysis of microbial gene expression showing significant changes in microbial gene expression between high and low groups.

FIG. 62. depicts heatmaps showing the mean log 2 edgeR normalized gene expression of representative significantly differentially (FDR P<0.05) expressed genes between control and low groups.

FIG. 63. depicts heatmaps showing the mean log 2 edgeR normalized gene expression of representative significantly differentially (FDR P<0.05) expressed genes between control and high groups.

FIG. 64. depicts heatmaps showing the mean log 2 edgeR normalized gene expression of representative significantly differentially (FDR P<0.05) expressed genes between high and low groups.

### DETAILED DESCRIPTION

### Definitions

In order that the invention may be more readily understood, certain terms are first defined here for convenience.

As used herein, the term “treating” a disorder encompasses preventing, ameliorating, mitigating and/or managing the disorder and/or conditions that may cause the disorder. The terms “treating” and “treatment” refer to a method of alleviating or abating a disease and/or its attendant symptoms. In accordance with the present invention “treating” includes preventing, blocking, inhibiting, attenuating, protecting against, modulating, reversing the effects of and reducing the occurrence of e.g., the harmful effects of a disorder.

As used herein, “inhibiting” encompasses preventing, reducing and halting progression.

As used herein, “activating” encompasses permitting, increasing and enhancing progression.

As used herein, “enriched” encompasses greater or increased amounts of a material or desired or active compound or agent relative to its natural or other reference state.

As used herein, as “extract” is a preparation of constituents of a material (e.g., seaweed), including for example, solvent extracts, concentrated forms of said constituents, concentrated solvent extracts, isolated chemical compounds or mixtures thereof.

The term “modulate” refers to increases or decreases in the activity of a cell in response to exposure to a compound of the invention.

The terms “cultivated,” “cultivate,” and “cultivation” refer to material that is grown under controlled conditions or the process of growing material under controlled conditions. This material also refers to those obtained or purchased that were grown under controlled conditions.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. Particularly, in embodiments the compound is at least 85% pure, more preferably at least 90% pure, more preferably at least 95% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “peptide” is a sequence of at least two amino acids. Peptides can consist of short as well as long amino acid sequences, including proteins.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

The term “protein” refers to series of amino acid residues connected one to the other by peptide bonds between the alpha-amino and carboxy groups of adjacent residues.

Amino acids may be referred to herein by either their commonly known three letter symbols or by the one-letter symbols recommended by the IUPAC-IUB Biochemical Nomenclature Commission.

As to amino acid sequences, one of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and a-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the elastase inhibitor compound are outweighed by the therapeutically beneficial effects.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The term “therapeutically effective amount” refers to that amount of the compound being administered sufficient to prevent development of or alleviate to some extent one or more of the symptoms of the condition or disorder being treated.

A therapeutically effective amount of compound (i.e., an effective dosage) may range from about 0.005 mg/kg to about 1000 mg/kg, preferably about 0.1 mg/kg to about 1000 mg/kg, more preferably about 10 mg/kg to about 500 mg/kg of body weight. In other embodiments, the therapeutically effective amount may range from about 0.10 nM to about 50004. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. It will also be appreciated that the effective dosage of a compound used for treatment may increase or decrease over the course of a particular treatment.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included. In aspects, the compounds of the invention are prodrugs of any of the formulae herein.

The term “subject” refers to animals such as mammals, including, but not limited to, primates (e.g., humans), cows, sheep, goats, horses, dogs, cats, rabbits, rats, mice and the like. In certain embodiments, the subject is a human.

Furthermore the compounds of the invention include olefins having either geometry: “Z” refers to what is referred to as a “cis” (same side) conformation whereas “E” refers to what is referred to as a “trans” (opposite side) conformation. With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

As used herein, the term “alkyl” refers to a straight-chained or branched hydrocarbon group containing 1 to 12 carbon atoms. The term “lower alkyl” refers to a C1-C6 alkyl chain. Examples of alkyl groups include methyl, ethyl, n-propyl, isopropyl, tert-butyl, and n-pentyl. Alkyl groups may be optionally substituted with one or more substituents.

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The sp² or sp carbons of an alkenyl group and an alkynyl group, respectively, may optionally be the point of attachment of the alkenyl or alkynyl groups.

The term “alkoxy” refers to an —O-alkyl radical.

As used herein, the term “halogen”, “hal” or “halo” means —F, —Cl, —Br or —I.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alkylating agents are any reagent that is capable of effecting the alkylation of the functional group at issue (e.g., oxygen atom of an alcohol, nitrogen atom of an amino group). Alkylating agents are known in the art, including in the references cited herein, and include alkyl halides (e.g., methyl iodide, benzyl bromide or chloride), alkyl sulfates (e.g., methyl sulfate), or other alkyl group-leaving group combinations known in the art. Leaving groups are any stable species that can detach from a molecule during a reaction (e.g., elimination reaction, substitution reaction) and are known in the art, including in the references cited herein, and include halides (e.g., I-, Cl-, Br-, F-), hydroxy, alkoxy (e.g., —OMe, —O-t-Bu), acyloxy anions (e.g., —OAc, —OC(O)CF₃), sulfonates (e.g., mesyl, tosyl), acetamides (e.g., —NHC(O)Me), carbamates (e.g., N(Me)C(O)Ot-Bu), phosphonates (e.g., —OP(O)(OEt)₂), water or alcohols (protic conditions), and the like.

In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy.

### Compounds of the Invention

Compounds (e.g., isolated compounds, compounds within seaweed extracts, compounds fractionated from seaweed extracts) of the invention can be made by means known in the art of organic synthesis. Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database.

The compounds herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds described herein, even though only a single tautomeric form may be represented. All such isomeric forms of such compounds herein are expressly included in the present invention. All crystal forms and polymorphs of the compounds described herein are expressly included in the present invention. Also embodied are extracts and fractions comprising compounds of the invention. The term isomers is intended to include diastereoisomers, enantiomers, regioisomers, structural isomers, rotational isomers, tautomers, and the like. For compounds which contain one or more stereogenic centers, e.g., chiral compounds, the methods of the invention may be carried out with an enantiomerically enriched compound, a racemate, or a mixture of diastereomers.

The present invention also contemplates solvates (e.g., hydrates) of a compound of herein, compositions thereof, and their use in the treatment and/or prevention of reactive oxygen species (ROS)-mediated diseases and diseases alleviated or prevented through the activation of the Nrf2-ARE (antioxidant response element) pathway. As used herein, “solvate” refers to the physical association of a compound of the invention with one or more solvent or water molecules, whether organic or inorganic. In certain instances, the solvate is capable of isolation, for example, when one or more solvate molecules are incorporated in the crystal lattice of the crystalline solid.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of the invention is administered to cells or a subject.

### Methods of Treatment

This invention is directed towards seaweed extract compositions, enriched active fractions, processes for isolation, isolated active agents, and methods of treating and/or preventing diseases and disorders by use of the extracts, compounds, and compositions delineated herein.

In other aspects, the invention provides a method of treating and/or preventing a disease, disorder, or symptom thereof in a subject, comprising administering to the subject any compound, seaweed extract, or enriched seaweed extract herein. In another aspect, the compound, seaweed extract, or enriched seaweed extract is administered in an amount and under conditions sufficient to ameliorate the disease, disorder, or symptom thereof in a subject.

The methods can further comprise that wherein the composition is an extract of Cymopolia sp., an enriched extract, or an isolated compound or compound mixture that occurs in a seaweed extract herein.

In another aspect, the algae comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

Another aspect is where the seaweed extract comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

Another aspect is where the seaweed extract is enriched (by any of the processes known in the art and/or specifically delineated herein) in one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

In another aspect the invention provides one or more isolated compounds that is selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

In other aspects, the invention provides a method of modulating Nrf2-ARE activity in a subject, comprising contacting the subject with any compound, seaweed extract, or enriched seaweed extract herein, in an amount and under conditions sufficient to modulate Nrf2-ARE activity. In another aspect, the modulation is activation.

In other aspects, the invention provides a method of modulating the proliferation activity in a subject, comprising contacting the subject with any compound, seaweed extract, or enriched seaweed extract herein, in an amount and under conditions sufficient to modulate proliferation activity.

In one aspect, the invention provides a method of treating a subject suffering from or susceptible to a proliferation related disorder or disease, comprising administering to the subject an effective amount of a compound, seaweed extract, or enriched seaweed extract or pharmaceutical composition of any compound, seaweed extract, or enriched seaweed extract herein.

In another aspect, the invention provides a method of treating a subject suffering from or susceptible to a ROS-mediated disorder or disease, comprising administering to the subject an effective amount of a compound, seaweed extract, or enriched seaweed extract or pharmaceutical composition of any compound, seaweed extract, or enriched seaweed extract herein.

In another aspect, the invention provides a method of treating a subject suffering from or susceptible to a disorder or disease alleviated or prevented through the Nrf2-ARE pathway, comprising administering to the subject an effective amount of a compound, seaweed extract, or enriched seaweed extract or pharmaceutical composition of any compound, seaweed extract, or enriched seaweed extract herein.

Another aspect is where the seaweed is Cymopolia sp.

In certain embodiments, the invention provides a method as described above, wherein the seaweed and/or seaweed extract comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

Another aspect is where the seaweed extract comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

In certain embodiments, the invention provides a method of treating and/or preventing a disorder, wherein the disorder is proliferative diseases and disorders, inflammation (e.g., Crohn's Disease, irritable bowel syndrome (IBS), inflammatory bowel disease (IBD), rheumatoid arthritis, psoriasis, psoriatic arthritis, or ankylosing spondylitis), cancer (e.g., colon cancer, rectal cancer, stomach cancer, or prostate cancer), Alzheimer's disease and other neurodegenerative disorders, stroke, chronic kidney disease, type II diabetes, and aging itself, and other diseases mediated through ROS or alleviated or prevented through the Nrf2-ARE pathway.

In certain embodiments, the methods are useful in providing and/or enhancing anti-aging properties of skin by preventing (e.g., UVA-induced, UVB-induced, photo-damage, aging) wrinkle formation. In certain embodiments, the methods herein are useful in providing and/or enhancing skin tone and skin appearance properties of skin by administration of a topical formulation of compounds and compositions herein to the skin.

In certain embodiments, the compounds and compositions herein are useful in providing and/or enhancing anti-aging properties of skin by preventing (e.g., UVA-induced, UVB-induced, photo-damage, aging) wrinkle formation. In certain embodiments, the compounds and compositions herein are useful in providing and/or enhancing skin tone and skin appearance properties of skin by administration of a topical formulation to the skin.

In certain embodiments, the subject is a mammal, preferably a primate or human.

In another embodiment, the invention provides a method as described above, wherein the effective amount of the compound, seaweed extract, or enriched seaweed extract ranges from about 0.005 μg/kg to about 500 mg/kg, preferably about 0.1 mg/kg to about 500 mg/kg, more preferably about 10 mg/kg to about 500 mg/kg of body weight.

In other embodiments, the invention provides a method as described above wherein the effective amount of the compound, seaweed extract, or enriched seaweed extract ranges from about 1.0 nM to about 500 μM. In another embodiment, the effective amount ranges from about 100 nM to about 100 μM.

In other embodiments, the invention provides a method as described above wherein the effective amount of the compound, seaweed extract, or enriched seaweed extract ranges from about 0.1 mg/ml to about 1000 mg/ml. In certain embodiments, the effective amount ranges from about 1.0 mg/ml to about 500 mg/ml. In another embodiment, the effective amount ranges from about 1.0 mg/ml to about 100 mg/ml.

In another embodiment, the invention provides a method as described above, wherein the compound, seaweed extract, or enriched seaweed extract is administered intravenously, intramuscularly, subcutaneously, intracerebroventricularly, orally, ocularly, or topically.

In another embodiment, the invention provides a method as described herein wherein the compound, seaweed extract, or enriched seaweed extract demonstrates selectivity (e.g., at least 2-fold, at least 5-fold, at least 10-fold, at least X-fold where X is any number between 1 and 20 inclusive) in cell growth activity (e.g., in transformed/nontransformed, MDA-MB-231/NMuMG, U2OS/NIH3T3 cells). In another aspect, the compound, seaweed extract, or enriched seaweed extract demonstrates selectivity in modulating cell growth activity (e.g., at least 2-fold, at least 5-fold, at least 10-fold, at least X-fold where X is any number between 1 and 20 inclusive) relative to another standard anticancer therapy (e.g., paclitaxel, actinomycin D, doxorubicin).

In other embodiments, the invention provides a method as described above, wherein the compound, seaweed extract, or enriched seaweed extract is administered alone or in combination with one or more other therapeutics. In a further embodiment, the additional therapeutic agent is an anti-cancer agent, chemotherapeutic agent, an anti-angiogenesis agent, cytotoxic agent, or an anti-proliferation agent. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, vincristine, vinblastine, etoposide, trimetrexate, teniposide, cisplatin and diethylstilbestrol (DES). See, generally, The Merck Manual of Diagnosis and Therapy, 15th Ed., pp. 1206-1228, Berkow et al., eds., Rahay, N.J., 1987).

Another object of the present invention is the use of a compound, seaweed extract, or enriched seaweed extract as described herein (e.g., of any formulae herein) in the manufacture of a medicament for use in the treatment and/or prevention of a cell proliferation disorder or disease, or to affect cell differentiation, dedifferentiation or transdifferentiation. Another object of the present invention is the use of a compound, seaweed extract, or enriched seaweed extract as described herein (e.g., of any formulae herein) for use in the treatment and/or prevention of a cell proliferation disorder or disease, or affect cell differentiation, dedifferentiation or transdifferentiation.

Another object of the present invention is the use of a compound, seaweed extract, or enriched seaweed extract as described herein (e.g., of any formulae herein) for use in the treatment and/or prevention of a ROS-mediated disorder or disease, or a disease alleviated or prevented through the Nrf2-ARE pathway. Another object of the present invention is where the disease or disorder includes proliferative diseases and disorders, inflammation (e.g., Crohn's Disease, irritable bowel syndrome (IBS), inflammatory bowel disease (IBD), rheumatoid arthritis, psoriasis, psoriatic arthritis, or ankylosing spondylitis), cancer (e.g., colon cancer, rectal cancer, stomach cancer, or prostate cancer), Alzheimer's disease and other neurodegenerative disorders, stroke, chronic kidney disease, type II diabetes, and aging itself, and other diseases mediated through ROS or alleviated or prevented through the Nrf2-ARE pathway.

In another aspect, the invention provides a method of altering the microbiome in the gastrointestinal tract of a subject, comprising administering to the subject an effective amount of a compound, seaweed extract, or enriched seaweed extract or pharmaceutical composition of any compound, seaweed extract, or enriched seaweed extract herein.

In another aspect, the invention provides a method of improving the overall health of a subject, comprising administering to the subject an effective amount of a compound, seaweed extract, or enriched seaweed extract or pharmaceutical composition of any compound, seaweed extract, or enriched seaweed extract herein.

In another aspect, the invention provides a method of supplementing diet in a subject, comprising administering to the subject an effective amount of a compound, seaweed extract, or enriched seaweed extract or pharmaceutical composition of any compound, seaweed extract, or enriched seaweed extract herein.

In another aspect, the invention provides a method of manufacturing a dietary supplement comprising combining a compound, seaweed extract, or enriched seaweed extract or pharmaceutical composition of any compound, seaweed extract, or enriched seaweed extract herein with a carrier. In another aspect, the carrier is suitable for oral administration.

### Pharmaceutical Compositions

In one aspect, the invention provides a pharmaceutical composition comprising the compound, seaweed extract, or enriched seaweed extract and a pharmaceutically acceptable carrier.

In one embodiment, the invention provides a pharmaceutical composition wherein the seaweed, compound, seaweed extract, or enriched seaweed extract comprises and/or is enriched in one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4);

and a pharmaceutically acceptable carrier.

In another embodiment, the invention provides a pharmaceutical composition wherein the compound, seaweed extract, or enriched seaweed extract is an extract from the alga Cymopolia sp., and a pharmaceutically acceptable carrier.

In another embodiment, the invention provides a pharmaceutical composition further comprising an additional therapeutic agent. In a further embodiment, the additional therapeutic agent is an anti-cancer agent, chemotherapeutic agent, an anti-angiogenesis agent, cytotoxic agent, or an anti-proliferation agent.

In one aspect, the invention provides a kit comprising an effective amount of a the compound, seaweed extract, or enriched seaweed extract, in unit dosage form, together with instructions for administering the compound to a subject suffering from or susceptible to a ROS mediated disease or disorder, including proliferative diseases and disorders, inflammation (e.g., Crohn's Disease, irritable bowel syndrome (IBS), inflammatory bowel disease (IBD), rheumatoid arthritis, psoriasis, psoriatic arthritis, or ankylosing spondylitis), cancer (e.g., colon cancer, rectal cancer, stomach cancer, or prostate cancer), stroke, chronic kidney disease, type II diabetes, and aging itself, and other diseases mediated through ROS or alleviated or prevented through the Nrf2-ARE pathway, Alzheimer's disease and other neurodegenerative disorders, memory loss, inducing neurogenesis, enhancing memory retention, enhancing memory formation, increasing synaptic potential or transmission, or increasing long term potentiation (LTP), etc.

In one aspect, the invention provides a kit comprising an effective amount of a compound, seaweed extract, or enriched seaweed extract, in unit dosage form, together with instructions for administering the compound to a subject suffering from or susceptible to a cell proliferation disease or disorder, including cancer (e.g., colon cancer, rectal cancer, stomach cancer, or prostate cancer), solid tumor, angiogenesis, etc.

In one aspect, the invention provides a kit comprising an effective amount of a compound, seaweed extract, or enriched seaweed extract, in unit dosage form, together with instructions for administering the compound to a subject suffering from or susceptible to a disease or disorder alleviated or prevented through the Nrf2-ARE pathway, including proliferative diseases and disorders, inflammation (e.g., Crohn's Disease, irritable bowel syndrome (IBS), inflammatory bowel disease (IBD), rheumatoid arthritis, psoriasis, psoriatic arthritis, or ankylosing spondylitis), cancer (e.g., colon cancer, rectal cancer, stomach cancer, or prostate cancer), stroke, chronic kidney disease, type II diabetes, and aging itself, and other diseases mediated through ROS or alleviated or prevented through the Nrf2-ARE pathway, Alzheimer's disease and other neurodegenerative disorders, etc.

In another aspect, the invention provides a kit comprising an effective amount of a compound, seaweed extract, or enriched seaweed extract or pharmaceutical composition of any compound, seaweed extract, or enriched seaweed extract herein, together with instructions for administering the compound to a subject for altering the microbiome in the gastrointestinal tract of said subject.

In another aspect, the invention provides a kit comprising an effective amount of a compound, seaweed extract, or enriched seaweed extract or pharmaceutical composition of any compound, seaweed extract, or enriched seaweed extract herein, together with instructions for administering the compound to a subject for improving the overall health of said subject.

In another aspect, the invention provides a kit comprising an effective amount of a compound, seaweed extract, or enriched seaweed extract or pharmaceutical composition of any compound, seaweed extract, or enriched seaweed extract herein, together with instructions for administering the compound to a subject supplementing diet of said subject.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The invention also provides a pharmaceutical composition, comprising an effective amount a compound described herein and a pharmaceutically acceptable carrier. In an embodiment, compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic (or unacceptably toxic) to the patient.

In use, at least one compound according to the present invention is administered in a pharmaceutically effective amount to a subject in need thereof in a pharmaceutical carrier by intravenous, intramuscular, subcutaneous, or intracerebro ventricular injection or by oral administration or topical application. In accordance with the present invention, a compound of the invention may be administered alone or in conjunction with a second, different therapeutic. By “in conjunction with” is meant together, substantially simultaneously or sequentially. In one embodiment, a compound of the invention is administered acutely. The compound of the invention may therefore be administered for a short course of treatment, such as for about 1 day to about 1 week. In another embodiment, the compound of the invention may be administered over a longer period of time to ameliorate chronic disorders, such as, for example, for about one week to several months depending upon the condition to be treated.

By “pharmaceutically effective amount” as used herein is meant an amount of a compound of the invention, high enough to significantly positively modify the condition to be treated but low enough to avoid serious side effects (at a reasonable benefit/risk ratio), within the scope of sound medical judgment. A pharmaceutically effective amount of a compound of the invention will vary with the particular goal to be achieved, the age and physical condition of the patient being treated, the severity of the underlying disease, the duration of treatment, the nature of concurrent therapy and the specific organozinc compound employed. For example, a therapeutically effective amount of a compound of the invention administered to a child or a neonate will be reduced proportionately in accordance with sound medical judgment. The effective amount of a compound of the invention will thus be the minimum amount which will provide the desired effect.

A decided practical advantage of the present invention is that the compound may be administered in a convenient manner such as by intravenous, intramuscular, subcutaneous, oral, ocularly, or intra-cerebroventricular injection routes or by topical application, such as in creams or gels. Depending on the route of administration, the active ingredients which comprise a compound of the invention may be required to be coated in a material to protect the compound from the action of enzymes, acids and other natural conditions which may inactivate the compound. In order to administer a compound of the invention by other than parenteral administration, the compound can be coated by, or administered with, a material to prevent inactivation.

The compound may be administered parenterally or intraperitoneally. Dispersions can also be prepared, for example, in glycerol, liquid polyethylene glycols, and mixtures thereof, and in oils.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage. The carrier can be a solvent or dispersion medium containing, for example, water, DMSO, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the compound of the invention in the required amount in the appropriate solvent with various of the other ingredients enumerated above, as required, followed by filtered sterilization. Generally, dispersions are prepared by incorporating the various sterilized compounds into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yields a powder of the active ingredient plus any additional desired ingredient from previously sterile-filtered solution thereof.

For oral therapeutic administration, the compound may be incorporated with excipients and used in the form of ingestible tablets, buccal tablets, troches, capsules, elixirs, suspensions, syrups, wafers, and the like. Compositions or preparations according to the present invention are prepared so that an oral dosage unit form contains compound concentration sufficient to treat a disorder in a subject.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present.

Topical administration of the pharmaceutical compositions of this invention is especially useful when the desired treatment involves areas or organs readily accessible by topical application. For topical application topically to the skin, the pharmaceutical composition should be formulated with a suitable ointment, lotion, or cream containing the active components suspended or dissolved in a carrier. Carriers for topical administration of the compounds of this invention include, but are not limited to, water, phenoxyethanol, citric acid, phosphoric acid, succinic acid, steareth-20, potassium sorbate, methylparaben, propylparaben, butylparaben, ethylparaben, isobutylparaben, glyceryl stearate, dimethicone, capryl glycol, triethanolamine, maltodextrin, sorbic acid, ethylene brassylate, methyl linalool, isobutyl methyl tetrahydropyranol, phenonip, tocopheryl acetate, prodew 400, isododecane, pentylene glycol, capric/caprylic triglyceride, shea butter, cetyl alcohol, stearic acid, polysorbate 80, xanthan gum, C₁₂-C₁₅ alkyl benzoate, sunscreen agents, sodium cocoamphodiacetate, sodium methyl cocoyl taurate, lactose, talc, silicic acid, aluminum hydroxide, calcium silicates, polyamide powder, animal and vegetable fats, oils, waxes, paraffins (e.g., liquid paraffin, isoparaffin, soft paraffin), starch, tragacanth, cellulose derivatives, polyethylene glycols (e.g., polyethylene glycol, PEG-100 stearate, hexadecyl stearate, decyl stearate, isopropyl isostearate, stearyl stearate; aluminium stearate, glyceryl monostearate, PEG-12 dimethicone, polyethylene glycol (200-6000) mono- and di-fatty acid esters, PEG-300 or PEG-400), silicones, zinc oxide, propylene glycol, dipropylene glycol, polypropylene glycol, sorbitol, hydroxypropyl sorbitol, hexylene glycol, 1,3-butylene glycol, 1,3-butylene glycol monostearate, 1,3-butylene glycol distearate, 1,2,6-hexanetriol, ethoxylated glycerin, propoxylated glycerin, ethylhexylglycerin, xylitol, hexyl laurate, isohexyl laurate, isohexyl palmitate, ethylhexyl palmitate, isopropyl palmirate, decyl oleate, isodecyl oleate, diisopropyl adipate, diisohexyl adipate, dihexyldecyl adipate, diisopropyl sebacate, lauryl lactate, myristyl lactate, cetyl lactate, oleyl myristate, myristyl myristate, oleyl stearate, oleyl oleate; ethylene glycol mono- and di-fatty acid esters, diethylene glycol mono- and di-fatty acid esters, polyglycerol poly-fatty esters, ethoxylated glyceryl monostearate, polyoxyethylene polyol fatty acid ester, sorbitan fatty acid esters (e.g., sorbitan isostearate, polyoxyethylene sorbitan fatty acid esters), cellulose polymers, carbomer polymers, carbomer derivatives, essential oils, terpenes, oxazoldines, surfactants, polyols, azone and azone derivatives, microcrystalline wax, wax esters such as beeswax, spermaceti, terol esters, cholesterol fatty acid esters, mineral oil, polyalphaolefins, petrolatum, polybutenes, lays (e.g., Montmorillonite, Hectorite, Laponite Bentonite), mica, silica, alumina, zeolites, sodium sulfate, sodium bicarbonate, sodium carbonate, calcium sulfate, fatty acid soaps, sodium lauryl sulfate, sodium lauryl ether sulfate, alkyl benzene sulfonate, mono- and di-alkyl acid phosphates, sarcosinates, taurates, sodium fatty acyl isethionate; dialkylamine oxide, betaines (e.g., betaine, cocamidopropyl betaine), vegetable oil (e.g., arachis oil, castor oil and the like), cetostearyl alcohol, wool-fat, non-ionic emulsifying agents, glycerol, cottonseed oil, groundnut oil, olive oil, sesame oil, soybean oil, cresols, benzyl alcohol, phenyllic alcohol, mannitol, sucrose, trehalose, glucose, raffinose, arginine, glycine, histidine, dextran, ethylene glycol, ethanol, and methanol. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Ocular administration of the pharmaceutical compositions of this invention is especially useful when the desired treatment involves areas or organs readily accessible by ocular application. For ocular application topically to the eyes, the pharmaceutical composition should be formulated with a suitable liquid, ointment, or cream containing the active components suspended or dissolved in a carrier. Carriers for ocular administration of the compounds of this invention include, but are not limited to, the aforementioned carriers for topical administration in addition to 1,2-dioleoyl-3-trimethylammonium-propane chloride (DOTAP), 1,2-distearoyl-SN-glycero-3-phosphocholine, alpha-tocopherol polyethylene glycol succinate, arginine octadecylamine, castor oil, chitosan, dextrose, gellan gum, hydroxypropylmethyl cellulose (HPMC), lecithins (egg and soybean), mannitol, oleylamine, poly(D, L-lactide-co-glycolide acid) (PLGA), Poloxamer 188, Poloxamer 407, Poloxamer CRL 1005, poly (ε-caprolactone), poly (N-isopropylacrylamide (PNIPAAm), polyamidoamine (PAMAM), polyethylene glycol 200, polyethylene glycol 40 stearate, poly-hexyl-2-cyanoacrylate, poly-L-lysine (PLL), polymethacrylic acid, polysorbate 80, polyvinyl alcohol, propylene glycol, quaternary ammoniums, sodium alginate, sorbitol, stearylamine, tyloxapol, and water.

For topical administration, the active compound(s), extracts, enriched extracts, or prodrug(s) can be formulated as solutions, gels, lotions, ointments, creams, suspensions, and the like.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

The present invention will now be demonstrated using specific examples that are not to be construed as limiting.

### General Experimental Procedures

HPLC grade solvents were from Fisher Scientific and all other chemicals were purchased from Sigma, unless indicated otherwise. Anti-NQO1 (mouse) and anti-Nrf2 (rabbit) antibodies were purchased from Abcam, anti-Oct-1 (C-21), anti-Gal4 (DBD), and anti-Keap1 (E-20) from Santa Cruz Biotechnology, anti-HA (mouse) from Covance, anti-goat-HRP from Chemicon (Millipore) and anti-β-actin (rabbit), anti-a-tubulin (rabbit), anti-mouse-HRP, and anti-rabbit-HRP from Cell Signaling Technology. Protein A/G-agarose beads were purchased from Santa Cruz Biotechnology and chitin magnetic beads from New England Biolabs.

The ARE-luciferase reporter construct (ARE-luc) contains the core sequence of human NQO1-ARE1. The mock plasmid (pcDNA3-mRFP) was purchased from Addgene (plasmid 13032). The following plasmids were reported by Zhang et a/.2003 [Zhang, D. D., and Hannink, M. (2003) Distinct cysteine residues in Keap1 are required for Keap1-dependent ubiquitination of Nrf2 and for stabilization of Nrf2 by chemopreventive agents and oxidative stress. Mol. Cell. Biol. 23, 8137-8151]. The Keap1-CBD plasmid carries the entire open reading frame (ORF) of human Keap1 fused to the chitin binding domain (CBD) of the Bacillus circulans chitinase A1 gene upstream of the Keap1 stop codon. Oligonucleotide-directed mutagenesis was used to generate the Keap1-C151S-CBD and Keap1-C288S-CBD plasmids. The HA-Cul3 plasmid was constructed by fusing an N-terminal hemagglutinin (HA) tag with a cDNA sequence coding for amino acids 1-380 of human Cul3 gene. The Gal4-Neh2 expression vector contains the codons for the first 97 amino acids (Neh2 domain) of human Nrf2 fused to the ORF of the Gal4 DNA-binding domain

### Example 1: Extraction and Isolation of Enriched Extracts and Compounds 1, 2, 3, and 4 from Cultivated Cymopolia barbata

The Cymopolia barbata was collected in Boca Grande Key Florida in May 2009 and was immediately placed into a −20° C. freezer. The algae was later lyophilized and stored at −20° C. before being extracted with solvents of varying polarities. The non-polar (NP) extract was generated with 1 L EtOAc added to 214.10 g of freeze-dried Cymopolia barbata on a stir plate at room temperature for two consecutive nights, filtering and adding fresh solvent the second day (2 L total). The solvent was dried on a low pressure RotaVapor system and later transferred to small vials and dried under nitrogen gas, yielding 4.09 g of the non-polar (NP) extract. 2 g of the NP extract was further partitioned between hexane and 80% MeOH:H₂O v/v. The 80% solution was adjusted to 50% MeOH:H₂O v/v and partitioned between equal amounts of DCM. 800 mg of the dichloromethane (DCM) fraction (921.66 mg total) was processed through size exclusion chromatography (Sephadex LH20, 40 cm in length×2 cm in diameter) in 50% DCM:MeOH v/v. Nine fractions were collected. The 6^{th} fraction, a yellow band, was collected between 42-50 mL elution (597.53 mg). The fraction was subjected to a C18 column (20 cm height×3 cm diameter) with 2 column volumes of 10% MeOH:H₂O v/v, 2 column volumes of 50% MeOH:H₂O v/v, 4 column volumes of MeOH, and 4 column volumes of iPrOH. The MeOH fraction (557.03 mg) was purified via HPLC using reversed-phase (C18) silica-gel-based SPE columns (Luna C18) in a 70-100% MeOH:H₂O gradient over 32 minutes (2 mL/min). Four major compounds were eluted at 90% (7-hydroxycymopol, 2, 12.11 mg, 0.006%), 93% (cymobarbatol, 3, 100.64 mg, 0.05%), 94% (cymopol, 1, 170.09 mg, 0.09%), and 98% MeOH:H₂O (cyclocymopol monomethyl ether, 4, 38.04 mg, 0.019%).

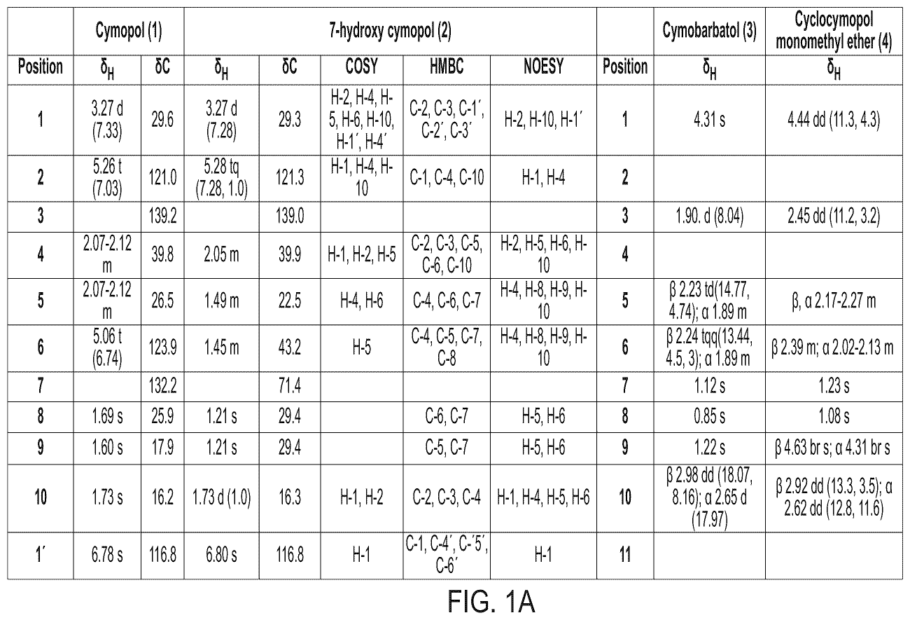

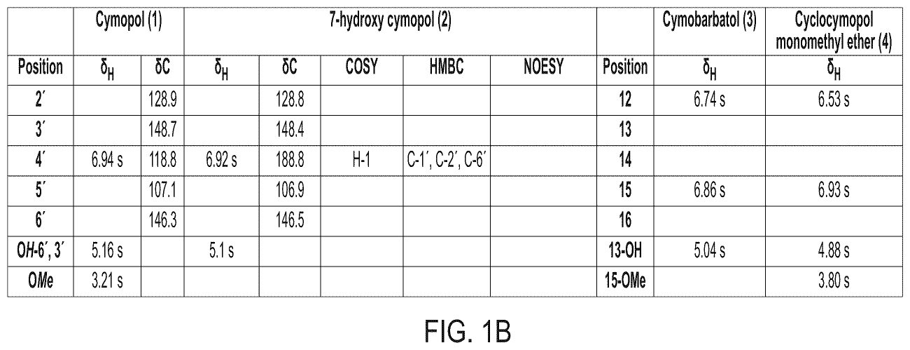

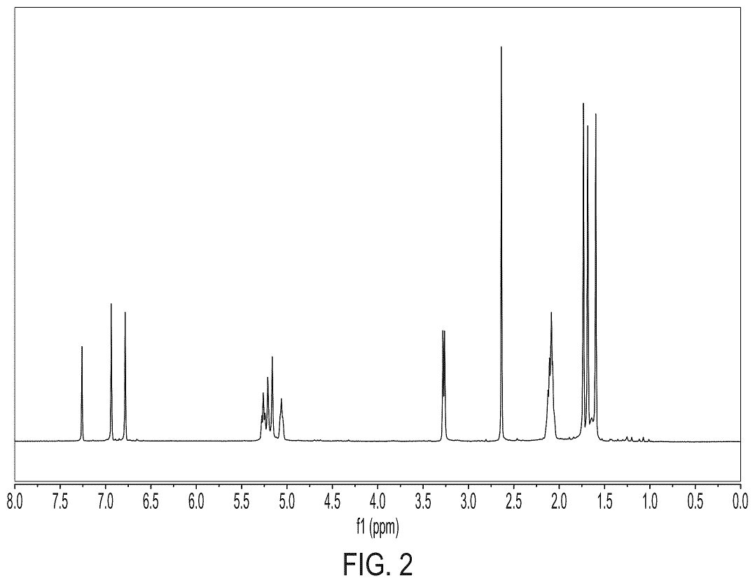

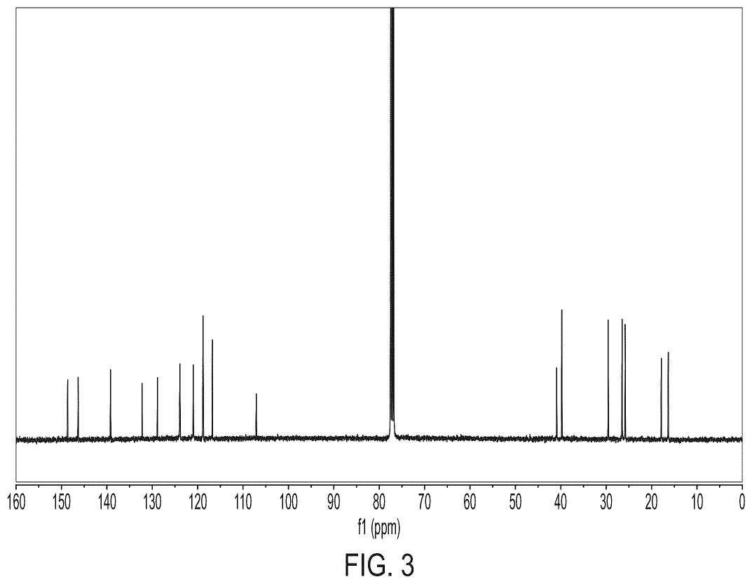

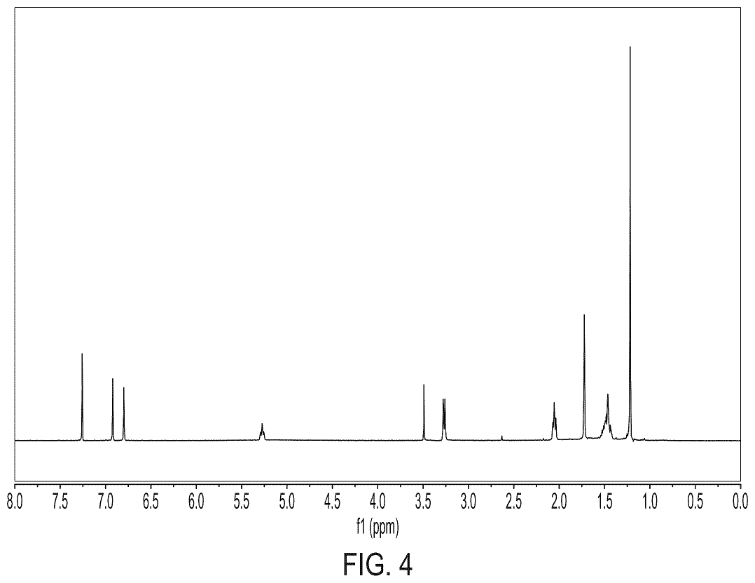

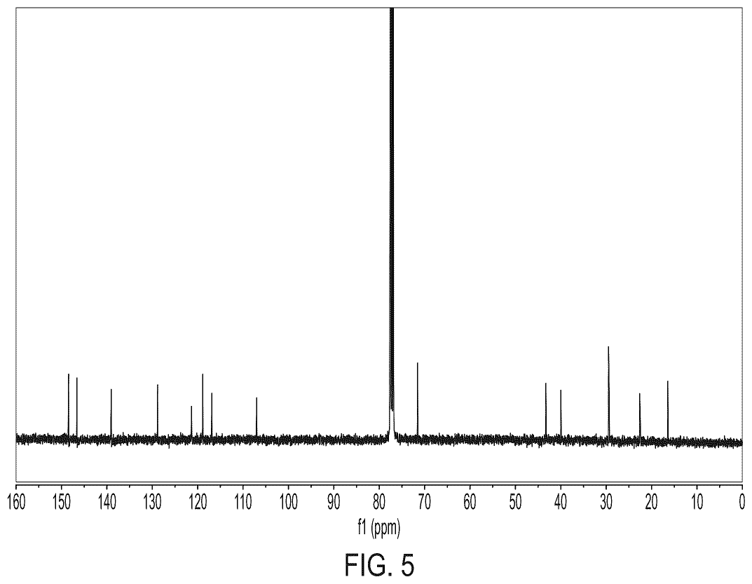

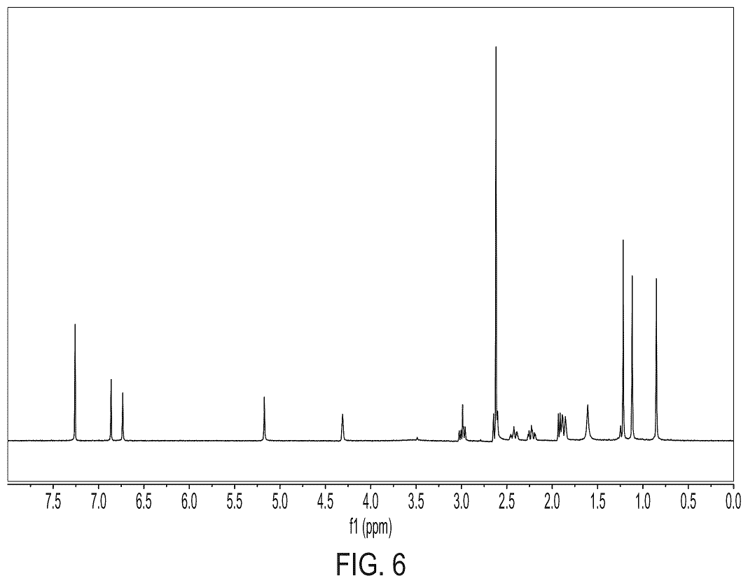

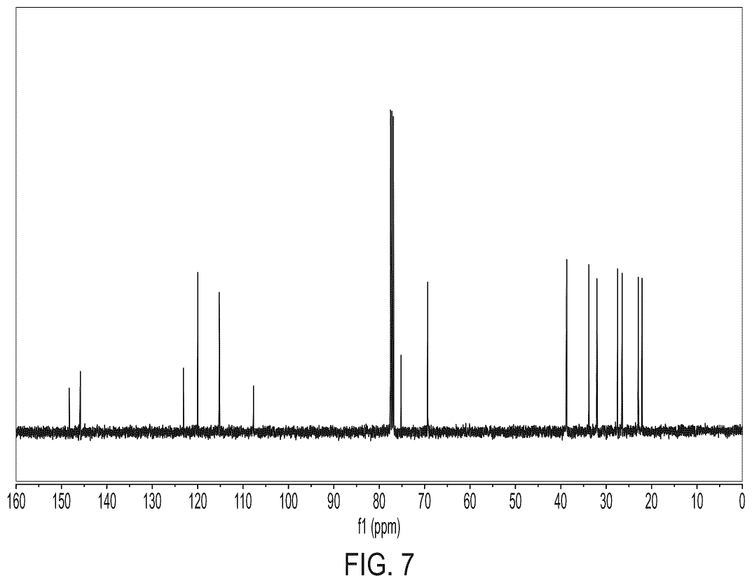

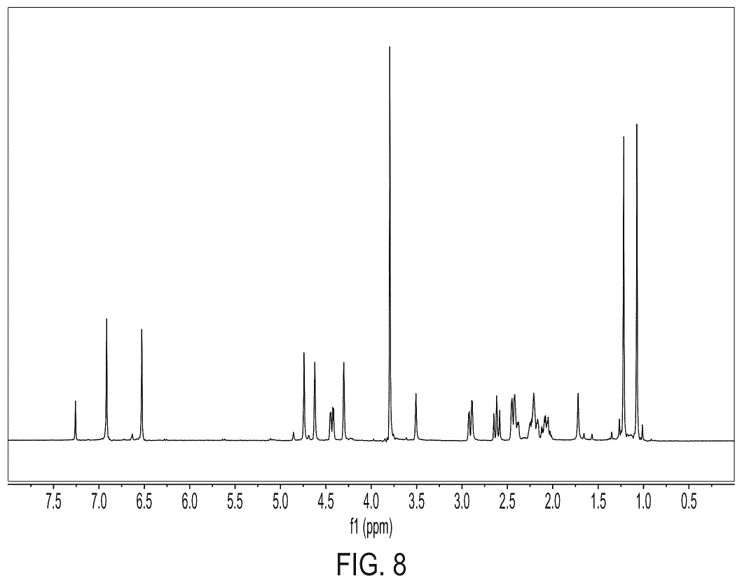

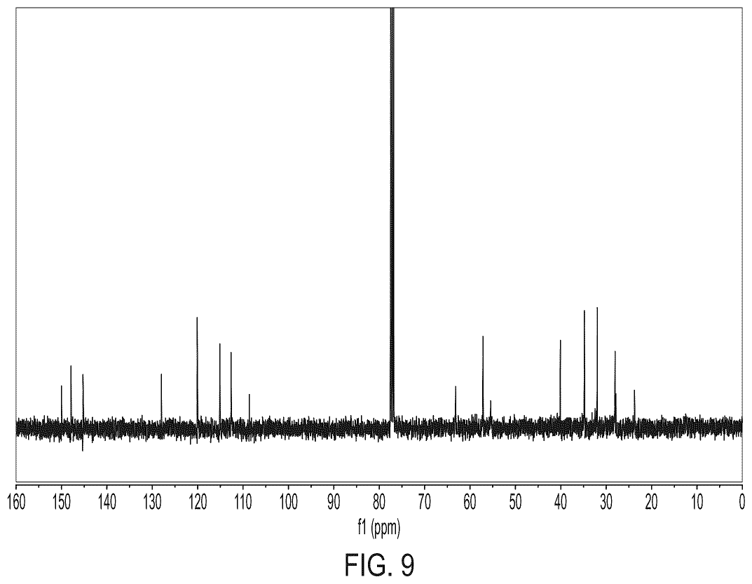

Structures of four isolated compounds were determined using 400 MHz or 600 MHz NMR (1D), 500 MHz NMR (2D) and high resolution electrospray ionization mass spectrometry (HRESIMS) (see, FIGS. 1-9 and Compounds 1-4 below). The compounds were identified as cymopol (1), 7-hydroxycymopol (2), cymobarbatol (3), and cyclocymopol monomethyl ether (4).

### Compound 1: (E)-2-bromo-5-(3,7-dimethylocta-2,6-dien-1-yl)benzene-1,4-diol (Cymopol, 1)

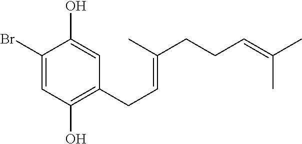 [SMILES: CC(C)=CCC/C(C)=C/Cc1cc(O)c(Br)cc1O]

Cymopol 1 (1): 170.09 mg; ¹H NMR (400 MHz, CDCl₃): 1.60 (3H, s), 1.69 (3H, s), 1.73 (3H, s), 5.0-5.2 (1H, m) 2.04-2.20 (2H, m), 2.04-2.20 (2H, m), 2.28 (2H, d, JJ=7.4), 5.27 (1H, t, J=7.4), 6.79 (1H, S), 6.92 (1H, s). ¹³C NMR (400 MHz, CDCl₃): 29.6, 121.0, 139.2, 39.8, 26.5, 123.9, 132.2, 25.9, 17.9, 16.2, 116.8, 128.9, 48.7, 118.8, 107.1 HRESIMS m/z 322.9/325.2 [M−H]⁻.

### Compound 2: (E)-2-bromo-5-(7-hydroxy-3,7-dimethyloct-2-en-1-yl)benzene-1,4-diol (7-hydroxycymopol, 2)

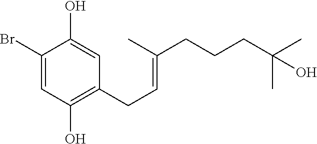 [SMILES: C/C(=C\Cc1cc(O)c(Br)cc1O)CCCC(C)(C)O]

7-hydroxycymopol (2): 12.11 mg: ¹H NMR (400 MHz, CDCl₃): 1.21 (6H, s), 1.45 (2H, m), 1.49 (2H, m), 1.73 (3H s), 2.05 (2H m), 3.27 (2H, d, J=7.28), 5.28 (1H, tq, J=7.28, 1.0), 6.81 (1H, s), 6.92 (1H, s). ¹³C NMR (400 MHz, CDCl₃) 29.3, 121.3, 139.0, 39.9, 22.5, 43.2, 71.4, 29.4, 29.4, 16.3, 116.8, 128.8, 148.4, 188.8, 106.9, 146.5. HRESIMS m/z 341.1/343.0 [M−H]⁻.

### Compound 3: 2,6-dibromo-1,1,4a-trimethyl-2,3,4,4a,9,9a-hexahydro-1H-xanthen-7-ol (Cymobarbatol, 3)

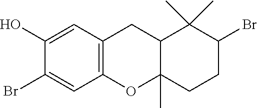 [SMILES: CC12CCC(Br)C(C)(C)C1Cc1cc(O)c(Br)cc1O2]

Cymobarbatol (3): 100.64 mg; ¹H NMR (600 MHz, CDCl₃): 0.85 (s), 1.12 (s), 1.22 (s), 4.31(s), 1.90 (d. J=8.04), 1.89 (α,m), 2.23 (α, td, J=14.77, 4.74), 2.23(β, m), 2.43 (β, dq, J=13.87, 2.83), 2.65, (α, d, J=17.97), 2.98 (β, dd, J=18.07, 8.16), 5.04 (s), 6.74 (s), 6.86 (s). HRESIMS m/z=401/402.9/405 [M−H]⁻.

### Compound 4: 5-bromo-2-((3-bromo-2,2-dimethyl-6-methylenecyclohexyl)methyl)-4-methoxyphenol (Cyclocymopol monomethyl ether, 4)

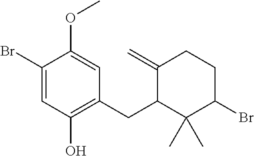 [SMILES: C=C1CCC(Br)C(C)(C)C1Cc1cc(OC)c(Br)cc1O]

Cyclocymopol monomethyl ether (4): 38.04 mg: ¹H NMR (400 MHz, CDCl₃) 12.11 mg, (400 MHz, CDCl₃): 1.08 (3H, s), 1.23 (3H, s), 2.02-2.13 (1H, m), 2.17-2.27 (2H, m), 2.39 (IH, m), 2.45 (1H, dd, J=3.5, 11.2 Hz). 2.62 (IH, dd, J=11.6. 12.8 Hz), 2.92 (IH, dd, J=3.5, 13.3 HZ), 3.80 (3H, s), 4.31 (1H. br s), 4.44 (1H, dd, J=4.4, 11.2 Hz), 4.63 (1H, s, OH), 4.63 (1H. brs), 6.53 (1H, s), 6.93 (1H, s); ¹³C NMR (400 MHz, CDCl₃) δ 27.6, 27.8, 31.8, 34.6, 39.9, 55.1, 57, 63.0, 108.4, 112.3, 115.0, 119.8, 127.7, 144.9, 147.6, 149.6. HRESIMS m/z: 416/418/420 (1:3:1) [M]⁺.

### Example 2: Cell Culture

All cells were maintained at 37° C. in an humidified CO₂ atmosphere in respective media (IMR-32: EMEM; LNCaP: RPMI 1640; RAW264.7: DMEM) supplemented with 10% heat-inactivated FBS from HyClone (Logan, UT).

### Example 3: ARE-luc Reporter Assay in IMR-32 and LNCaP Cells

An ARE-luciferase reporter plasmid and CMV-GFP plasmid (to monitor transfection efficiency) were co-transfected into a human androgen-sensitive prostate cancer cell line, LNCaP (6×10⁴ cells/well), and a human neuroblastoma cell line, IMR-32 (3×10⁴ cells/well), using Fugene® HD (Roche Diagnostics) in 96-well format. LNCaP and IMR-32 cells have been previously used as cellular models of oxidative stress [Li J, Lee J M, Johnson J A. Microarray analysis reveals an antioxidant responsive element-driven gene set involved in conferring protection from an oxidative stress-induced apoptosis in IMR-32 cells. J Biol Chem. 2002; 277:388-394; Lee J M, Hanson J M, Chu W A, Johnson J A. Phosphatidylinositol 3-kinase, not extracellular signal-regulated kinase, regulates activation of the antioxidant-responsive element in IMR-32 human neuroblastoma cells. J Biol Chem. 2001; 276(23):20011-20016; Brooks J D, Paton V G, Vidanes G. Potent induction of phase 2 enzymes in human prostate cells by sulforaphane. Cancer Epidemiol Biomarkers. 2001; 10:949-954; Ratnayake R, Liu Y, Paul V J, Luesch H. Cultivated sea lettuce is a multiorgan protector from oxidative and inflammatory stress by enhancing the endogenous antioxidant defense system. Cancer Prev Res 2013; 6:989-999]. After 24 hours of incubation, cells were treated with extracts from Cymopolia barbata, a negative control (DMSO, 1%, v/v), and a positive control (1011M sulforaphane, SF, and tert-butylhydroquinone, tBHQ, respectively.) 24 hours post-treatment, luciferase activity was detected using BriteLite detection reagent (PerkinElmer). ARE-luc activity was used to guide fractionation of the extracts.

### Example 4: Immunoblot Analysis and Results

IMR-32 cells were seeded into 6-well plates (7×10⁵ cells/well) and incubated at 37° C. for 24 h. The cells were then treated with a solvent control (DMSO, 1%, v/v), isolated compounds, or extracts. After 24 h, cells were lysed in 200 μL of PhosphoSafe buffer (Novagen). Protein concentrations were measured by using a BCA assay kit (Pierce). Equal amounts of total protein were separated using SDS-PAGE (NuPAGE® Novex® 4-12% Bis-Tris Mini gels, Invitrogen) and transferred onto a PVDF membrane. Membranes were blocked overnight with 5% BSA at 4° C. and incubated with indicated primary antibodies for 2 h at room temperature. After washing, the membranes were then incubated with the corresponding secondary antibodies (HRP-linked) for 1 h at room temperature and detected with Supersignal Femto Western Blotting kit (Pierce). For assays in the presence of N-acetylcysteine (NAC), the cells were pretreated with the antioxidant (NAC at 1 mM) for 2 h prior to treatment with the solvent control, isolated compounds, or extracts.

### Example 5: RNA Extraction and Quantitative PCR (qPCR) Analysis for Nrf2 and NQ01 Expression in IMR-32 Cells

The cells were seeded in 6-well format (see immunoblot) and treated with a solvent control (DMSO, 1%, v/v), isolated compounds, or extracts for 12 h. The RNeasy Mini Kit (Qiagen) was used to collect total RNA from cells, while for mouse tissue, TRIzol reagent (Invitrogen) was used. In each case, total RNAs (2 μg) were reverse-transcribed into cDNAs, which were used as templates for TaqMan gene expression assay (Applied Biosystems) and detected using the 7300 Real-Time PCR System (Applied Biosystems). Each qPCR sample was tested in triplicate as a 25 μL total reaction volume (12.5 μL of Taqman 2× universal master mix, 1.25 μL of a 20× TaqMan gene expression assay probe, 1 μL of the above cDNA, and 10.25 μL of RNase-free sterile water). The qPCR method was designed as following: 50° C. for 2 min, 95° C. for 10 min, and 40 cycles of 95° C. for 15 s and 60° C. for 1 min. Endogenous controls used included GAPDH for IMR-32 cells and β-actin to normalize for RAW264.7 cells and mouse tissues.

### Example 6: Separation and Preparation of Cytosolic and Nuclear Extracts

IMR-32 cells were seeded in 6-well format at 2×10⁶ cells/well and incubated at 37° C. overnight. The cells were treated with a solvent control (DMSO, 1%, v/v) or serial dilutions of isolated compounds or extracts for the indicated periods of time. The cytosolic and nuclear extracts were separated and prepared using a commercial kit, NE-PER Nuclear and Cytoplasmic Extraction Reagents (Thermo Scientific). Briefly, cells were gently washed once with 200 μL at DPBS before being resuspended in 1 mL DPBS, transferred to a pre-chilled tube, and pelleted at 500×g for 3 min at 4° C. The supernatant was removed and cells were resuspended in ice-cold CER I containing 1% cOmplete, EDTA-free Protease Inhibitor Cocktail (v/v, Roche Diagnostics) by vigorous vortexing. After addition of ice-cold buffer CER II, the samples were vortexed vigorously for 5 s and incubated on ice for 1 min twice before being pelleted at 16,000×g for 5 min at 4° C. The supernatant was transferred to a fresh tube and used as cytosolic stock. The pellet was washed twice by adding 75 μL ice-cold DPBS, flicking, centrifuging, and removing the supernatant before resuspension in ice-cold buffer NER supplemented with 1% cOmplete, EDTA-free Protease Inhibitor Cocktail (v/v). Samples underwent rounds of vigorous vortexing (15 s) followed by incubation on ice for 10 min over a period of 40 min. Samples were centrifuged at 16,000×g for 10 min at 4° C. The supernatant was used as nuclear extract stocks. Subsequently, the cellular and nuclear extracts were analyzed by immunoblot.

### Example 7: RNA Interference Experiments

IMR-32 cells were seeded into 6-well plates (6×10⁵ cells/well) and incubated for 24 h at 37° C. The media was then carefully aspirated and replaced with a transfection mixture composed of siRNAs (50 nM) and siLentFect™ lipid transfection reagent (Bio-Rad Laboratories) in fresh medium. After another 48 h of incubation, the cells were treated with a solvent control (DMSO, 1%, v/v) or serial dilutions of isolated compounds or extracts for an additional 24 h. Protein was isolated using Phosphosafe (see immunoblot). The siRNAs, siGENOME Non-Targeting siRNA Pools and siGENOME SMARTpool (human NFE2L2), were purchased from Dharmacon.

### Example 8: Glutathione Assays

IMR-32 cells were seeded in 6-well format (8×10⁵ cells/well) and incubated at 37° C. overnight. The glutathione assay was performed using the standard manufacturer's protocol (Sigma). Briefly, the cells were treated with a solvent control (DMSO, 1%, v/v) or serial dilutions of isolated compounds or extracts for the indicated periods of time. Following treatment, cells were very gently washed twice with 100 uL Dulbecco's phosphate buffered saline (DPBS). The cells were then resuspended in 200 μL DPBS and pelleted at 600×g for 10 min at 4° C. The supernatant was aspirated and the pellet was deproteinized and resuspended in 3 volumes (approximately 30 μL) of 5% sulfosalicylic acid solution (v/v). Two freeze-thaw cycles were performed between liquid N₂ and a water bath at 37° C. The suspension was then incubated for 5 min at 4° C. and the cellular debris was pelleted at 10,000×g for 10 min at 4° C. The supernatant was transferred to a new tube and used as the glutathione stock. A kinetic assay was performed measuring the absorbance of 5-thio-2-nitrobenzoic acid (TNB) spectrophotometrically at 412 nm over 10 minutes. A standard curve of reduced GSH was used to determine the amount of GSH in the biological samples. All calculations were performed according to the manufacturer's protocol.

### Example 9: In Vivo Studies

The experiments were approved by the Institutional Animal Care & Use Committee at the University of Florida. Groups of 5-10 wild type male mice (C57BL/6J) were used for each treatment type. The mice were maintained under approved standard conditions. Cymopol (1) and the non-polar extract (NP extract) were dissolved in Chremophor EL (Sigma) containing 10% DMSO and administered via oral gavage for 3 consecutive days, with 24 h between each treatment. No apparent toxicity was observed. 12 h following the final treatment, the mice were euthanized in 100% CO₂. The tissues were harvested immediately, frozen on dry ice, and kept at −80° C. until analyzed.

### Example 10: Assay for Induction of iNOS and Cox2 in Macrophage Cells

RAW264.7 cells were seeded in 6-well plates (8×10⁵ cells/well) and incubated at 37° C. overnight. Cells were treated with a solvent control (DMSO, 1%, v/v) or serial dilutions of isolated compounds or extracts for 1 h before the addition of 1 μg/mL lipopolysaccharide (LPS) and incubated at 37° C. for 12 h before extraction of total RNA (see RNA extraction, cDNA synthesis, and quantitative PCR). qPCR analyses used to detect transcript levels of iNOS, COX2, Nqo1, and β-actin (endogenous control).

### Example 11: NO Assay

RAW264.7 cells (2×10⁴ cells/well), wild-type (8×10³ cells/well), Nrf2−/−(7×10³ cells/well), and Keap1−/−(5×10³ cells/well) MEF were seeded into 96-well format. Cells were pretreated for 1 h with a solvent control (DMSO, 1%, v/v) or serial dilutions of isolated compounds or extracts before addition of LPS (1 μg/mL) or IFN-γ (10 ng/mL). Nitric oxide (NO) production levels were determined in cell culture supernatant after 24 h for RAW264.7 cells or 20 h for MEF cells. In each case, 50 μL of the media was combined with 50 μL Griess Reagent (Promega). Absorbance was measured at 540 nm and compared with a calibration curve generated using the provided sodium nitrate standard.

### Example 12: PGE2 Assay

RAW264.7 cells were seeded into a 96 well format at 4×10⁴ cells/well and incubated overnight at 37° C. Cells were treated with a solvent control (DMSO, 1%, v/v) or serial dilutions of isolated compounds or extracts for 1 h before the addition of IFN-γ (10 ng/mL). Cells were incubated for an additional 24 h and the supernatant was analyzed for PGE₂ production using the Amersham Prostaglandin E2 Biotrak Enzyme immunoassay (EIA) system (GE Healthcare). The experiment was performed using the standard manufacturer's protocol.

### Example 13: Nqo1 Activity Assay

Wild-type (8×10³ cells/well), Nrf2−/− (8×10³ cells/well), and Keap1−/− (4×10³ cells/well) MEFs were seeded into a 96-well format and incubated overnight at 37° C. Cells were treated with a solvent control (DMSO, 1%, v/v) or serial dilutions of isolated compounds or extracts for 40 hours. The Nqo1 activity was measured using a previously described method (Prochaska and Santamaria, Analytical Biochemistry, Volume 169, Issue 2, March 1988, Pages 328-336.) Activities are relative to the vehicle control for the given cell line.

### Example 14: Oxidation of Cymopol (1) to Cymopol Quinone (5)

Silver oxide (14.2 mg) was added to a solution of cymopol 1 (6.6 mg) in dichloromethane (2.5 mL). The reaction mixture was stirred at room temperature for 15 min before being evaporated in vacuum. The crude product was purified by preparative TLC plate (ethyl acetate/hexane 1:4, v/v, R_{f} 0.8) to give 4.92 mg product 5 (75% yield). It should be noted that the product, cymopol quinone 5, is not stable in air, so should be handled quickly and stored in inert gas at low temperature.

¹H NMR (600 MHz, d₆-DMSO) δ ppm 7.50 (s, 1H), 6.64 (t, J=1.65 Hz, 1H), 5.10 (t, J=6.0 Hz, 1H), 5.02 (t, J=12.0 Hz, 1H), 3.03 (d, J=12.0 Hz, 2H), 2.06-2.02 (m, 2H), 2.02-1.98 (m, 2H), 1.61 (s, 3H), 1.55 (s, 3H), 1.52 (s, 3H) ppm.

¹³C NMR (150 MHz, d₆-DMSO) δ ppm 184.8, 179.8, 148.6, 138.9, 138.3, 136.4, 131.1, 131.0, 124.0, 118.3, 39.1, 27.0, 25.9, 25.5, 17.6, 15.8; DART-HRMS m/z 323.0634 ([M+H]⁺, C₁₆H₂₀BrO₂ calcd. 323.0641).

### Example 15: Zebrafish In Vivo Cell Migration Assays

Tg(mpx::GFP)^{ill4} transgenic zebrafish larvae (6 dpf) were pre-treated in duplicate with a solvent control ((DMSO, 1%, v/v) or serial dilutions of the compound or extract in 12-well plate format with five larvae per well containing 1 mL E2 medium containing 1 mM Tris pH 7.4. After 3 hours, larvae were anesthetised with Tricaine and tailfins were amputated using sharp blades to induce the migration of neutrophils. The larvae were then placed into fresh wells to be post-treated for an additional 3 hours. A fluorescence stereomicroscope was used to count the number of neutrophils that migrated to the site of injury. The assay was performed in triplicate to determine statistical significance.

### Example 16: RNA-seq Libraries Preparation Using CloneTech RiboGone, SMARTer Universal Low Input RNA Kit for Sequencing Combined with Illumina Nextera DNA Sample Preparation Kit

Illumina RNA library construction was performed at the Interdisciplinary Center for Biotechnology Research (ICBR) Gene Expression Core, University of Florida (UF). Quantitation was done on a NanoDrop Spectrophotometer (NanoDrop Technologies, Inc.), and sample quality was assessed using the Agilent 2100 Bioanalyzer (Agilent Technologies, Inc). rRNAs first were removed started with 90 ng of total RNA by ClonTech RiboGone-Mammalian-Low input ribosomal RNA removal kit for Human, Mouse and Rat Samples (cat #: 634848) following the manufacturer's protocol, then the depleted RNA were used for library construction with SMARTer Universal Low input RNA kit for sequencing (cat #: 634940) combined with Illumina Nextera DNA Library Preparation Kit (cat #: FC-121-1030) according to the user guide.

Briefly, 1^{st} strand cDNA is primed by a modified N6 primer (the SMART N6 CDS primer), then base-pairs with these additional nucleotides, creating an extended template. The reverse transcriptase then switches templates and continues transcribing to the end of the oligonucleotide, resulting single-stranded cDNA contains sequences that are complementary to the SMARTer oligonucleotide. The SMARTer anchor sequence and the N6 sequence serve as universal priming sites for DNA amplification by PCR for 10 cycles. Then Illumina sequencing libraries were generated with 125 pg of cDNA using Illumina Nextera DNA Sample Preparation Kit (Cat #: FC-131-1024) according to manufacturer's instructions. Briefly, 125 pg of cDNA was fragmented by tagmentation reaction and then adapter sequences added onto template cDNA by PCR amplification. Libraries were quantitated by Bioanalyzer and qPCR (Kapa Biosystems, catalog number: KK4824). Finally, the libraries were pooled equal molar concentration and sequenced by Illumina 2X75 NextSeq 500. Data are deposited in GenBank, accession number GSE107623.

### Example 17: NextSeq500 Sequencing: RNA seq

In preparation for sequencing, barcoded libraries were sized on the bioanalyzer, quantitated by QUBIT and qPCR (Kapa Biosystems, catalog number: KK4824). Individual samples were pooled equimolarly at 4 nM. This “working pool” was used as input in the NextSeq500 instrument sample preparation protocol (Illumina, Part #15048776, Rev A). Typically, a 1.3 pM library concentration resulted in optimum clustering density in our instrument (i.e., ˜200,000 clusters per mm 2) Samples were sequenced on a single flowcell, using a 2×75 cycles (paired-end) configuration. A typical sequencing run in the NextSeq500 produced 750-800 million paired-end read with a Q30>=85%. For RNA seq, 50-100 million reads provided sufficient depth for transcriptome analysis.

### Example 18: DSS-Induced Inflammation

The DSS model was performed with aged matched WT C57Bl/6 mice (6-8 weeks old). Dose optimization experiments were carried out with three concentrations (1.0, 1.5 and 2.0 mg/kg) using the formulation described above and otherwise identical conditions (daily treatment for 3 days followed by harvesting large intestines and cecum 12 h after the last dose). Nqo1 and Hmox1 mRNA levels were analyzed by RT-qPCR as described above. Based on this data, mice were then administered NP extract (2 g/kg body weight, daily) or vehicle (10% DMSO, 10% Cremophor) via oral gavage. After 3 days of pre-treatment, mice were given 3% DSS-drinking water ad libitum for 7 days along with daily extract administration. All animal procedures were performed according to the guidelines of the University of Florida Institutional Animal Care and Use Committee (IACUC). Mice were sacrificed and colon snips and stools were collected and snap frozen for further processing.

### Example 19: Lipocalin Immunoassay

Lipocalin-2 protein levels were detected using the DuoSet Mouse lipocalin-2 ELISA kit (R&D Systems). Briefly, frozen fecal pellets were weighed and resuspended in sterile PBS. The supernatant was collected and used for ELISA per application instructions. Data were normalized by stool weight.

### Example 20: RNA-seq Based Intestinal Microbiome Shift Analysis

Reads were quality filtered at Q20 and trimmed to remove remaining adaptors using Trimmomatic version 0.36 [Bolger A M, Lohse M, & Usadel B (2014) Trimmomatic: a flexible trimmer for Illumina sequence data. Bioinformatics 30(15):2114-2120]. Quality filtered and trimmed reads were aligned to iGenome Mus musculus GRCm38 reference genome using BWA version 0.7.16a and reads with alignments were excluded from further analysis [Li H & Durbin R (2010) Fast and accurate long-read alignment with Burrows-Wheeler transform. Bioinformatics 26(5):589-595]. Microbial classifications were assigned to the unaligned reads from the step above using centrifuge version 1.0.3 (59) (in pair-end mode utilizing both the forward and reverse reads together) and the bacteria and archaea database provided by the software developers (ftp://ftp.ccb.jhu.edu/pub/infphilo/centrifuge/data/p_compressed.tar.gz). Reads with archaeal taxonomy assignment were removed from subsequent analysis.

Moreover, the unaligned reads from above were used to generated close-reference OTUs at 97% similarity using Quantitative Insights into Microbial Ecology (QIIME) version 1.9.1 and the Green Gene reference dataset version 13.8 [Caporaso J G, et al. (2010) QIIME allows analysis of high-throughput community sequencing data. Nat Methods 7(5):335-336]. OTUs were generated from the forward reads and the reverse reads independently. Taxonomy assignment for the resulting OTUs was done in QIIME through the ribosomal database project (RDP) classifier after training on Green Gene reference dataset with confidence set to 50% [Wang Q, Garrity G M, Tiedje J M, & Cole J R (2007) Naive Bayesian classifier for rapid assignment of rRNA sequences into the new bacterial taxonomy. Appl Environ Microbiol 73(16):5261-5267]. Singleton OTUs were filtered out from the resulting OTU tables. The final OUT tables contained a minimum of U.S. Pat. Nos. 2,448,533 and 2,702,466 reads per sample for the forward and reverse ends, respectively. Those counts were then normalized and log₁₀ transformed using the following formula [McCafferty J, et al. (2013) Stochastic changes over time and not founder effects drive cage effects in microbial community assembly in a mouse model. ISMS J 7(10:2116-2125]:

$${log}_{10} 〈 ( \frac{Raw count}{Number of reads in sample} \times Average number of reads per sample ) + 1 〉$$

Principle Coordinate Analysis (PCoA) was generated from Bray-Curtis dissimilarity matrix obtained from the normalized and log₁₀ transformed OTU counts using phyloseq R package [McMurdie P J & Holmes S (2013) phyloseq: an R package for reproducible interactive analysis and graphics of microbiome census data. PLoS One 8(4):e61217; Team RC (2015) R: A Language and Environment for Statistical Computing. (R Foundation for Statistical Computing)].

Significant differences between the groups (Control, High and Low) were detected using the lm function in the R, using a linear model of the form:

variable ˜group+ε

where variable indicates either the PCoA axis or taxa (OTU, genus, family, order, class or phylum) normalized count (taxa present in at least 25% of the samples were only considered). An ANOVA analysis was conducted on the above model to generate a P-value for the group. All comparisons were done in pair-wise fashion (group: control vs. low, group: control vs. high and group: high vs. low). All p-values were adjusted for multiple hypothesis testing in R using the p.adjust function employing the method of Benjamini & Hochberg [Benjamini Y & Hochberg Y (1995) Controlling the false discovery rate: apractical and powerful approach to multiple testing. Journal of the Royal Statistical Society. Series B (Methodological) 57:289-300].

### Biological Results

Discovery of Potent ARE-Inducers from Cymopolia barbata

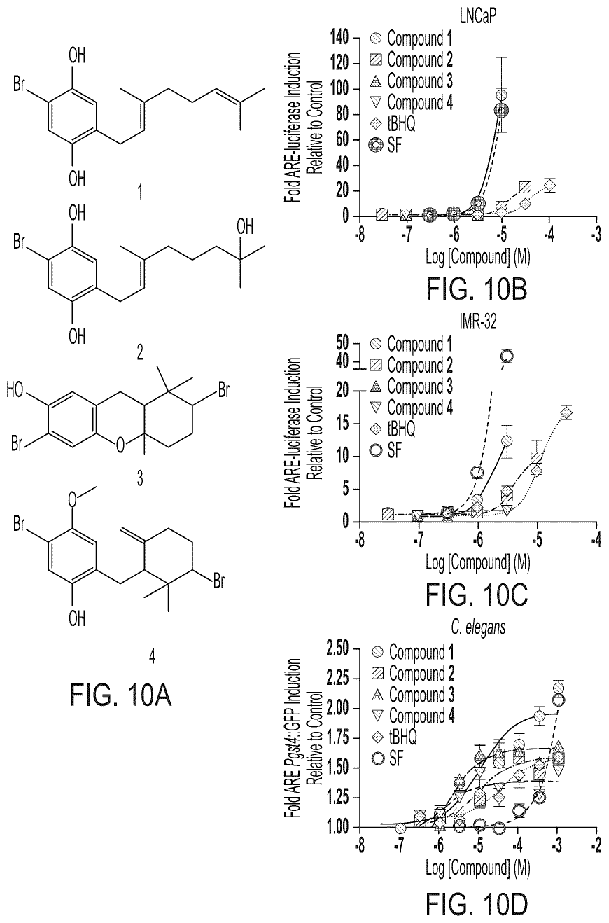

A luciferase reporter assay was used to guide fractionation of a subtropical marine alga, Cymopolia barbata, for activators of the ARE in various cell lines. The non-polar (NP) extract was liquid-liquid partitioned between hexane and 80% MeOH:H₂O v/v. The 80% solution was adjusted to 50% MeOH:H₂O v/v and partitioned against equal amounts of dichloromethane (DCM). The DCM fraction retained a majority of the mass and ARE-luc activity and was further fractionated. The DCM fraction was separated using size exclusion chromatography (Sephadex LH20 in 50% DCM:MeOH). Bioactive compounds from the 6th fraction were purified using reverse phase chromatographic methods, including a small-scale C18 column and several rounds of HPLC (LunaC18, MeOH/H₂O gradient). Four major compounds, collectively referred to as cymopols (cymopol (1, 8.50% yield), 7-hydroxy cymopol (2, 0.61% yield), cymobarbatol (3, 5.03% yield), and cyclocymopol monomethyl ether (4, 1.90% yield)) were isolated from an ARE-activating non-polar extract (FIG. 10a). The purified compounds activated ARE-luc in a dose-response manner in both LNCaP (FIG. 10b) and IMR-32 (FIG. 10c) cell lines, with cymopol (Compound 1) being the most active. Cymopols also dose-dependently induced a Pgst-4 reporter transgene in a whole animal model, C. elegans, as part of the biologically relevant SKN-1 pathway (FIG. 10d). Similarly, the SKN-1 pathway is cytoprotective with an electrophile/thiol sensitive repressor protein [Wang R, Paul V J, Luesch H. Seaweed extracts and unsaturated fatty acid constituents from the green alga Ulva lactuca as activators of the cytoprotective Nrf2-ARE pathway. Free Radic Biol Med. 2013; 57:141-153]. The activity of cymopols in C. elegans suggested that these compounds have sufficient bioavailability towards the antioxidant pathway.

Validation of Antioxidant Properties

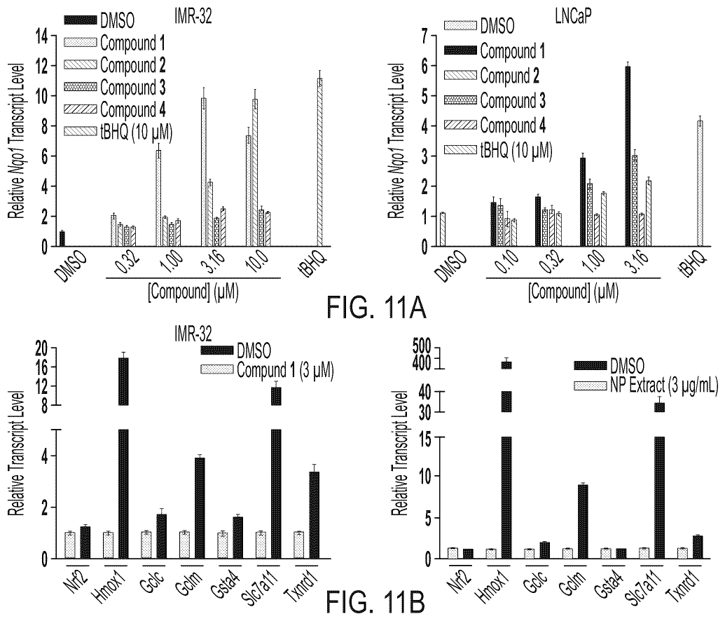

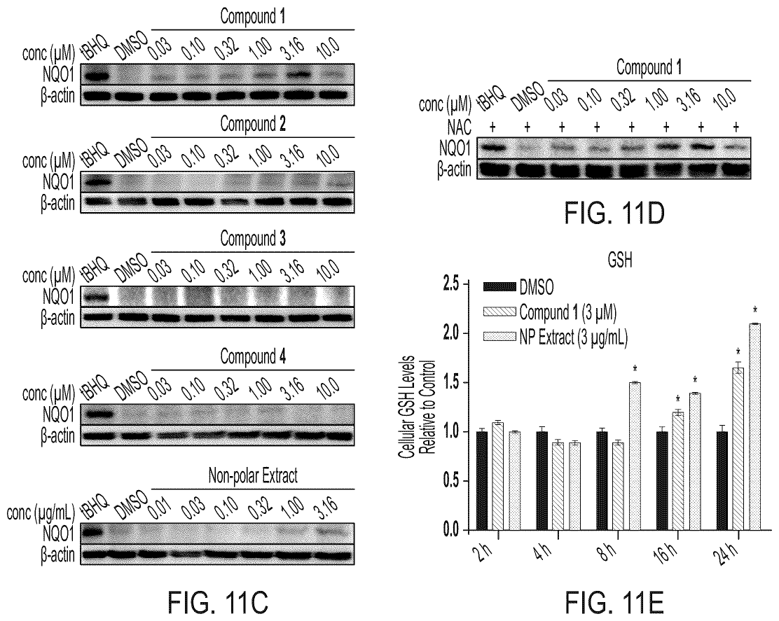

Compounds 1-4 and their parent fraction, the non-polar (NP) extract, were further analyzed in LNCaP and IMR-32 cell lines. The samples induced the transcription of the Nrf2/ARE regulated gene, NQO1, in a dose dependent manner in LNCaP and IMR-32 cells (FIG. 11a-c). Further evaluation of transcript regulation in IMR-32 cells by Compound 1 and the NP extract revealed that these samples induce the transcription of a series of ARE-driven genes, further confirming their bioactivity in the ARE/Nrf2 pathway (FIG. 11b), including GCLC (glutamatecysteine ligase, catalytic subunit), Gclm (glutamatecysteine ligase, modifier subunit), TXNRD1 (thioredoxin reductase 1), GSTA4 (glutathione S-transferase alpha-4), and HMOX1 (heme oxygenase 1). Of particular interest was an extreme transcript induction of an endogenous anti-inflammatory gene, Hmox1, most notably by the NP extract (360-fold in IMR-32 cells). The activation of this pathway and induction of Nqo1 and Hmox1 was further confirmed at the translational level using Western Blot (WB) analysis (FIG. 11c). In order to evaluate whether this rise in cytoprotective signalling products was strictly due to a toxic nature of the samples, cells were pre-treated with the anti-oxidant N-acetylcysteine (NAC), which is able to sequester free radicals, thus, minimizing toxic effects of applied compounds. The fact that Compound 1 was still able to induce the translation of NQO1 suggests that the stress response pathway was activated independently of a free radical toxicity mechanism (FIG. 11d). The natural products were able to increase cellular glutathione levels at 16 and 24 hours post-treatment (FIG. 11e), validating that the increase in genetic transcript levels of GSH synthetic machinery, GCLC and GCLM, leads to generation of a cellular product with cytoprotective activity.

Mechanism of Action

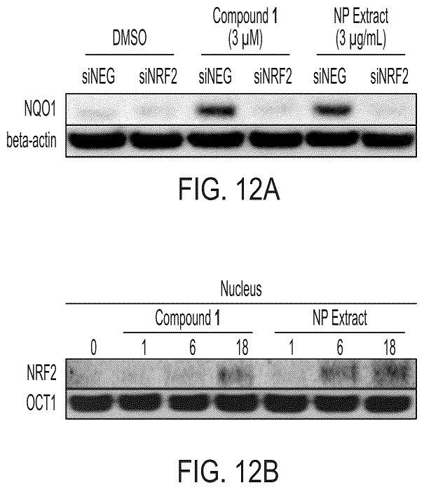

In order to determine whether NQO1 induction is Nrf2 dependent, IMR-32 cells were transfected with siRNA targeting Nrf2 or non-targeting siRNA. After 48 hours of RNAi of Nrf2, cells were treated with the samples or the vehicle control for 24 hours. The induction of NQO1 transcript levels was inhibited in siNrf2 transfected cells (FIG. 12a), indicating that Nrf2 is required for the induction of ARE-driven genes by Compound 1 and the NP extract. Nuclear protein was isolated from IMR-32 cells following treatment with Compound 1 or the NP extract for 0, 1, 6, or 18 hours to determine whether such treatment increases nuclear NRF2. Both Compound 1 and the NP extract induced nuclear translocation of NRF2, with the NP extract demonstrating increased levels after 6 h, and isolated Compound 1 after 18 h (FIG. 12b). This further confirms the mechanism of action of cymopols in the ARE/Nrf2 pathway.

Due to the hydroquinone structure of the compounds, it was hypothesized that the cymopols may act via alkylation of cysteine residues on Keap1 to induce the nuclear translocation of Nrf2. It was hypothesized that these compounds would undergo oxidation, forming the presumed bioactive quinone with a Michael acceptor capable of alkylating Keap1 cysteines. This theory was supported by reduced anti-oxidant efficacy of Compounds 3 and 4 relative to Compounds 1 and 2, which are likely more prone to redox cycling with the presence of a para-OH substitution pattern. The most active compound (1), which was also the major cymopol, was subjected to further analysis. In order to confirm the active form of the drug, Compound 1 was oxidized chemically to produce the corresponding cymopol quinone, generating compound 5 (FIG. 20). Mass tags that corresponded to m/z 485 and 483 for a conjugate addition reaction and m/z 405 and 407 for addition-elimination reaction of NAC with CymQ were detected (5). Additional adducts with two NAC molecules observed for mass tags corresponding to m/z 566 and 568 confirms multiple reactive sites for 5.

However, the cymopol quinone structure has three/four potentially reactive sites that could lead to Michael addition or addition-elimination since the bromide moiety could also act as a leaving group. To probe the chemical reactivity of cymopol quinone and cymopol, in vitro alkylation experiments were conducted in which both compounds were incubated with excess of NAC (or glutathione) for 2 h at room temperature and the resulting products were analysed by LC-MS. Various mass tags were observed when the quinone was used, while the hydroquinone, parent compound cymopol, lacked reactivity under these conditions, indicating that bioactivation to the quinone is required (FIG. 20).

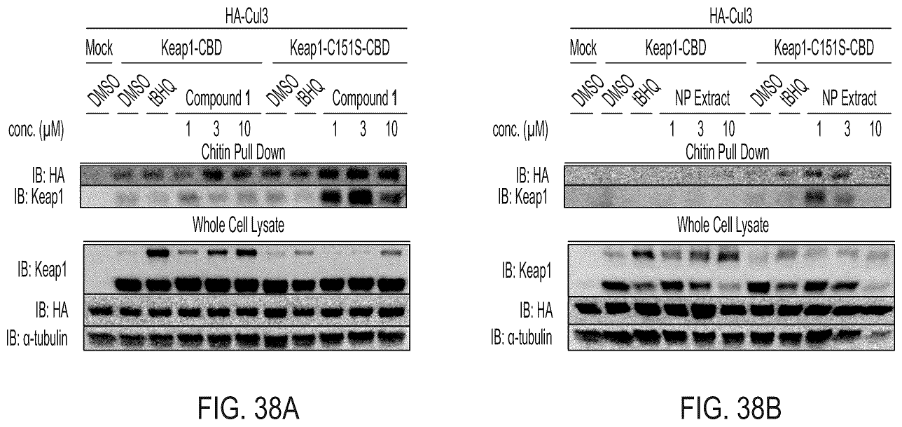

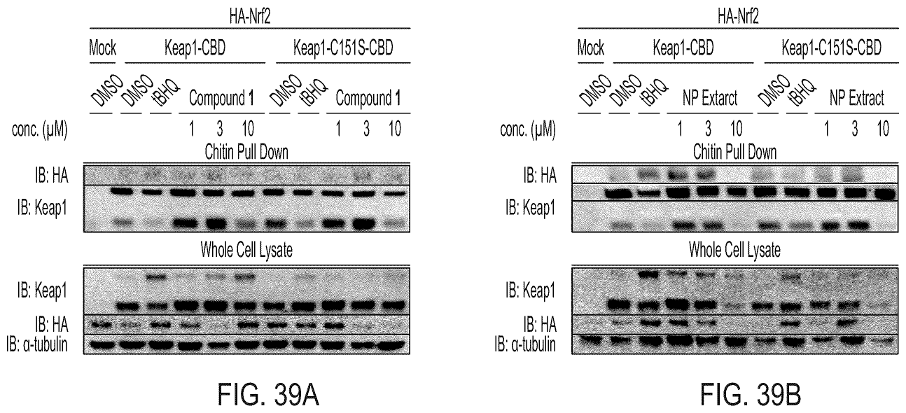

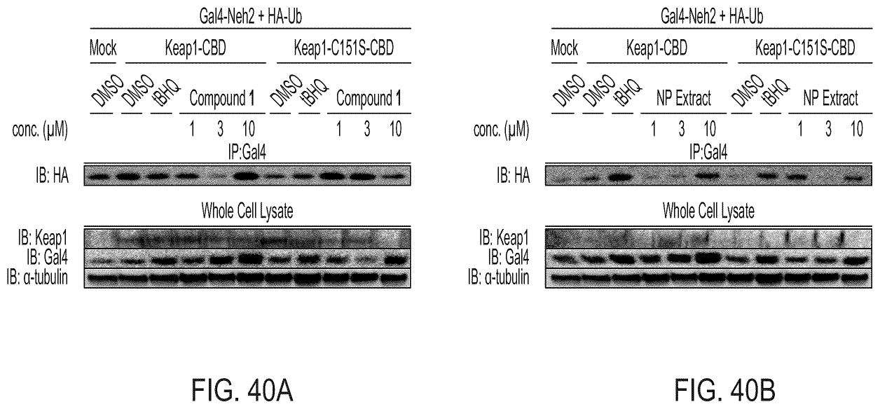

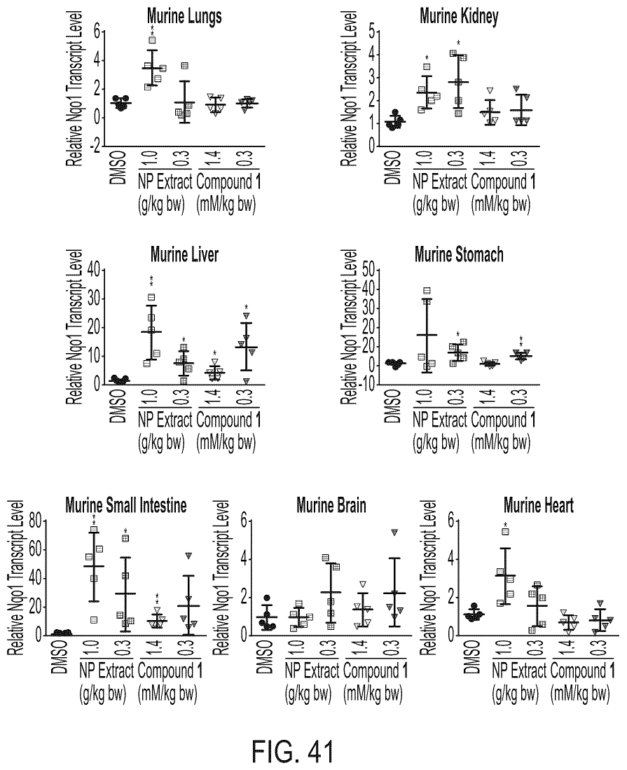

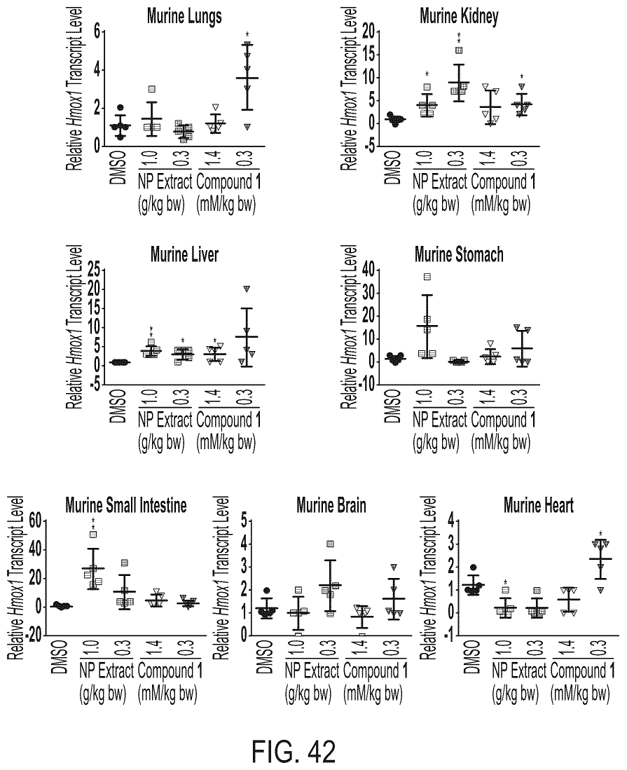

The reactivity at the amino acid level of KEAP1 was then investigated. Cymopol's cellular effect on the functionality if Keap1 as an adaptor for the Cul3-ubiquitin ligase complex was tested, in a similar fashion as described for AI-1 and AI-3 [Wang R, et al. (2013) In vitro and in vivo characterization of a tunable dual-reactivity probe of the Nrf2-ARE pathway. ACS Chem Biol 8(8):1764-1774; Hur W, et al. (2010) A small-molecule inducer of the antioxidant response element. Chem Biol 17(5):537-547]. In order to evaluate specific cysteine residues which may be alkylated in the presence of cymopols, HEK293 cells were co-transfected with either a mock plasmid, a wild-type Keap1-CBD, or a Keap1-C151S-CBD and HA-Cul3 (to evaluate Cul3 binding inhibition upon cymopol addition), HA-Nrf2 (to evaluate Nrf2-Keap1 interactions), or Gal4-Neh2 and HA-Ub (to evaluate ubiquitination patterns of Nrf2). Preliminary data suggest that compound 1 and the NP extract function at least in part through modification at Cys-151 of Keap1 as less Neh2 ubiquitination was observed and higher levels of Neh2 in the wild-type Keap1 were observed upon addition for compound 1 (FIGS. 38-40). Additionally, preliminary evaluation suggests that the amounts of wild-type Keap1-associated Cul3 were reduced in the presence of compound 1.

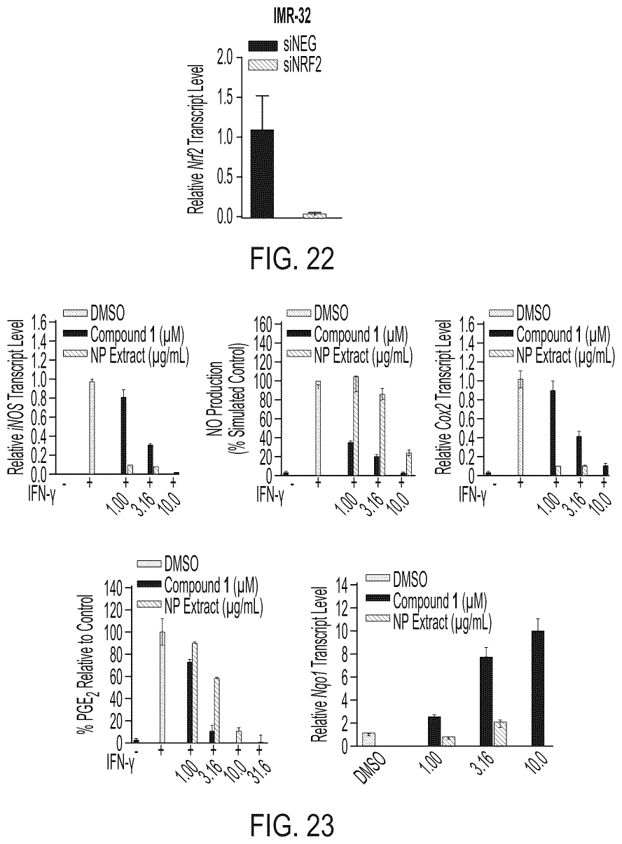

The corresponding hydroquinone/quinone pair (Compounds 1 and 5) were examined beside model compound tBHQ and its corresponding quinone for their ability to induce Nqo1 transcript levels in IMR-32 cells. In both cases, the quinones retained the bioactivity in a dose-dependent manner similar to that of the hydroquinone counterpart, supporting the theory that cymopols function via an α,β-unsaturated ketone (quinone) moiety (FIG. 21). In order to determine whether NQO1 induction is Nrf2 dependent, IMR-32 cells were transfected with siRNA targeting Nrf2 or non-targeting siRNA. After 48 h, Nrf2 transcript levels were reduced by 96% (FIG. 22). At that time cells were treated with the samples or the vehicle control for 24 h.

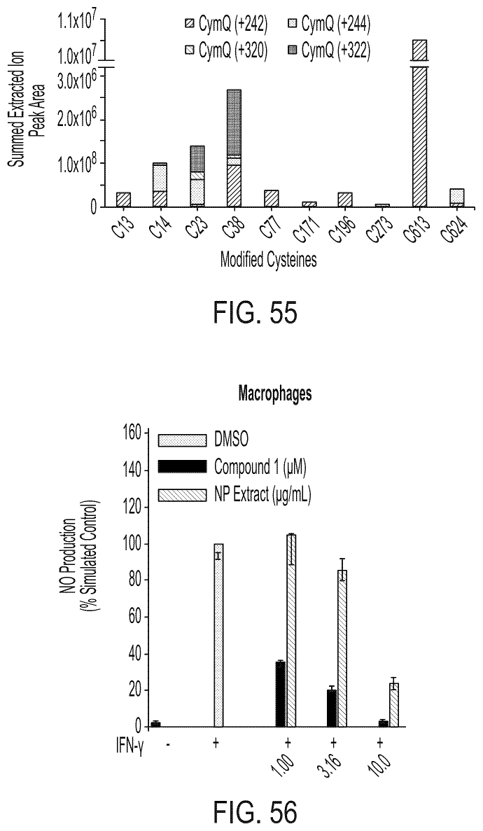

To obtain a global picture of cysteine modification of Keap1, rather than focusing on known functionally relevant residues, a proteomics approach was utilized, monitoring the effect of activated cymopol (CymQ) on cysteine residues of full-length human recombinant KEAP1 in vitro, similarly as described [Wang R, et al. (2013) In vitro and in vivo characterization of a tunable dual-reactivity probe of the Nrf2-ARE pathway. ACS Chem Biol 8(8):1764-1774; Hur W, et al. (2010) A small-molecule inducer of the antioxidant response element. Chem Biol 17(5):537-547]. Briefly, KEAP1 was treated with excess CymQ and adducts were mapped by LC-MS/MS in three separate experiments (five different conditions) using DTT, chymotrypsin and trypsin as the variables for different sample preparations, in order to capture more potential adducts. The most abundant modification observed was a mass increase of 242, resulting from an addition-elimination reaction of 5 (FIGS. 54 and 55) with loss of bromine, which is unique to CymQ compared with tBQ. Additional representative spectra for different types of tags can be found in SI Appendix, Fig. S4. Among the 10 cysteines modified by 5, only 2 cysteine residues (Cys23 and Cys 38) showed common adducts (244 and 322) under two different conditions (FIG. 54). These and other cysteine modifications were predominantly scattered across the N-terminal, BTB and C-terminal domains of the Keap1 protein. Interestingly, none of the highly reactive cysteines including Cys151 which is most consistently detected as being of high reactivity with many ARE activators failed to alkylate under the conditions we employed, which is likely due to reversibility of the reaction. Critical cysteines in Keap1 and their function have been categorized into six classes using a “cysteine code”. Most ARE activators belong to class 1 of this cysteine code where Cys151 is readily modified, although contradictory results are shown for these compounds based on the experimental conditions used. Since iodoacetamide used for free cysteine modification during sample preparation could cause a reversible effect of alkylated cysteines as demonstrated previously for sulforaphane, one possibility is that either CymQ-Cys151 is a reversible adduct or 5 is more favored towards alkylating different cysteines similar to other ARE activators. Although CymQ (5) has the capability to modify Keap1 and activate Nrf2, exactly which cysteines could act as the critical sensors is yet to be confirmed. Overall, it is clear the compound possesses polypharmacology at the cysteine level of Keap1 (and probably additional proteins), potentially reversible in nature, which will produce a net effect that translates into functional consequences.

Validation of Anti-Inflammatory Properties

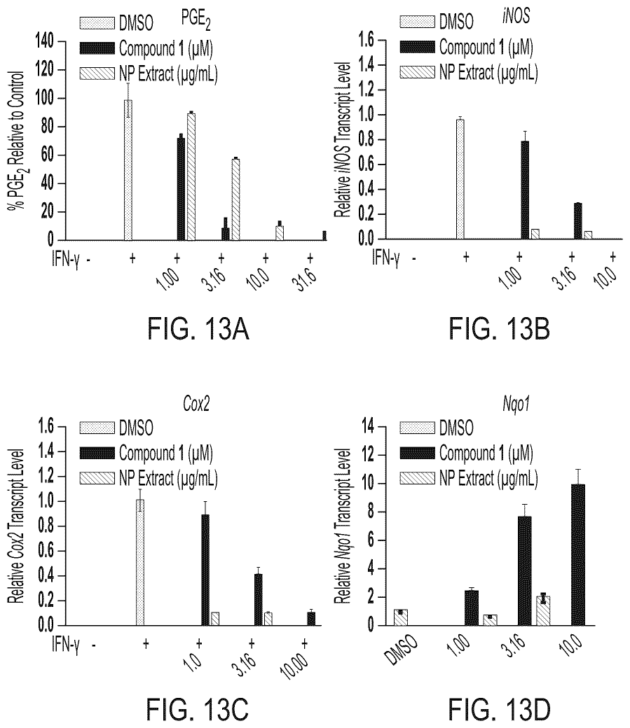

Due to the strong induction of Hmox1 transcript levels and the crosstalk between antioxidant (Nrf2) and anti-inflammatory (NFκB) pathways, the anti-inflammatory properties of Compound 1 and the NP extract were examined. RAW264.7 macrophage cells were pre-treated with IFN-γ to chemically induce the pro-inflammatory pathway. Compound 1 and the NP extract were then both added to the activated cells and effectively reduced the production of pro-inflammatory PGE2 (FIG. 13a). Furthermore, samples dose-dependently lowered the induced transcript levels of pro-inflammatory genes iNOS (FIGS. 13b and 56) (>96% by 1004 Compound 1 and >75% by 10 μg/ml NP extract) and Cox2 (FIG. 13c) (>90% by both 1004 Compound 1 and 10 μg/ml NP extract) following IFN-γ treatment, while simultaneously increasing antioxidant Nqo1 transcript levels by (>96% by 1004 Compound 1 and >75% by 10 μg/ml NP extract (FIG. 13d). In each case, the NP extract showed greater anti-inflammatory activities than its major component, cymopol, in human macrophage cells.

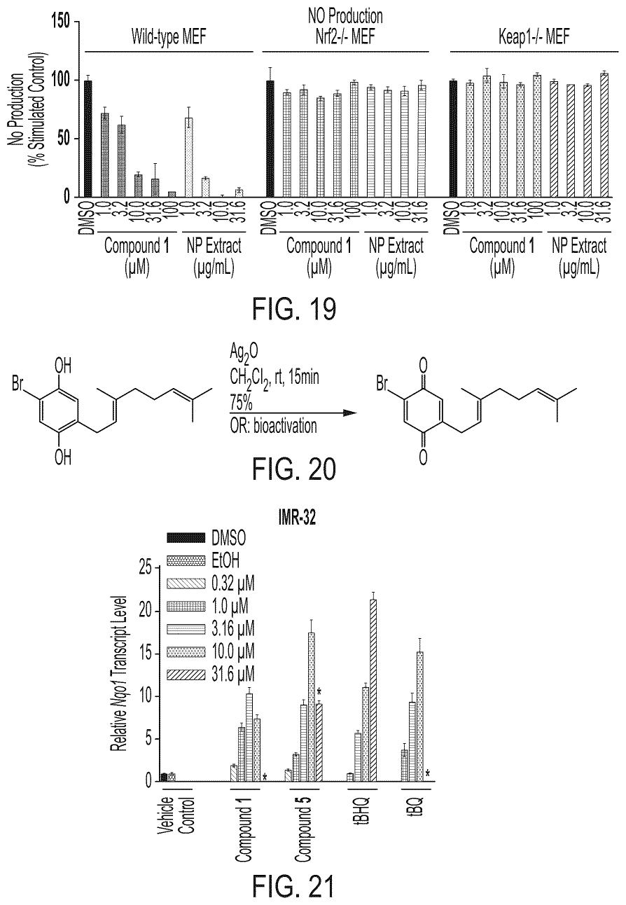

The Nqo1 activity and NO synthesis inhibition properties of the NP extract and cymopol (1) were evaluated in previously described murine embryonic fibroblast (MEF) cells [Dinkova-Kostova et al., PNAS Mar. 22, 2005 vol. 102 no. 12 4584-4589]. Compound 1 and the NP extract were able to induce Nqo1 enzymatic activity in a dose-dependent manner in each cell type relative to DMSO control. At high concentrations, compound 1 and the NP extract are able to induce relative Nqo1 activity more so in wild-type than in Nrf2 and Keap1 knockout MEFs at a given concentration. Compound 1 and the NP extract minimized NO production in wild-type MEFs stimulated with IFN-γ and TNF-α (FIG. 19). However, Compound 1 and the NP extract did not demonstrate appreciable anti-inflammatory effects in the Nrf2−/− and Keap1−/− MEFs, suggesting that the inflammatory effects function largely though an Nrf2/Keap1 mechanism of action in MEFs.

Due to the strong induction of Hmox1 transcript levels and the aforementioned crosstalk between antioxidant (Nrf2) and anti-inflammatory (NFκB) pathways, the anti-inflammatory properties of compound 1 and the NP extract were evaluated. RAW264.7 macrophage cells were pre-treated with IFN-γ to chemically induce the pro-inflammatory pathway [Lee D F, et al. (2009) KEAP1 E3 ligase-mediated downregulation of NF-kappaB signaling by targeting IKKbeta. Mol Cell 36(1):131-140; Nair S, Doh S T, Chan J Y, Kong A N, & Cai L (2008) Regulatory potential for concerted modulation of Nrf2- and Nfkb1-mediated gene expression in inflammation and carcinogenesis. Br J Cancer 99(12):2070-2082]. Compound 1 and the NP extract were then both added to the activated cells and effectively induced transcript levels of the pro-inflammatory genes iNOS, leading to the down-stream reduction in levels of NO by >96% in the presence of 10 μM Compound 1 and >75% in the presence of 10 μg/mL NP extract (FIG. 23). Additionally, both samples reduced the transcript levels of pro-inflammatory Cox2 (90% in the presence of both 10 μM Compound 1 and 10 μg/mL NP extract) (FIG. 23) following IFN-γ treatment, while simultaneously increasing antioxidant Nqo1 transcript levels by >96% in 10 μM Compound 1 and >75% μg/mL NP extract (FIG. 23). In each case, the NP extract showed greater anti-inflammatory activities than its major component (0.09%), cymopol, in mouse macrophage cells.

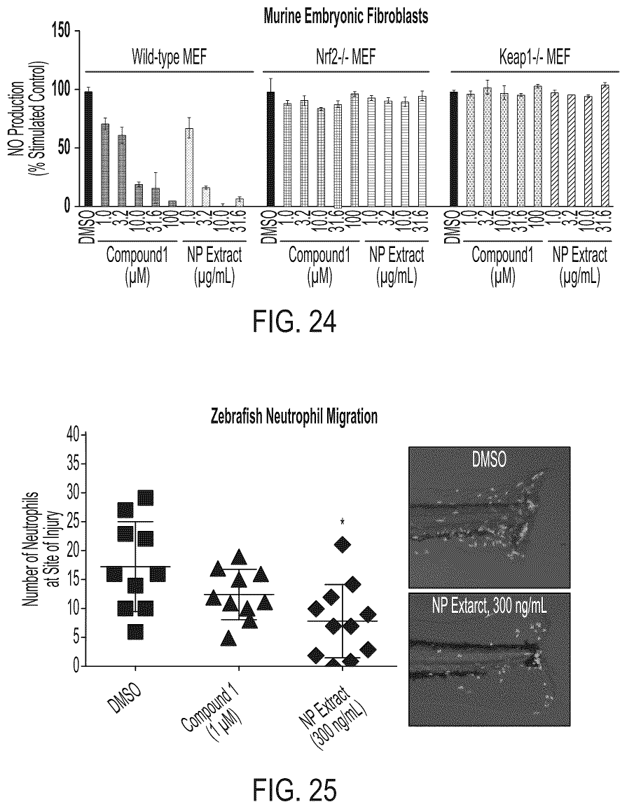

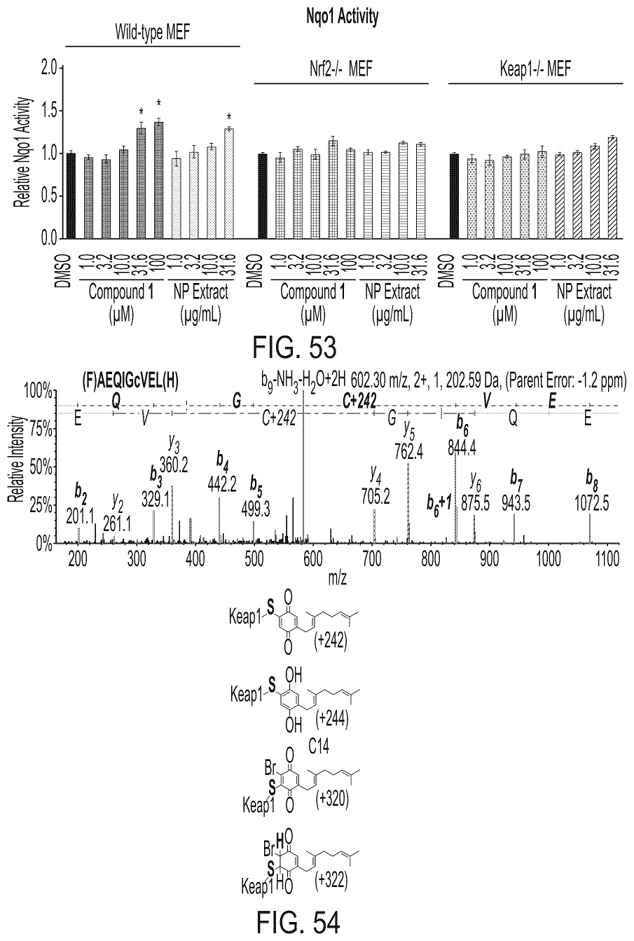

It was investigated whether cympols exert their anti-inflammatory function through the Nrf2/Keap1 pathway in biologically relevant murine embryonic fibroblast lines and whether it correlated with Nqo1 activity. The Nqo1 activity and NO synthesis inhibition properties of the NP extract and cymopol were evaluated in previously described murine embryonic fibroblast (MEF) cells [Dinkova-Kostova A T, Holtzclaw W D, & Kensler T W (2005) The role of Keap1 in cellular protective responses. Chem Res Toxicol 18(12):1779-1791]. At high concentrations, compound 1 and the NP extract were able to induce relative Nqo1 activity more so in wild-type than in Nrf2 and Keap1 knockout MEFs at a given concentration (FIG. 53). Compound 1 and the NP extract minimized NO production in wild-type MEFs stimulated with IFN-γ and TNF-α (FIG. 24). However, Nrf2−/− and Keap1−/− MEFs experienced no significant anti-inflammatory effect in the presence of the samples, suggesting that cymopols' inflammatory effects function largely though an Nrf2/Keap1 mechanism of action in MEFs.

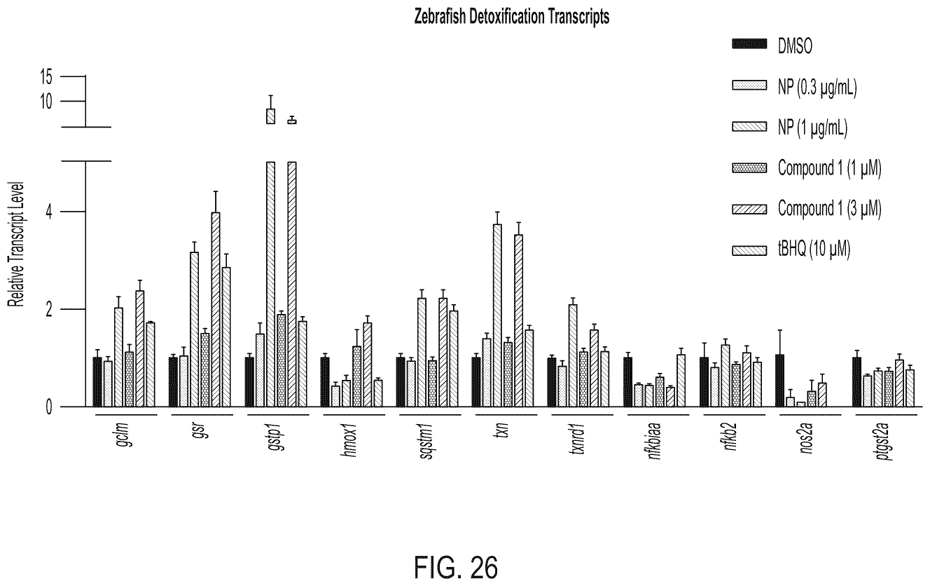

For in vivo evaluation, a previously reported neutrophil migration assay in the model organism, Danio rerio, was used [Wang X, et al. (2014) Inhibitors of neutrophil recruitment identified using transgenic zebrafish to screen a natural product library. Dis Model Mech 7(1):163-169]. In this assay, neutrophils migrate rapidly to a site of injury in response to a variety of chemo-attractants, including N-formyl methionine-leucine-phenylalanine (fMLF), interleukin-8, and ROS, providing an additional link between the ARE/Nrf2 pathway and the inflammatory pathway [Clark R A & Klebanoff S J (1979) Chemotactic factor inactivation by the myeloperoxidase-hydrogen peroxide-halide system. J Clin Invest 64(4):913-920; Ellett F, Pase L, Hayman J W, Andrianopoulos A, & Lieschke G J (2011) mpeg1 promoter transgenes direct macrophage-lineage expression in zebrafish. Blood 117(4):e49-56; Hattori H, et al. (2010) Small-molecule screen identifies reactive oxygen species as key regulators of neutrophil chemotaxis. Proc Natl Acad Sci USA 107(8):3546-3551; Lekstrom-Himes J A, Kuhns D B, Alvord W G, & Gallin J I (2005) Inhibition of human neutrophil IL-8 production by hydrogen peroxide and dysregulation in chronic granulomatous disease. J Immunol 174(1):411-417]. Control of the injury involves a reverse migration of neutrophils or macrophage engulfment following apoptosis [Mathias J R, et al. (2006) Resolution of inflammation by retrograde chemotaxis of neutrophils in transgenic zebrafish. J Leukoc Biol 80(6):1281-1288; Bratton D L & Henson P M (2011) Neutrophil clearance: when the party is over, clean-up begins. Trends Immunol 32(8):350-357]. Many human diseases, such as COPD and cystic fibrosis, consist of an uncontrolled neutrophilic activity and continued neutrophil migration to site of inflammation [Gernez Y, Tirouvanziam R, & Chanez P (2010) Neutrophils in chronic inflammatory airway diseases: can we target them and how? Eur Respir J 35(3):467-469]. In this assay, a transgenic zebrafish neutrophil-specific reporter line was used for an in vivo screen of natural products, which minimized neutrophil recruitment to an injury. A transgenic zebrafish line, Tg(mps::GFP)^{ill4}, contains neutrophils labelled with a green fluorescent protein (GFP) and has been previously used for screening natural product libraries for compounds which affect neutrophil migratory behavior. Several dose-response analyses were performed on zebrafish wild-type AB embryos in order to determine the concentration at which to treat the reporter line. A concentration was selected in which no phenotypic toxicity was noted in AB embryos treated 1.5 hpf through embryonic development. Additionally, due to the nature of the fin clip assay, any slight phenotypic toxicity will generally cause the tail to slightly degrade. A concentration was chosen at which both embryos and full adults demonstrated no phenotypic toxicity. Compound 1 and the NP extract did demonstrate toxicities at fairly low concentrations, leaving the therapeutic window in fish smaller than desired. Regardless, there was a therapeutic window and similar to what was seen in the cellular studies. 4dpf fish were pre-treated for 9 h with either a vehicle control, Compound 1, or the NP extract. The fish were anesthetized in tricaine before their fins were cut with a sharp razor blade. The fish were then quickly transferred into fresh media containing the treatments for an additional 3 h before the GFP-tagged neutrophils were counted under the fluorescent microscope. The NP extract demonstrated anti-inflammatory properties relative to a DMSO vehicle control with a statistically significant decrease in the number of neutrophils at the cut site (p<0.05, FIG. 25). The overall number of neutrophils which flood into the tail post-injury and those which make it to the cut site in fish pre-treated with the NP extract were lesser than those in the DMSO treated. Both samples demonstrated the ability to induce detoxification enzymes in zebrafish (FIG. 26).

In Vivo Validation of Anti-Oxidant and Anti-Inflammatory Activity in Mice

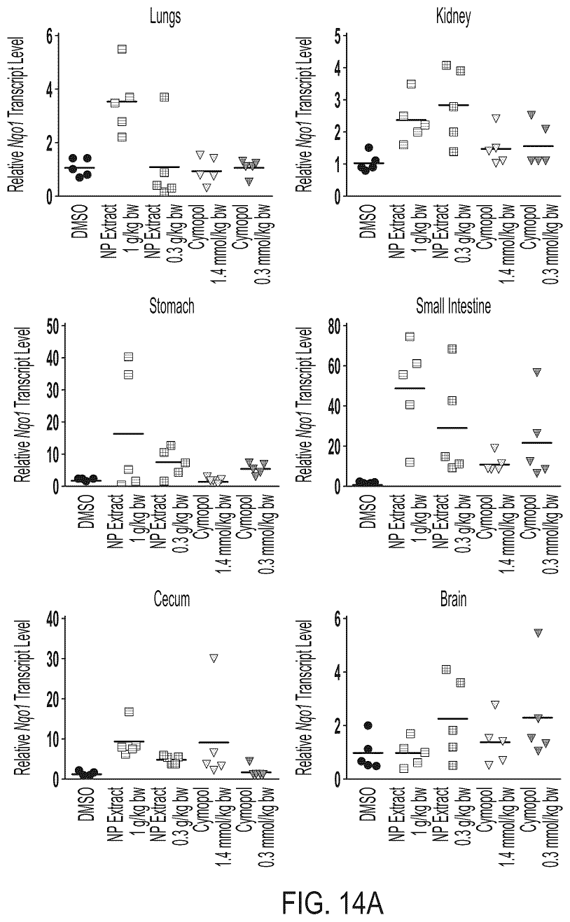

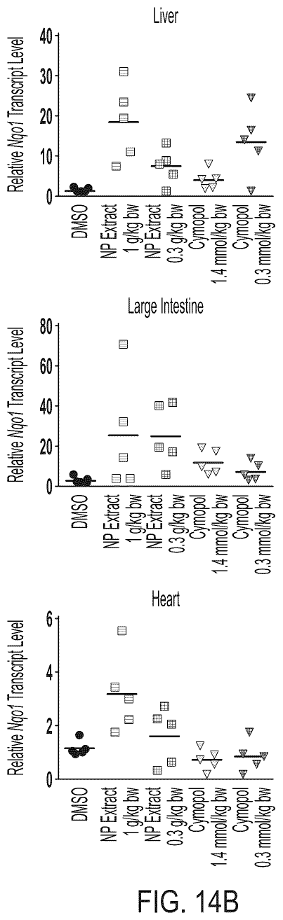

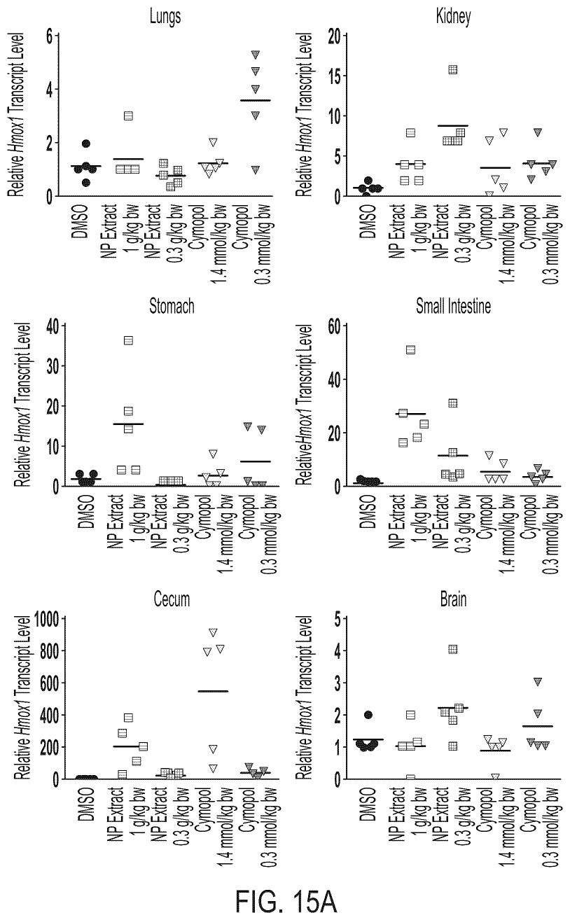

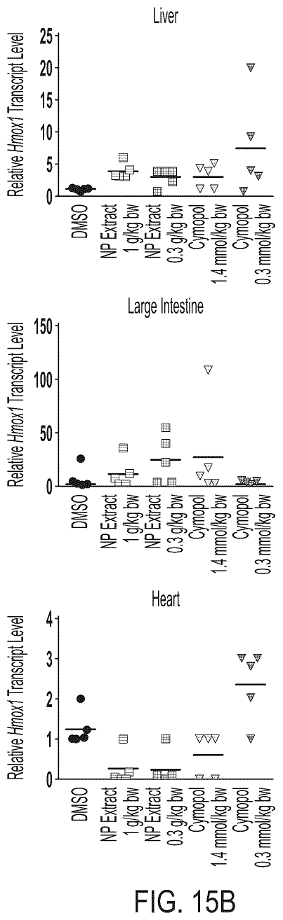

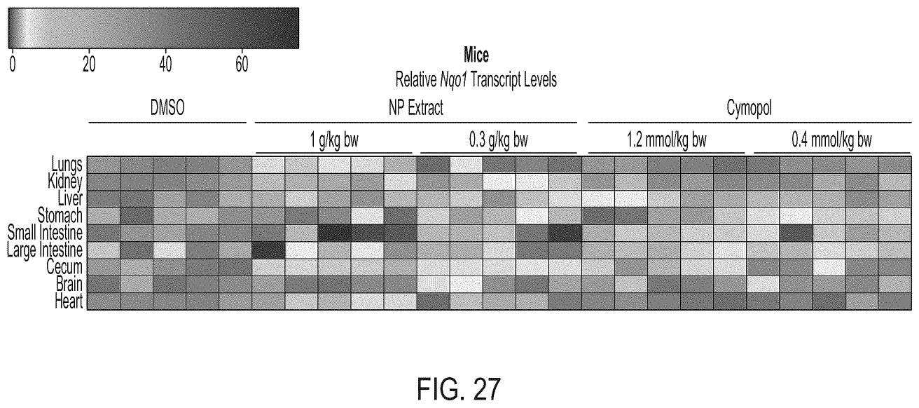

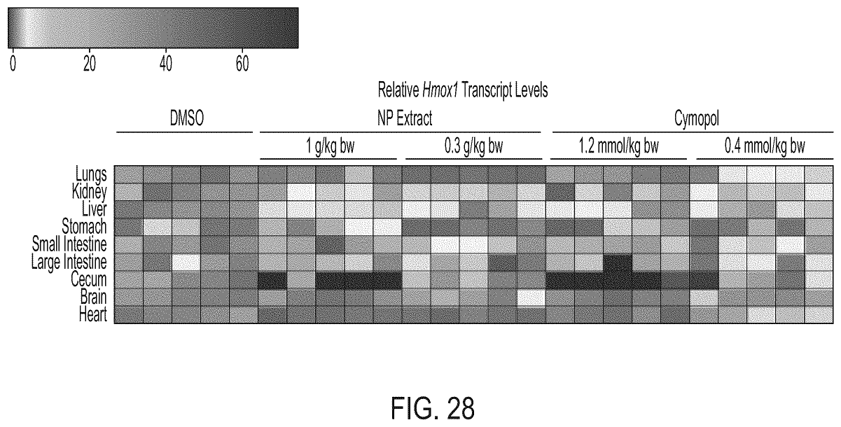

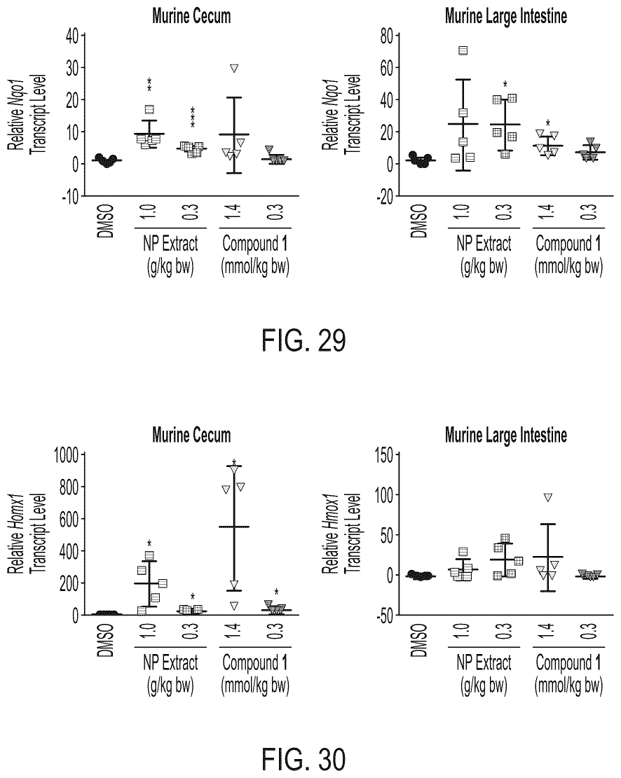

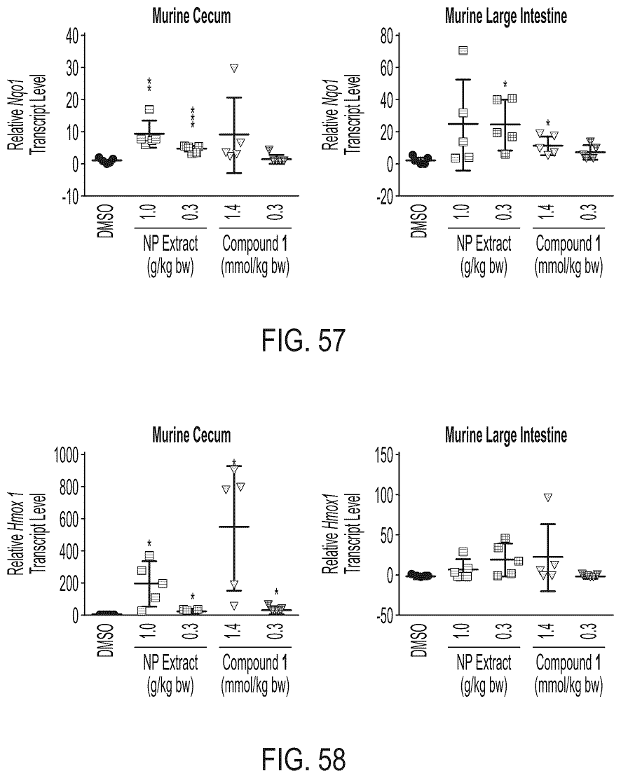

A low dose and a high dose of cymopol (1) or the NP extract were administered to 4-week-old male mice via oral gavage. Concentrations for the doses were determined based on animal studies using a chemically and functionally similar quinone structure, tBHQ, which can be dosed with no toxicity at 200 mg/kg bw (1.2 mmol/kg bw) (WHO Food Additive Series 40). In order to evaluate biological activities, including potential differences in biological availability, a concentration of the NP extract (0.3 g/kg bw) that was functionally equivalent to a concentration of Compound 1 as determined by transcriptional Nqo1 data in IMR-32 cells was administered. Compound 1 was also tested at ⅓ of its concentration (0.3 g/kg bw) for the low dose. A 3-fold higher dose of the NP extract (1 g/kg bw) was also evaluated to determine if any further enhancement in activity could be attained. Mice were treated by oral gavage for 3 consecutive days every 12 hours and were euthanized 12 hours following the last treatment. Nine organs were isolated for evaluation of Nqo1 (FIGS. 14 and 27) and Hmox1 (FIGS. 15 and 28) transcript levels. Consistent with the theory of extract bioavailability, wherein a crude extract may have greater in vitro and/or in vivo activity than an isolated active constituent at the equivalent dose [Rasoanaivo, P, Wright C W, Willcox, ML, Gilbert, B. (2011) Whole plant extracts versus single compounds for the treatment of malaria: synergy and positive ineractions.'Malar J, 10(Suppl 1):S4], the cellular functional equivalent of the NP extract (low dose, 0.3 g/kg bw) showed greater activity in all organs than the purified cymopol (Compound 1, 1.2 mmol/kg bw). Of particular interest was a large increase in the anti-inflammatory and anti-oxidant genes within the digestive tract, with the large intestine, small intestine, and cecum showing the highest induction levels of Nqo1 and Hmox1 (FIGS. 57 and 58).

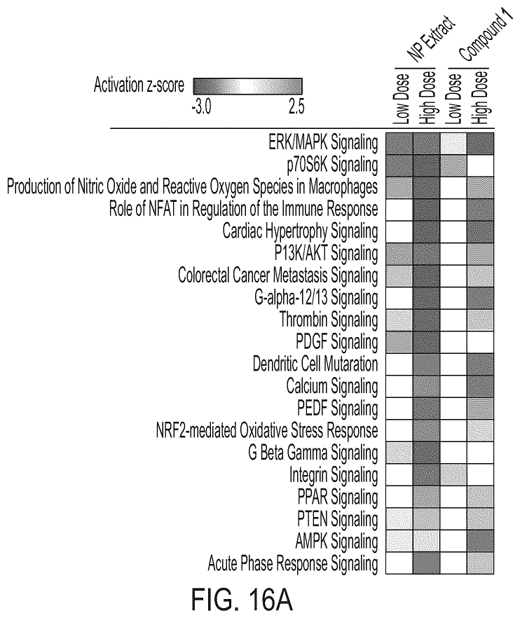

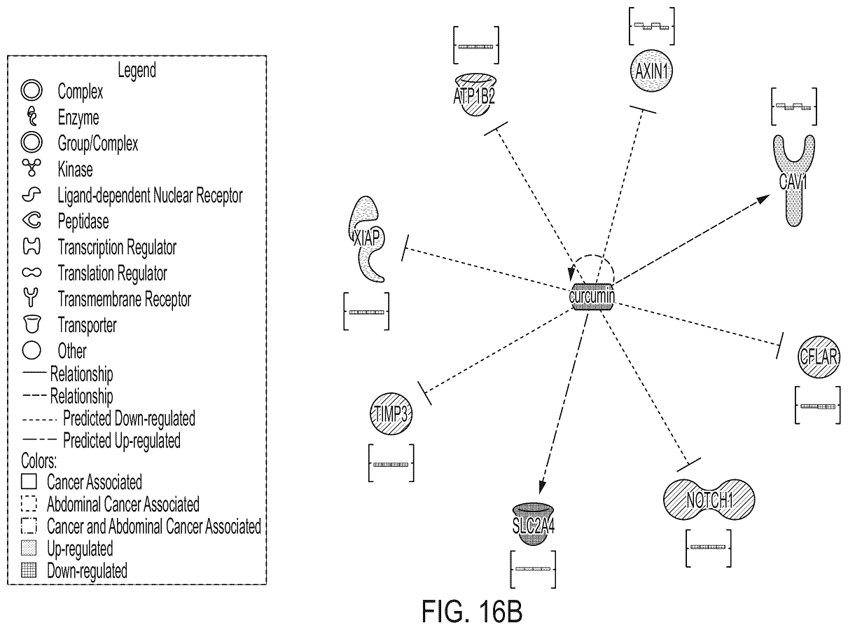

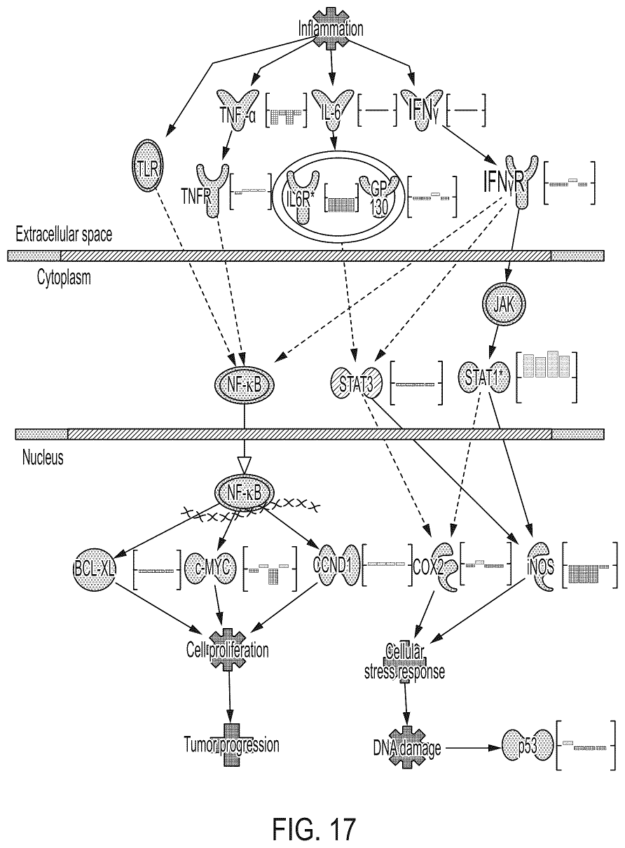

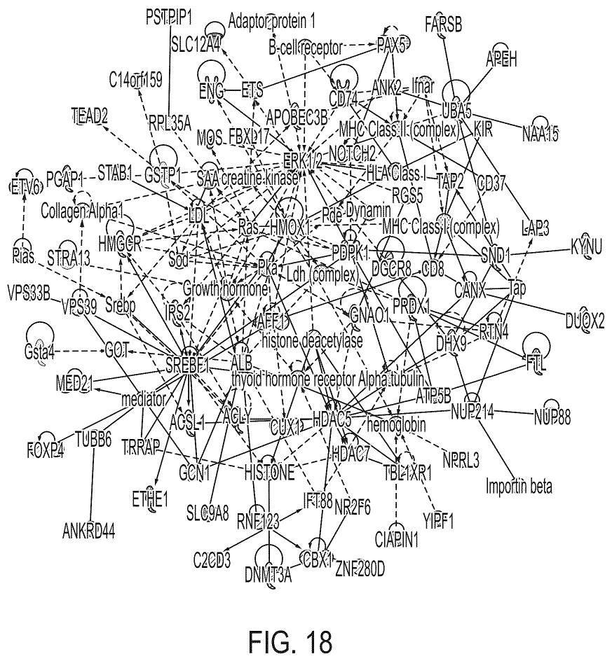

To further evaluate the effect of Compound 1 and the NP extract, the large intestine was examined using RNAseq. Genes were prioritized based on those with a 1.5 fold change in transcript level with p<0.05. The web-based analysis tool, IPA, was used to further investigate the effect of the cymopols on the large intestine. Consistent with our in vitro and in vivo modelling systems, multiple canonical pathways associated with antioxidant and anti-inflammatory (FIG. 16a) were observed. As anticipated for activators of the ARE/Nrf2 pathway (e.g., any compound, seaweed extract, or enriched seaweed extract presented herein), the canonical pathway entitled ‘NRF2-mediated Oxidative Stress Response’ appeared within the top 20 canonical pathways identified in the IPA comparison analysis. Furthermore, the ERK/MAPK signalling pathway was down-regulated, which indirectly stabilizes NRF2 in mammalian systems [Keum Y S, Yu S, Chang P P, Yuan X, Kim J H, Xu C, et al. Mechanism of action of sulforaphane: inhibition of p38 mitogen-activated protein kinase isoforms contributing to the induction of antioxidant response element-mediated heme oxygenase-1 in human hepatoma HepG2 cells. Cancer Res 2006; 66:8804-13]. Additionally, an appreciable reduction in the canonical pathway, ‘Production of Nitric Oxide and Reactive Oxygen Species in Macrophages’, was observed, which supports earlier findings that Compound 1 and the NP extract possess both anti-inflammatory and antioxidant properties. Furthermore, an overall decrease in ‘Colorectal Cancer Metastasis Signalling’ was observed. Interestingly, a decrease in the PI3K/AKT signalling pathway coupled with an increase in PTEN signalling supported this finding, as PI3K/AKT induces translation of HIF-1α from mRNA to the protein, while PTEN inhibits this process via dephosphorylation of PI3K products. These findings indicate that the cymopols may show great potential in the treatment or prevention of colorectal cancers. One interesting finding is that Compound 1 and the NP extract function quite similarly to curcumin, a natural product well characterized for its function in the prevention of inflammation, cancer, and neurological diseases (FIG. 16b) [Mahmood, K, Mahmood Zia, K, Zuber, M, Salman, Mahwish, Naveed, Anjun M. (2015) Recent developments in curcumin and curcumin based polyperic materials for biomedical applications: a review. Int J Biol Markers, 81:877-890]. The blue lines indicate genes which are down-regulated by curcumin, whereas the orange lines correspond to genes which are up-regulated. The RNAseq data is displayed as expression bar charts (NP Low dose, NP high dose, Compound 1 low dose, and Compound 1 high dose, respectively). Green bars indicate genes which have been down-regulated while red bars indicate genes which are upregulated. Curcumin has been known to target colorectal cancer stem cells and reduce tumor occurrence via pathways such as the Wnt/β-catenin, Sonic Hedgehog, Notch and PI3K/Akt/mTOR signalling pathways. While no notable difference in the Wnt/β-catenin or Sonic Hedgehog signalling was observed for the treatment with Compound 1 and the NP extract, the PI3K/Akt/mTOR pathway was greatly affected, demonstrating targeting and selectivity differences between curcumin and cymopols in their abilities to affect colon health. Further exploring the ‘Colorectal Cancer Metastasis Signalling’ (FIG. 17) shows a portion of this canonical pathway, which highlights the potential for cymopols in treating and/or preventing colorectal diseases. Elevated levels of TNF at sites of inflammation have been associated with the pathogenesis of these chronic inflammatory diseases. This concept has been supported by several studies including a transgenic mice study that demonstrated that overexpression of TNF led to the development of arthritis, similar to that of rheumatoid arthritis (RA) patients [Keifer, J., Probert, L., Cazlaris, H. Georgopoulos, S., Kaslaris, E/, Kioussis, D., et al. (1991). Transgenic mice expression human tumour necrosis factor: a predictive genetic model of arthritis. EMBO J 10, 4025-4031]. In 1992, it was demonstrated in collagen-induced arthritic murine models that anti-TNF agents were able to attenuate the disease. Later, the beneficial use of cA2, a chimeric monoclonal antibody (now called infliximab), was reported for patients with RA [Elliot M F, Maini R N, Feldman M, Long-Fox A, Charles P, Katsikis P, Brennan F M, Walker J, Bijl J, Ghayeb J (1993) Efficacy of B-cell-targeted therapy with rituximab in patients with theumatoid arthritis. NEJM (36) 1681-90]. In recent years, discovery of TNF-antagonists has been of great interest for the treatment of rheumatoid arthritis and other chronic inflammatory diseases, such as Crohn's Disease. Transcript levels of TNFα are down-regulated at the low dose of Compound 1 and the NP extract. A decrease in interleukin-6 receptor transcription supports the observed overall decrease in the pro-inflammatory response resulting in a decrease in transcript levels of oncogenic c-Myc. Furthermore, a decrease in the oncogenic Stat3 as well as reduced Cox2 levels results in a decrease in DNA damage associated with cellular stress responses. STAT3 is important for cellular responses to stimuli such as TNFα and is associated with an increase in ROS formation. A decrease in STAT3 is also important in reducing inflammation, as it has been shown to facilitate the nuclear accumulation of NFκB [Zouein F A, Duhe R J, Aran I, Shirey K, Josler J P, Liu J, Saad I, Kurdi M, Booz G W. (2014) Loss of STAT3 in mouse embryonic fibroblasts reveals its Janus-like actions on the mitochondrial function and cell viability. Cytokine, 66:7-16]. A modest induction of STAT1 is also seen in the ‘Colorectal Cancer Metastasis Signalling’ pathway. STAT1 activation has been associated with antitumor properties, including suppression of tumor proliferation, induction of apoptosis, and inhibition of angiogenesis [Huang S, Bucana C D, Van Arsdall M, Fidler I J. Stat1 negatively regulates angiogenesis, tumorigenicity and metastasis of tumor cells. Oncogene. 2002; 21:2504-2512; Stephanou A, Latchman D S. STAT-1: a novel regulator of apoptosis. Int J Exp Pathol. 2003; 84:239-244; Hosui A, Klover P, Tatsumi T, Uemura A, Nagano H, et al. Suppression of signal transducers and activators of transcription 1 in hepatocellular carcinoma is associated with tumor progression. Int J Cancer. 2012; 131:2774-2784; Bromberg J F, Horvath C M, Wen Z, Schreiber R D, Darnell J E Jr. Transcriptionally active Stat1 is required for the antiproliferative effects of both interferon alpha and interferon gamma Proc Natl Acad Sci USA. 1996; 93:7673-7678; Battle T E, Lynch R A, Frank D A. Signal transducer and activator of transcription 1 activation in endothelial cells is a negative regulator of angiogenesis. Cancer Res. 2006; 66:3649-3657]. To evaluate the most active treatment in cancer pathways, a network was generated by overlaying top networks associated with cancer using the high dose of the NP extract (FIG. 18). Genes associated with ‘cancer’ are outlined in black, while those specifically associated with ‘abdominal cancer’ are shown in blue. Those associated with both general ‘cancer’ and ‘abdominal cancer’ are outlined in red and make up a majority of the network. Strong up-regulation of ARE-driven cytoprotective enzymes HMOX1, GSTA4, and GSTP1 is observed, which supports the earlier findings of their induction in cell culture, zebrafish, and C. elegans (FIGS. 25 and 26). Collectively, these results indicate the promising properties of cymopols for the prevention and treatment of cancer caused by oxidative stress chronic inflammatory disease in the digestive tract. One particular application is the prevention and/or treatment of Crohn's disease, in which TNFα mediates chronic inflammation. Of course, it should be noted that, as with any stress response pathway, the regulation needs to be tightly controlled. However, based on our analyses in vitro and in vivo, cymopols offer great potential as mediators of chronic inflammation and oxidative stress associated with the development of diseases such as colorectal carcinoma.

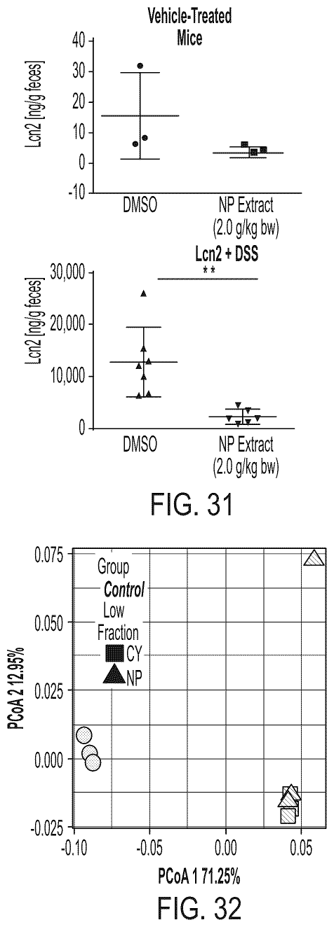

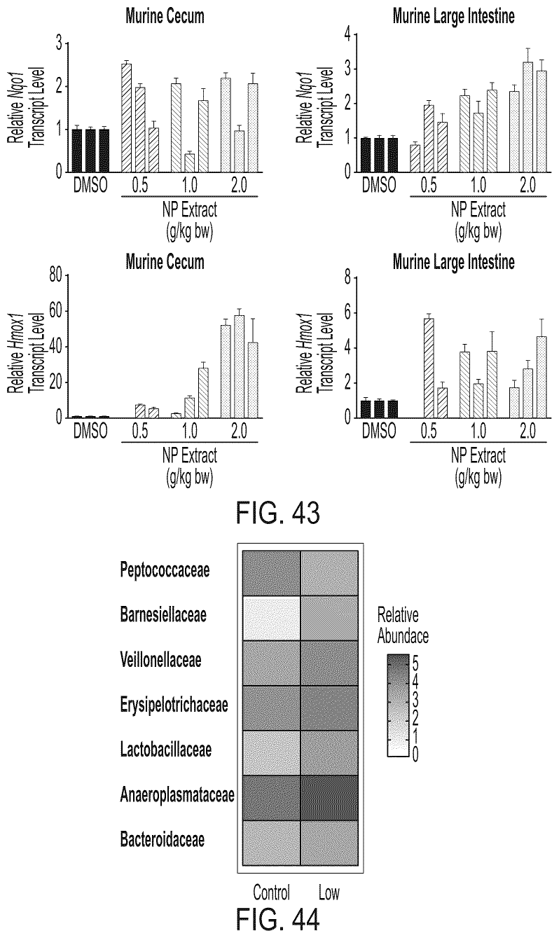

It was explored whether the transcriptional response leads to potentially disease-modifying readout. There is evidence that bioavailable Nrf2 activators can attenuate DSS-induced colitis in this acute chemical model [Liu X, et al. (2016) Dimethyl fumarate ameliorates dextran sulfate sodium-induced murine experimental colitis by activating Nrf2 and suppressing NLRP3 inflammasome activation. Biochem Pharmacol 112:37-49; Wang Y, et al. (2016) 3-(2-Oxo-2-phenylethylidene)-2,3,6,7-tetrahydro-1H-pyrazino[2,1-a]isoquinolin-4(1 1bH)-one (compound 1), a novel potent Nrf2/ARE inducer, protects against DSS-induced colitis via inhibiting NLRP3 inflammasome. Biochem Pharmacol 101:71-86; Xi M Y, et al. (2013) 3-aroylmethylene-2,3,6,7-tetrahydro-1H-pyrazino[2,1-a]isoquinolin-4(11bH)-ones as potent Nrf2/ARE inducers in human cancer cells and AOM-DSS treated mice. J Med Chem 56(20):7925-7938]. Conversely, Nrf2−/− mice are more susceptible to colitis-associated colorectal cancer [Khor T O, et al. (2008) Increased susceptibility of Nrf2 knockout mice to colitis-associated colorectal cancer. Cancer Prev Res (Phila) 1(3):187-191]. Thus, it was tested whether the Cymopolia extract was able to reduce inflammation in a mouse model of DSS-induced colitis by measuring levels of the inflammatory marker Lipocalin 2 (Lcn-2) [Chassaing B, et al. (2012) Fecal lipocalin 2, a sensitive and broadly dynamic non-invasive biomarker for intestinal inflammation. PLoS One 7(9):e44328]. As the mice (6-8 weeks old) in this study were older than the mice used for the tissue distribution and transcriptional assays, dose optimization studies were performed using the same conditions as above (3 days daily treatment), indicating that a slightly higher dose (2.0 g/kg) was required in these older mice to induce a similar robust response in the large intestines and cecum (FIG. 43). Mice were pretreated with extract (2.0 g/kg) or vehicle for 3 days prior to and during DSS administration for a total of 10 days. Lcn-2 level was significantly reduced (p=0.0012) in extract-gavaged, DSS exposed mice compared to Vehicle treated mice, while there was a trend that baseline Lcn-2 is reduced as well (FIG. 31).

RNA-seq Based Intestinal Microbiome Analysis

Cymopol 1 (CY) and the corresponding extract (NP) also have antibacterial activity with undefined spectrum. In preliminary testing, it at least inhibited Staphylococcus aureus and Bacillus cereus. Thus, a potential change in the intestinal microbial composition was anticipated, particularly if the extract has differential antibacterial activity.

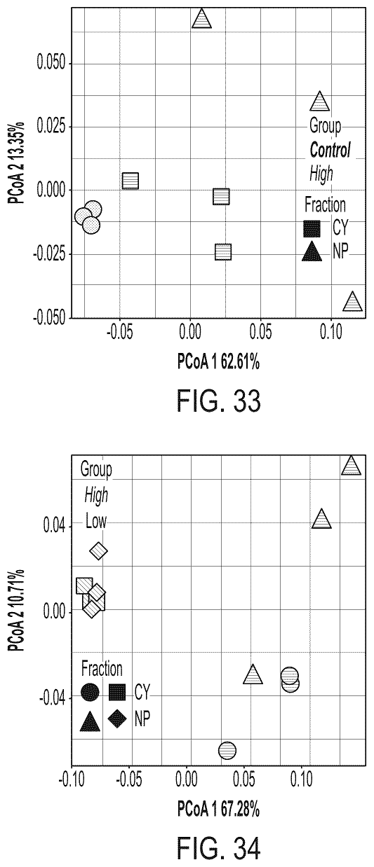

The RiboGone kit used for the RNA-seq library preparation only removes the rRNAs from mammalian cells but not bacterial rRNA. It was noticed that most RNA-seq reads were of bacterial origin and, thus, it was desirable to take advantage of the opportunity to investigate possible effects of cymopol and the extract on the intestinal microbiota. The bacterial sequences present in the RNA-seq data were classified using two independent pipelines: centrifuge [Kim D, Song L, Breitwieser F P, & Salzberg S L (2016) Centrifuge: rapid and sensitive classification of metagenomic sequences. Genome Res 26(12):1721-1729] (which considered all the non-mouse reads) and QIIME close-reference (which considered only 16S bacterial sequences present in the dataset) [Caporaso J G, et al. (2010) QIIME allows analysis of high-throughput community sequencing data. Nat Methods 7(5):335-336]. Interestingly, mice treated with either the extract or Cymopol 1 showed different gut microbial composition than the control group (FIGS. 32 and 33), regardless of the fraction used. A significant shift in the microbiota between the low and the high groups was detected, also independent of the fraction at which they were administered (FIG. 34). No significant differences were detected between the fraction groups (CY low vs. NP low or CY high vs NP high).

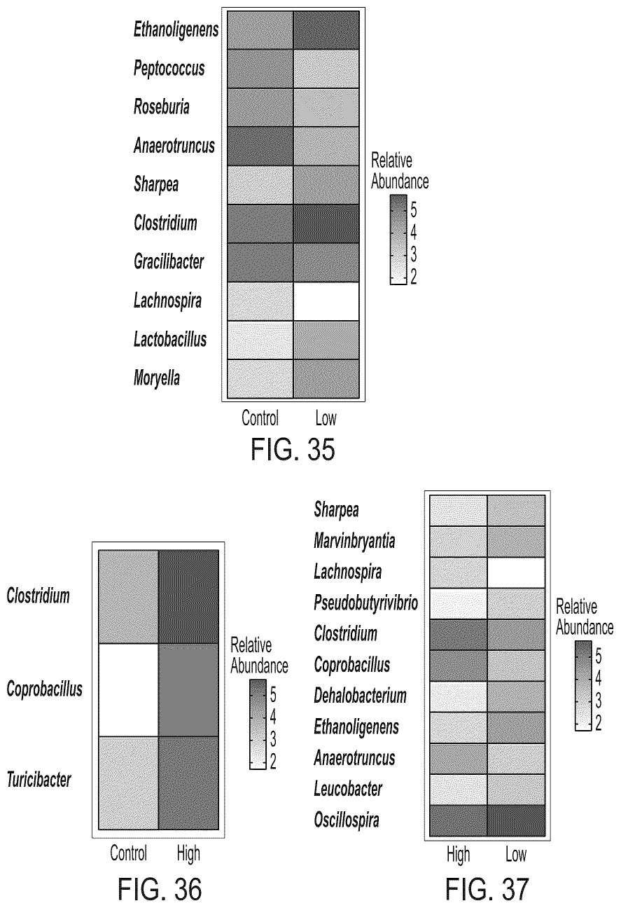

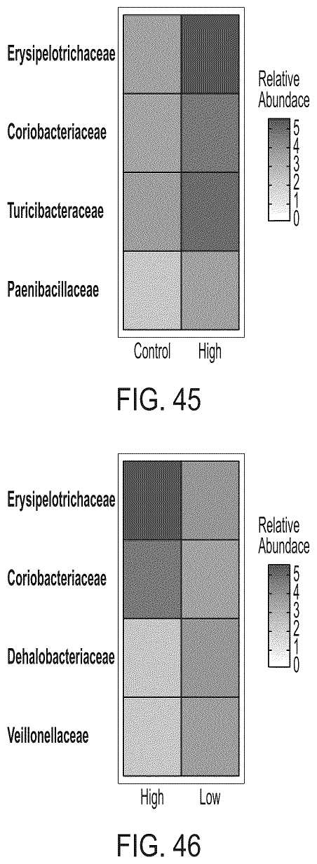

To characterize the shift in the microbiota, the genera that show significant difference between the three groups were examined. The largest shift was observed in the control versus the low groups (ten genera, FIG. 35) and the high versus the low groups (eleven genera, FIG. 37). On the other hand, the control versus the high groups elicited a modest effect with only three genera significantly different (FIG. 36). Further, the bacterial families that show significant (FDR p<0.05) differences between the three groups were examined. It was found that low versus control comparison showed the highest number of change. A total of 7 bacterial families show changes in their relative abundance. Interestingly, all except one family (Peptococcaceae) increased in abundance in response to treatment with the low group (FIG. 44) compared to the control group. In the high versus control group, only 4 families significantly changed and all increased in response to treatment with the high group (FIG. 45). In the third comparison between high and low groups, 4 families were detected (FIG. 46), half of them increased in the low, while the other half increased in the high group.

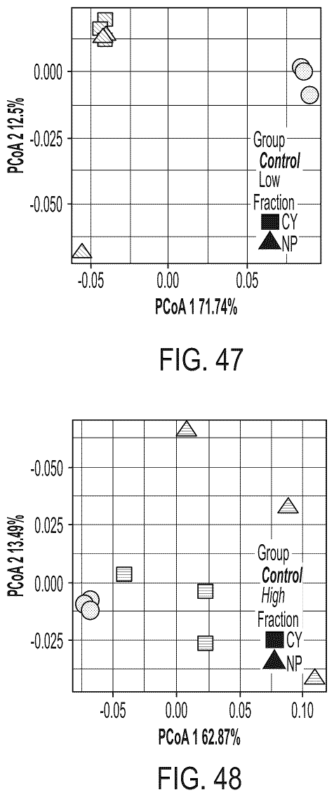

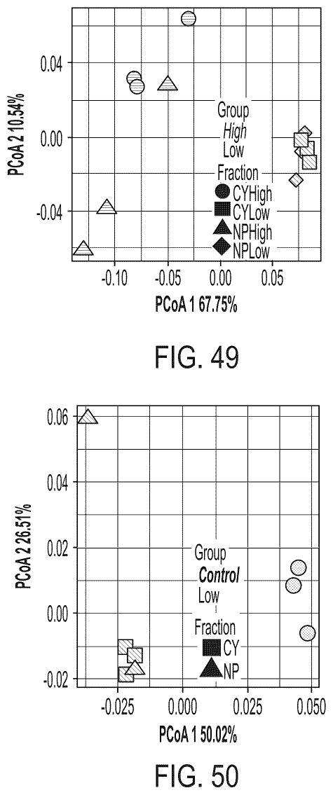

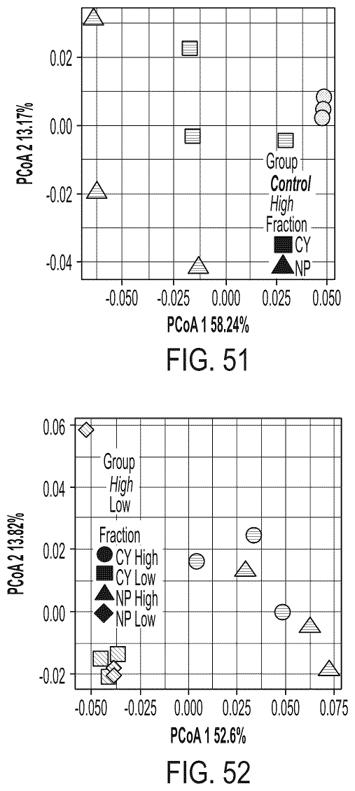

These shifts were detected in the dataset regardless of the pipeline or the read group (forward or reverse) used (FIGS. 47-52).

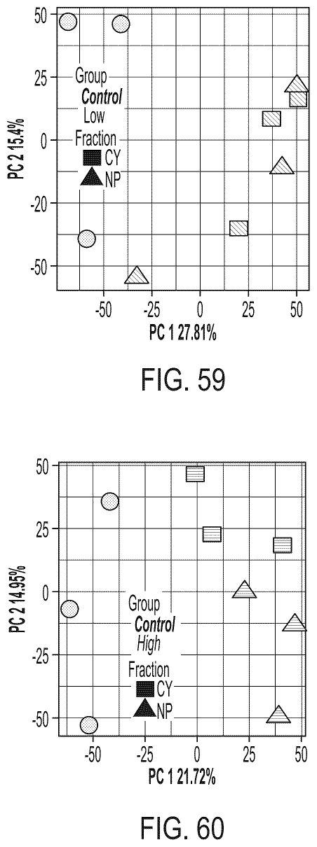

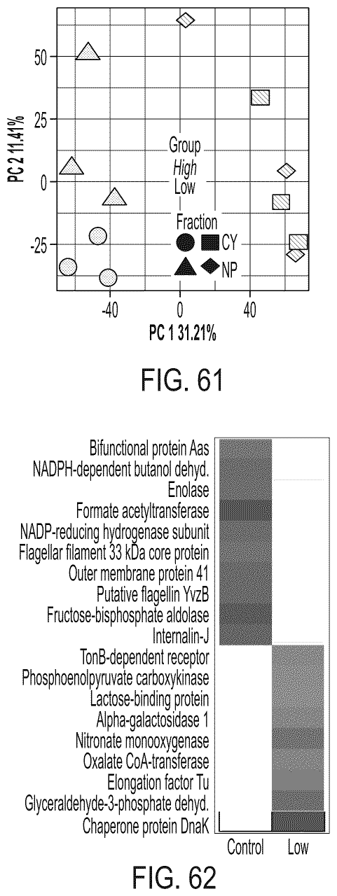

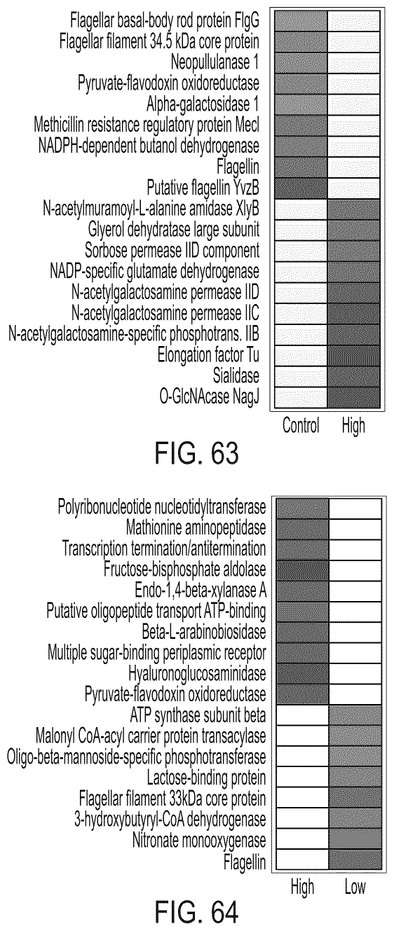

To gain a better understanding of the microbial response to CY and NP exposure, gene expression and pathways were examined. The transcriptomic analysis identified 141 genes differentially expressed between the CY and NP administered in high concentration and control mice (96 genes up-regulated in the high group and 45 up-regulated in the control group). 605 genes differentially expressed between the CY and NP administered in low concentration and control mice (239 genes up-regulated in the low group and 366 up-regulated in the control group) were detected. Regarding the genes differentially expressed between the low and high groups, 10,107 genes differentially expressed with 4,573 genes up-regulated in the low group and 5,534 up-regulated in the high group were found. Principal component analysis (PCA) revealed that microbial transcriptomes of CY and NP administered in low concentration were different from those of control mice (FDR p=0.01) (FIG. 59), CY and NP administered in high concentration were different from those of control mice (FDR p=0.001) (FIG. 60) and CY and NP administered in high concentration were different from those of CY and NP administered in low concentration (FDR p=9.1E-06) (FIG. 61). Interestingly, a number of genes down-regulated in the treated mice are well known bacterial virulence factors, including Enolase, Internalin (FIG. 62), Flagellar basal-body rod protein FlgG and Putative flagellin YvzB (FIG. 63). Enolase is a known immunogenic protein contributing to bacterial virulence in many infectious diseases and Internalins help pathogenic bacteria adhere and invade mammalian cells through E-cadherin. Flagellar genes are among the well-known virulence factors and play important role in bacterial motility, adherence and biofilm formation.

Discussion

Cymopols isolated from the subtropical marine algae Cymopolia barbata were shown to have both antioxidant and anti-inflammatory activities. These cytoprotective properties were validated in various cell culture assays as well as various model organisms including C. elegans and mice. Most of the biological activity was found to be in the digestive tract, namely the small intestine, large intestine, and cecum, associated with increasing transcript levels for cytoprotective genes, Nqo1 and Hmox1. In addition, using RNAseq technology, it was determined that several pathways were affected by cymopol and the NP extract that are associated with oxidative stress, inflammation, and cancer. Most notably, a decrease in pro-inflammatory transcripts, such as that of Tnfα and Cox2, along with concomitant increases in ARE-driven genes, such as Gsta4, was observed. Such compounds could therefore be useful in the prevention and/or treatment of diseases of the digestive system (e.g., Crohn's Disease), which often are associated with oxidative stress and chronic inflammation (e.g., elevated levels of TNFα) [Reimund J M, Ratajczyk J, Sola B, Justum A M, Muller C D (2007) Anti-tumor necrosis factor-alpha (TNF-alpha) strategies in Crohn's disease. Recent Pat Inflamm Allergy Drug Discov (1):21-34]. A particular application could be for prevention of Crohn's disease, in which TNFα mediates chronic inflammation. As with any stress response pathway, the regulation needs to be tightly controlled. Serious considerations for doses and off-target effects would be critical in determining clinical application of cymopols for the prevention of inflammatory mediated disease. However, based on the analyses in vitro and in vivo, cymopols offer great potential as mediators of chronic inflammation and oxidative stress associated with the development of diseases such as colorectal carcinoma. The contribution of the microbiome shift on the additional modulation of host Nrf2 signaling and inflammation is still unclear. It is likely that the microbiome shift leads to a change in the bioactive small molecule content produced by the bacteria, which in turn might not only have secondary effects on the host oxidative stress and anti-inflammatory response but also other disease relevant pathways, which will be explored in future studies. In general, consumption of dietary seaweeds may have functional consequences by modulating host signaling and the microbiome, and the net effect will determine the overall health benefit or potentially adverse effects.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

1. An extract from the algae, Cymopolia sp., isolated by:
a) Exposing said algae to a solvent or solvent combination;
b) Filtering the material/mixture from step a); and
c) Removing the solvent or solvent combination from step b).

2. The extract of claim 1 further comprising:
a) Purifying the material/mixture from step c) within claim 1; and
b) Removing the chromatography mobile phase to provide enriched fractions.

3-6. (canceled)

7. The extract of claim 1, wherein the extract comprises one or more compounds selected from the group consisting of:
a) (E)-2-bromo-5-(3,7-dimethylocta-2,6-dien-1-yl)benzene-1,4-diol (Cymopol, 1);
b) (E)-2-bromo-5-(7-hydroxy-3,7-dimethyloct-2-en-1-yl)benzene-1,4-diol (7-hydroxycymopol, 2);
c) 2,6-dibromo-1,1,4a-trimethyl-2,3,4,4a,9,9a-hexahydro-1H-xanthen-7-ol (Cymobarbatol, 3); and
d) 5-bromo-24(3-bromo-2,2-dimethyl-6-methylenecyclohexyl)methyl)-4-methoxyphenol (Cyclocymopol monomethyl ether, 4).

8. The extract of claim 1, wherein the extract comprises (E)-2-bromo-5-(3,7-dimethylocta-2,6-dien-1-yl)benzene-1,4-diol (Cymopol, 1).

9-63. (canceled)

64. A method of altering the microbiome of the gastrointestinal tract of a subject, the method comprising administering to the subject an effective amount of a composition comprising an extract of claim 1.

65. The method of claim 64, wherein the extract comprises one or more compounds selected from the group consisting of:
e) (E)-2-bromo-5-(3,7-dimethylocta-2,6-dien-1-yl)benzene-1,4-diol (Cymopol, 1);
f) (E)-2-bromo-5-(7-hydroxy-3,7-dimethyloct-2-en-1-yl)benzene-1,4-diol (7-hydroxycymopol, 2);
g) 2,6-dibromo-1,1,4a-trimethyl-2,3,4,4a,9,9a-hexahydro-1H-xanthen-7-ol (Cymobarbatol, 3); and
h) 5-bromo-24(3-bromo-2,2-dimethyl-6-methylenecyclohexyl)methyl)-4-methoxyphenol (Cyclocymopol monomethyl ether, 4).

66. The method of claim 64, wherein the extract comprises (E)-2-bromo-5-(3,7-dimethylocta-2,6-dien-1-yl)benzene-1,4-diol (Cymopol, 1).

67. A method of altering the microbiome of the gastrointestinal tract of a subject, the method comprising administering to the subject an effective amount of a composition comprising an extract of claim 2.

68. The method of claim 67, wherein the extract comprises one or more compounds selected from the group consisting of:
i) (E)-2-bromo-5-(3,7-dimethylocta-2,6-dien-1-yl)benzene-1,4-diol (Cymopol, 1);
j) (E)-2-bromo-5-(7-hydroxy-3,7-dimethyloct-2-en-1-yl)benzene-1,4-diol (7-hydroxycymopol, 2);
k) 2,6-dibromo-1,1,4a-trimethyl-2,3,4,4a,9,9a-hexahydro-1H-xanthen-7-ol (Cymobarbatol, 3); and
l) 5-bromo-2-((3-bromo-2,2-dimethyl-6-methylenecyclohexyl)methyl)-4-methoxyphenol (Cyclocymopol monomethyl ether, 4).

69. The method of claim 67, wherein the extract comprises (E)-2-bromo-5-(3,7-dimethylocta-2,6-dien-1-yl)benzene-1,4-diol (Cymopol, 1).

70. A method of improving the overall health of a subject, the method comprising administering to the subject an effective amount of a composition comprising an extract of claim 1.

71. The method of claim 70, wherein the extract comprises one or more compounds selected from the group consisting of:
m) (E)-2-bromo-5-(3,7-dimethylocta-2,6-dien-1-yl)benzene-1,4-diol (Cymopol, 1);
n) (E)-2-bromo-5-(7-hydroxy-3,7-dimethyloct-2-en-1-yl)benzene-1,4-diol (7-hydroxycymopol, 2);
o) 2,6-dibromo-1,1,4a-trimethyl-2,3,4,4a,9,9a-hexahydro-1H-xanthen-7-ol (Cymobarbatol, 3); and
p) 5-bromo-2-((3-bromo-2,2-dimethyl-6-methylenecyclohexyl)methyl)-4-methoxyphenol (Cyclocymopol monomethyl ether, 4).

72. The method of claim 70, wherein the extract comprises (E)-2-bromo-5-(3,7-dimethylocta-2,6-dien-1-yl)benzene-1,4-diol (Cymopol, 1).

73. A method of improving the overall health of a subject, the method comprising administering to the subject an effective amount of a composition comprising an extract of claim 2.

74. The method of claim 73, wherein the extract comprises one or more compounds selected from the group consisting of:
q) (E)-2-bromo-5-(3,7-dimethylocta-2,6-dien-1-yl)benzene-1,4-diol (Cymopol, 1);
r) (E)-2-bromo-5-(7-hydroxy-3,7-dimethyloct-2-en-1-yl)benzene-1,4-diol (7-hydroxycymopol, 2);
s) 2,6-dibromo-1,1,4a-trimethyl-2,3,4,4a,9,9a-hexahydro-1H-xanthen-7-ol (Cymobarbatol, 3); and
t) 5-bromo-24(3-bromo-2,2-dimethyl-6-methylenecyclohexyl)methyl)-4-methoxyphenol (Cyclocymopol monomethyl ether, 4).

75. The method of claim 73, wherein the extract comprises (E)-2-bromo-5-(3,7-dimethylocta-2,6-dien-1-yl)benzene-1,4-diol (Cymopol, 1).

76. A method of supplementing diet in a subject comprising administering to the subject an effective amount of a composition comprising an extract of claim 1.

77. The method of claim 76, wherein the extract comprises (E)-2-bromo-5-(3,7-dimethylocta-2,6-dien-1-yl)benzene-1,4-diol (Cymopol, 1)

78. A method of supplementing diet in a subject comprising administering to the subject an effective amount of a composition comprising an extract of claim 2.

79. The method of claim 78, wherein the extract comprises (E)-2-bromo-5-(3,7-dimethylocta-2,6-dien-1-yl)benzene-1,4-diol (Cymopol, 1).

80-83. (canceled)
